(19) United States
(12) Patent Application Publication
(10) Pub. No.: US 2021/0190757 A1
(43) Pub. Date: Jun. 24, 2021

(54) NIR-II PHOSPHORESCENT IMAGING PROBE AND METHODS OF IMAGING TISSUE

(71) Applicant: The Board of Trustees of the Leland Stanford Junior University, Stanford, CA (US)

(72) Inventors: Baisong Chang, Wuhan (CN); Zhen Cheng, Stanford, CA (US)

(21) Appl. No.: 17/127,661

(22) Filed: Dec. 18, 2020

Related U.S. Application Data
(60) Provisional application No. 62/951,276, filed on Dec. 20, 2019.

(51) Int. Cl. G01N 33/483; B82Y 30/00; G01N 21/64

(52) U.S. Cl. CPC G01N 33/4833; G01N 2800/7028; G01N 21/6486; B82Y 30/00

(57) ABSTRACT

The disclosure provides NIR-II phosphorescent imaging probe and methods of using the NIR-II phosphorescent imaging probes for imaging tissues, such as cancerous tissues. NIR-II phosphorescent imaging probes of the present disclosure include CuInX₂ nanotubes, where X is a chalcogen selected from S, Se, and Te, such as CuInSe₂ nanotube.

### CROSS-REFERENCE TO RELATED APPLICATIONS

This application claims the benefit of and priority to co-pending U.S. Provisional Patent Application No. 62/951,276, filed on Dec. 20, 2019, entitled “NIR-II PHOSPHORESCENT IMAGING PROBE AND METHODS OF IMAGING TISSUE,” the contents of which is incorporated by reference herein in its entirety.

### STATEMENT REGARDING FEDERALLY SPONSORED RESEARCH OR DEVELOPMENT

This invention was made with Government support under grant DESC0008397 awarded by the US Department of Energy. The Government has certain rights in the invention.

### BACKGROUND

Optical imaging probes including semiconducting quantum dots and rare-earth nanocrystals have been extensively studied for early diagnosis and prognosis evaluation of tumors and other human diseases. Specifically, fluorescence bioimaging in the second near-infrared window (NIR-II, 1000-1700 nm) is an actively studied technique with much promise. However, nearly all such probes suffer from the drawback of being “always on,” continuously producing unspecific luminescent signal regardless of whether they reach the target pathological locations. Thus, in vivo tumor imaging by common optical probes always suffers from poor detection precision due to unsatisfactory tumor-to-background signal ratios. Also, it is a classic probe-application dilemma that the two typical requirements, high tumor imaging selectivity and low noise interference from autofluorescence and normal organs, tend to be mutually exclusive.

Tumor accumulation of nano-sized probes based on the enhanced permeability and retention (EPR) effect, a concept that theoretically enables clearer and deeper imaging into tumors, is time-dependent and thus relies on probes with a longer circulation half-life. Unfortunately, prolonged circulation time will inevitably produce background interference noises from “always on” probes, especially in mononuclear phagocyte systems (MPS), such as liver and spleen organs. Given the presumed critical role of EPR effect, it remains a topic of debate in nanomedicine. Most reports support EPR-induced preferential tumor accumulation of nanomaterials, whereas others claim the EPR effect does not work in most solid tumors. From a pathological point of view, intra- and intertumoral heterogeneities are a principal etiology that influences EPR effect, varying over time in tumor development and possibly being transient. It is thereby desirable to develop a new imaging strategy for specifically viewing tumor tissue without background noise while offering constructive (e.g., quantitative) guidelines on the clinical translation of EPR-associated nanomedicines.

### SUMMARY

In various aspects described herein, imaging probes, pharmaceutical imaging compositions, imaging systems, methods of imaging tissues, and methods of making imaging probes are provided.

In some aspects described herein, the present disclosure provides imaging probes, where an imaging probe includes a CuInX₂ nanotube, where X is a chalcogen selected from S, Se, and Te, the CuInX₂ nanotube has an outer diameter and an inner diameter defining a hollow center, where the CuInX₂ nanotube emits a weak florescence at a pH of about 7.0 or higher and is configured to emit detectable NIR-II phosphorescence upon aggregation with a plurality of other CuInSe₂ nanotubes at a pH of about 6.8 or lower. According to some aspects of the disclosure the imaging probes are CuInSe₂ nanotubes. Aspects of the present disclosure also include pharmaceutically acceptable imaging compositions including a plurality of imaging probes of the disclosure and a pharmaceutically acceptable carrier.

Methods of generating an image of a tissue in an animal or human subject are also provided in the present disclosure. Aspects of such methods include administering to an animal or human subject a pharmaceutically acceptable composition comprising a plurality of CuInX₂ nanotubes of the present disclosure and obtaining an image of the location of nanoaggregates of the CuInX₂ nanotubes in a tissue of the animal or human subject by detecting and imaging the phosphorescence. In the methods using the CuInX₂ nanotubes, X is a chalcogen selected from S, Se, and Te, and the CuInX₂ nanotubes emit weak fluorescence at an environmental pH of about 7.0 or higher, and where the CuInX₂ nanotubes form nanoaggregates and emit NIR-II phosphorescence in a second near-infrared range of about 1000-1700 nm at an environmental pH of about 6.8 or lower. According to aspects of the disclosure, the X for the CuInX₂ nanotubes is Selenium, and the CuInX₂ nanotube is a CuInSe₂ nanotube.

The present disclosure further provides systems for generating an image of a tissue in an animal or human subject, where, in embodiments, the system includes a pharmaceutically acceptable imaging composition including a plurality of imaging probes as described above in a pharmaceutically acceptable carrier and an imaging system configured to detect phosphorescence in a second near infrared range of about 1000-1700 nm.

According to other aspects of the present disclosure, methods of making CuInSe₂ nanotubes of the present disclosure are provided. In embodiments, the method includes the steps of: synthesizing Cu₂₋ₓSe solid nanorods by water-evaporation-induced self-assembly, and reduction of the Cu₂₋ₓSe nanorods with NaBH₄ to form hollow CuInSe₂ nanotubes.

Other systems, methods, features, and advantages of the coating compositions, coated articles, and methods of making thereof will be apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1.1A-1.1E illustrate various characterizations of CuInSe₂ (“CISe”) nanotubes. FIG. 1.1A is a schematic illustrating the working principle of CISe nanotubes as a NIR-II phosphorescent probe. CISe nanotubes can efficiently switch radiative mode from extremely weak fluorescence to strong NIR-II phosphorescence upon receiving subtle triggers in physiologically relevant pH range, showing significant increases in emission lifetime and intensity, respectively. FIG. 1.1B is a TEM image, and FIGS. 1.1C and 1.1D are HR-TEM images of CISe nanotubes. The dashed circle in FIG. 1.1C represented the end of CISe nanotubes. FIG. 1.1E illustrates a series of HAADF-STEM images. The energy-dispersive X-ray spectroscopy elemental maps in e displayed the distribution of Cu (red), In (green) and Se (yellow), respectively (colors not shown).

FIGS. 1.2A-1.2N generally illustrate the photophysical properties of CISe nanotubes. FIG. 1.2A is a schematic illustration of intensity-based NIR-II imaging system. FIG. 1.2B is a series of NIR-II intensity images (808 nm excitation, 0.14 W cm⁻² power density, 5 ms exposure time and 1000 nm long-pass optical filter) of CISe nanotubes in different PBS and solid powder. The stick figure of “bulb” in FIB. 1.2B was in advance drawn using solid powder of CISe nanotubes as a “ink”. FIG. 1.2C is an emission spectra of CISe nanotubes under 808 nm excitation in different PBS. FIG. 1.2D is a plot of PL intensity enhancements of CISe nanotubes and PEG-CIS nanorods versus a broad range of pH values, respectively. I_{(pH 7.4)} was set as the starting PL intensity of probe and I was the PL intensity at other decreased pH values. FIG. 1.2E illustrates cartoons (top) and TEM images (bottom) of self-assembly states of CISe nanotubes in different PBS. The samples were placed without appreciable disturbance for about 2 h before TEM measurements. FIG. 1.2F is a schematic illustration of H-bonding-driven reversible self-limited assembly of CISe nanotubes responding to pH. The right column showed the mechanism of interactions underlying H-bonding-driven self-limited assembly. Three green circles denoted deprotonated sites in the pH range where CISe nanotubes can be highly stabilized, while the yellow rectangle denoted interparticle H-bonding interactions in this range. FIG. 1.2G is a schematic illustration of time-resolved NIR-II imaging system. FIG. 1.2H is a series of NIR-II lifetime images (808 nm excitation, 0.14 W cm⁻² power density and 50 μs delay time) of CISe nanotubes in different PBS and solid powder, demonstrating that the long-lived phosphorescence can remarkably improve imaging sensitivity. FIGS. 1.21 and 1.2J are PL decay profiles detected at 1010 nm (1.21) and 1130 nm (1.2J) of CISe nanotubes in different PBS. The red solid lines were the fitted curves by different exponential decay functions (see FIGS. 2.14, 2.15 from Ex. 2). FIG. 1.2K is a bar graph of lifetimes calculated from PL decay profiles in FIGS. 1.21 and 1.2J. N/A represented “not applicable”, corresponding to no phosphorescence signal. FIG. 1.2L is a plot of lifetime enhancements of CISe nanotubes and PEG-CIS nanorods versus a broad range of pH values, respectively. Of note, τ_{(pH 7.4)} corresponded to the lifetime of 1010 nm peak of CISe nanotubes in pH 7.4 and T was derived from 1130 nm peak of CISe nanotubes in other decreased pH values. FIG. 1.2M illustrates cycling experiments of pH-dependent PL switch. Stemming from pH-induced breakage and reconstruction of dynamic H-bonding networks, both PL intensities (top) and lifetimes (bottom) of CISe nanotubes rapidly returned to their original states and benefited excellent reversibility. FIG. 1.2N are graphs illustrating photostability of CISe nanotubes in fetal bovine serum (FBS) and PBS with different salt concentrations (0.1 and 1.0 M) under pH 6.0 and a continuous 808 nm illumination.

FIGS. 1.3A-1.31 illustrate a possible mechanism of the generation of NIR-II phosphorescence. FIGS. 1.3A and 1.3D illustrate 3D transient absorption (TA) spectra monitored as a function of pump-probe delay time and probe wavelength for CISe nanotubes in pH 7.4 (1.3A) and 6.0 (1.3D), respectively. The pumped wavelength was 400 nm with a pulse energy of 20 ρW. FIGS. 1.3B and 1.3E illustrate evolution-associated TA spectra that followed from a global analysis of data for CISe nanotubes in pH 7.4 (1.3B) and 6.0 (1.3E), respectively. FIGS. 1.3C and 1.3F illustrate dynamics taken from GSB and ESA signals with kinetic traces (hollow circles) and fits (solid lines) for CISe nanotubes in pH 7.4 (1.3C) and 6.0 (1.3F), respectively. A series of evolution-associated time constants in FIG. 1.3F, which represented the characteristic dynamics of different electronic states for CISe nanotubes in pH 6.0, matched well with a sequential decay model. FIG. 1.3G is a HR-TEM image of cracked CISe nanotubes upon grinding. The arrows indicated In-rich domain. Inset: cartoon showing many In-rich domains distributed in CISe nanotubes. HR-TEM image appeared in blue/orange pseudo-color to identify In-rich domains clearly. FIG. 1.3H illustrates low-temperature PL spectra of CISe nanotubes in solid states from 4 to 300 K. Inset: thermal quenching properties of PL and the fitted parameter of activation energy (ΔE) for CISe nanotubes from 80 to 300 K. FIG. 1.31 is a schematic illustration of processes after photoexcitation that accounted for the generation of NIR-II phosphorescence, where the S₀, S₁ and T₁ represented ground state, excited singlet state and excited triplet state, respectively.

FIGS. 1.4A-1.4D illustrate in vivo biosafety evaluation of CISe nanotubes. FIG. 1.4A is a schematic illustration of a blood collection scheme for mice intravenously treated with CISe nanotubes (100 mg kg⁻¹). At selected post-injection times, the mice were anaesthetized and eyeballs were removed, followed by the collection of blood samples for blood chemistry tests. FIGS. 1.4B and 1.4C illustrate testing of a series of Hepatic function markers (aspartate aminotransferase, AST; alkaline phosphatase, ALP; alanine aminotransferase, ALT; alanine albumin, ALB; albumin/globulin, A/G; cholinesterase, CHE; monoamine oxidase, MAO; and total bilirubin, TBil), renal function markers (total protein, TP; urea; and glutamyltransferase, GGT) and heart function marker (creatinine, CRE). These were tested and found to be normal compared with the control group after 7 and 30 days, respectively, indicating no noticeable hepatic and renal dysfunctions induced by CISe nanotubes. Note that the indicator of TBil not only indicates the function of liver, but also reflects whether the hemolysis occurs in the body. FIG. 1.4D is a series of H&E-stained images of tissues (heart, liver, spleen, lung and kidney) of the mice harvested from the control and 30 days after intravenous injection of CISe nanotubes (100 mg kg⁻¹). The mice treated with PBS were used as the blank control. n=5. The scale bar corresponded to 500 μm.

FIGS. 1.5A-1.5L illustrate aspects of ultrahigh tumor-specific imaging by NIR-II phosphorescence. FIGS. 1.5A and 1.5B illustrated the working principle of CISe nanotubes for intensity-based NIR-II imaging (1.5A) and time-resolved NIR-II imaging (1.5B), respectively. (N−1)Δt in FIG. 1.5B was the delay time before the charge-coupled device (CCD) capturing the next image. FIGS. 1.5C and 1.5D illustrate time-dependent NIR-II intensity images (left) and lifetime images (right) of the nude mice bearing 143B osteosarcoma cancer xenografts received an intravenous injection of CISe nanotubes (1.5C) and PEG-CIS nanorods (1.5D), respectively (dosage: 20 mg kg⁻¹). The delay time was 50 μs for lifetime imaging. The circle and square corresponded to tumor site and normal tissue, respectively, where their PL intensities were selected to calculate T/NT ratios in FIG. 1.5K. FIG. 1.5E shows representative NIR-II intensity images (left) and lifetime images (right) of the tumor-bearing nude mice treated with different nanoprobes in the presence of glycolysis inhibitors. Notably, 2-DG or CHC (dosage: 250 mg kg⁻¹) was injected 12 h before probe administration with a dosage of 20 mg kg⁻¹. FIG. 1.5F is a simplified version of metabolic pathways resulting in the conversion of glucose to metabolic products such as lactate in cancer cells. 2-DG and CHC are the two typical metabolic inhibitors. 2-DG inhibits glucose uptake by cell surface glucose transporter and also reduces phosphorylation via hexokinases; CHC is a monocarboxylate transporter inhibitor that avoids lactic acid secretion from tumor cells. FIGS. 1.5G and 1.5H are post-necropsy in situ imaging of tumor mice after resecting tissues and ex vivo imaging of resected organs including tumors after 24 h post-injection of CISe nanotubes (1.5G) and PEG-CIS nanorods (1.5H), respectively. FIG. 1.51 illustrates lifetime distributions of CISe nanotubes-treated tumor-bearing nude mice in FIG. 1.5C (lifetime images) against time from 2 to 24 h post-injection. FIG. 1.5J is a cross-sectional PL intensity profile along the dashed line in high-magnification tumor vascular imaging (inset) of tumor-bearing mouse after 24 h post-injection of CISe nanotubes. Gaussian fit profile was shown in the graph line. FIG. 1.5K is a graph of T/NT ratios by intensity imaging against time over a course of 24 h post-injection of probes in FIGS. 1.5C and 1.5D, respectively. Note that T/NT ratios from CISe nanotubes surpassing the Rose criterion lasted at least 19 h, affording a long-time window to identify tumor-involved details such as tumor vasculature variations. FIG. 1.5L is a graph of quantitative PL intensity ratios of tumor to normal tissues in FIGS. 1.5G and 1.5H, respectively. In each group, experiment performed with 5 independent mice with similar results. All the results were presented as the mean±s.d. ***P<0.001, ** P<0.01.

FIGS. 1.6A-1.6K illustrate superior penetration depth and resolution of NIR-II phosphorescent imaging. FIG. 1.6A is a series of digital photographs and phantom study comparing penetration depths of CISe nanotubes and PEG-CIS nanorods in PBS with pH 5.5 at depths of 0, 3 and 5 mm (1% Intralipid) under 808 nm excitation (0.14 W cm⁻² power density, 5 ms exposure time and 1000 nm long-pass optical filter). FIGS. 1.6B-1.6D illustrate the cross-sectional intensity profiles of CISe nanotubes (higher peaks) and PEG-CIS nanorods (lower peaks) along the dashed arrows in NIR-II images (1.6A) at depths of 0 (1.6B), 3 (1.6C) and 5 mm (1.6D), respectively. Notably, taking 2 mg mL⁻¹ of nanoprobes as an example, quantitative analysis of imaging quality was performed by fitting the recorded intensity profiles with the Gaussian function (black). FIGS. 1.6E-1.6G illustrate graphs of PL signal intensities (1.6E), FWHM (1.6F) and signal-to-noise ratios (1.6G) of cross-sectional profiles in capillary images as a function of depths (0-8 mm). At penetration depth of 8 mm, FWHM values cannot be calculated owing to the extremely weak PL signal intensities. The signal-to-noise ratio was estimated by dividing the average signal intensities of capillaries (5 pixels width) by average background (noise) signal intensities (5 pixels width, 20 pixels apart from the capillary signals). FIG. 1.6H is a schematic illustration and digital photographs of tumor-bearing nude mice injected with probes intratumorally, showing different injection sites: close to skin surface (˜1 mm depth), the upper and lower parts of tumor tissue (˜3 and ˜5 mm depths including skin thickness, respectively). FIG. 1.61 are the corresponding NIR-II images of tumor-bearing nude mice injected with CISe nanotubes and PEG-CIS nanorods as a function of depth (0-5 mm), respectively. FIG. 1.6J illustrates the cross-sectional intensity profiles along the dashed lines shown in NIR-II images (1.61). FIG. 1.6K is a graph illustrating the PL signal intensities (left) and signal-to-noise ratios (right) of cross-sectional profiles as a function of depth (0-5 mm). The bars represented mean±s.d. derived from n=5 biologically independent mice.

FIGS. 2.1A-C illustrate synthesis and characterization of Cu_{1.8}Se nanorods as a template. FIGS. 2.1A and 2.1B are a SEM image (2.1A) and TEM image (2.1B) of Cu_{1.8}Se nanorods. Note: for SEM characterization, in order to precisely achieve structural information, the samples were observed without sputter coating pretreatment. FIG. 2.1C illustrates the corresponding EDS spectrum in FIG. 2.1B. The EDS measurement was performed on regions containing a large number of samples to obtain sufficient signals and was repeated for three different areas of the TEM grid.

FIGS. 2.2A-2.2C illustrate molecular characterizations of CISe nanotubes, showing High-resolution XPS spectra (2.2A), FT-IR spectra (2.2B), and XRD pattern (2.2C).

FIGS. 2.3A-2.3F illustrate optimization of Cu⁺-to-In³⁺ cation exchange and proposed mechanism of hollowing CISe nanotubes. FIGS. 2.3A-2.3C represent typical HR-TEM images involved in the formation of CISe nanotubes at a series of reaction times from 0.5 h (2.3A), 1 h (2.3B) to 1.5 h (2.3C). Insets: the borderlines between CuInSe₂ and In₂Se₃ nanocrystallites as indicated via the dotted white line. The sporadic regions marked with the solid, straight arrows in FIG. 2.3A suggested that Cu⁺-to-In³⁺ cation exchange reactions initiated on the edges of Cu_{1.8}Se nanorods. FIG. 2.3D is a schematic illustration of elementary kinetic steps in conversion of solid Cu_{1.8}Se nanorods into hollow CISe nanotubes following Cu⁺-to-In³⁺ cation exchange process, namely inward diffusion of In³⁺ cation and outward diffusion of Cu⁺ cation. FIG. 2.3E is a TEM image of intermediates collected from the extension of cation exchange reactions at 3 h. FIG. 2.3F illustrates a typical corresponding EDS spectrum in FIG. 2.3C. The EDS measurement was performed on regions containing a large number of samples in order to achieve sufficient signals and was repeated for three different areas of the TEM grid.

FIGS. 2.4A-2.4C illustrate preparation of PEG-CIS nanorods as a control NIR-II fluorescent probe. FIGS. 2.4A and 2.4B are TEM and HR-TEM images of PEG-CIS nanorods. FIG. 2.4C is a drawing showing the morphology of PEG-CIS nanorods.

FIGS. 2.5A-2.5D illustrate photophysical properties of PEG-CIS nanorods as a control probe. FIGS. 2.5A and 2.5B are NIR-II intensity images (2.5A) (808 nm excitation, 0.14 W of cm⁻² power density, 5 ms exposure time, 1000 nm long-pass optical filter) and NIR-II lifetime images (2.5B) (808 nm excitation, 0.14 W cm⁻² power density, 50 μs delay time) of PEG-CIS nanorods in different pH values. FIGS. 2.5C and 2.5D are emission spectra (2.5C) and PL decay curves (2.5D) of PEG-CIS nanorods in pH 8.0, 7.4 and 5.5, respectively.

FIG. 2.6 is a PL spectra of CISe nanotubes in PBS with pH 7.4, showing an extremely weak emission intensity.

FIGS. 2.7A and 2.7B illustrate evaluation of the colloidal stability of CISe nanotubes. FIGS. 2.7A illustrates an acid-base titration curve of CISe nanotubes, and FIG. 2.7B is a graph of Zeta potentials of CISe nanotubes in a broad pH range.

FIGS. 2.8A-2.8C illustrate evaluation of self-assembly of CISe nanotubes in PBS with different pH values. FIGS. 2.8A-2.8C are AFM images of CISe nanotubes in pH 7.4 (2.8A), 6.0 (2.8B) and 5.0 (2.8C), respectively. At pH 6.0, CISe nanotubes self-assembled into stable clusters. By lowering pH to 5.0, despite the self-limited assembly of CISe nanotubes quickly deteriorated to some extent, these clusters can still maintain a compact arrangement of assembly structures over a longer time.

FIG. 2.9 is a schematic illustration of the creation of supraparticles or uncontrolled aggregations using the nanoparticles as elementary functional and structural units. The basic strategy underlying the generation of colloidal supraparticles was to use bottom-up method for assembling individually characterized nanoscale building constructs, which together executed a controllable function.

FIGS. 2.10A and 2.10B illustrate a comparison of PL of CISe nanotubes in different states. FIG. 2.10A illustrates NIR-II images (5 ms exposure time, 1000 nm long-pass optical filter, 808 nm excitation, 0.14 W cm⁻² power density) of CISe nanotubes in pH 5.5 PBS and in solid powders, respectively. FIGS. 2.10B is a corresponding emission spectra of CISe nanotubes. It was worth noticing that the PL intensity of CISe nanotubes in solid powders was much stronger than in PBS even with a pH of 5.5.

FIGS. 2.11A-2.11F illustrate possible multiple H-bonding interactions between GSH ligands from shells of two approaching CISe nanotubes. The formed H-bonds were marked using the green-dashed lines. FIGS. 2.11A-2.11B, Single H-bond; FIGS. 2.11C-2.11E, Double H-bonds; and FIG. 2.11F, Triple H-bonds.

FIGS. 2.12A-2.12F illustrate quantum yield determination of CISe nanotubes. FIGS. 2.12A-2.12D are UV-Vis spectra of IR-26 (2.12A) and CISe nanotubes (2.12B), PL spectra of IR-26 (2.12C) and CISe nanotubes (2.12D), respectively. Absorbance values at 808 nm and area under curve in PL spectra were calculated and listed in inset table. FIGS. 2.12E and 2.12F are graphs of Integrated emission intensities of IR-26 (2.12E) and CISe nanotubes (2.12F) as a function of UV-Vis absorbance values at 808 nm. The data were fitted into a linear function with a slope.

FIGS. 2.13A and 2.13B illustrate quantum yield determination of CISe nanotubes in pH 7.0 and 6.5. As clearly evidenced by NIR-II emission spectra, CISe nanotubes in pH 7.0 (2.12A) and 6.5 (2.13B) showed two main emission peaks, i.e. 1010 nm fluorescence and 1130 nm phosphorescence. The mixed PL spectra should be carefully separated before measuring the QY associated with fluorescence and phosphorescence, respectively. Guided by this point, the area values under fluorescent and phosphorescent curves of CISe nanotubes will be separately plotted against the absorbance at 808 nm and fitted into a linear function, as slopes were indicated.

FIGS. 2.14A-2.14F illustrate summarization of the emission decay profiles of CISe nanotubes in a series of pH values monitored at about 1010 nm. 2.14A, pH 7.4; 2.14B, pH 7.2; 2.14C, pH 7.0; 2.14D, pH 6.8; 2.14E, pH 5.5; and 2.14F, pH 5.5. The tables in insets showed the lifetime components.

FIGS. 2.15A-2.15E illustrate summarization of the emission decay profiles of CISe nanotubes in a series of pH values monitored at about 1130 nm. 2.15A, pH 7.2; 2.15B, pH 7.0; 2.15C, pH 6.8; 2.15D, pH 6.5; and 2.15E, pH 5.5. The tables in the inset showed the lifetime components.

FIGS. 2.16A-2.16F illustrate normalized PL decay curves multiplied by the decay time (Higashi-Kastner plots) for the CISe nanotubes monitored at ˜1010 nm. FIGS. 2.16A-2.16F represent the CISe nanotubes in various PBS: 7.4 (2.16A), 7.2 (2.16B), 7.0 (2.16C), 6.8 (2.16D), 6.5 (2.16E) and 5.5 (2.16F). Of note, peak positions represented the most dominant decay time.

FIGS. 2.17A-2.17E illustrate normalized PL decay curves multiplied by the decay time (Higashi-Kastner plots) for the CISe nanotubes monitored at ˜1130 nm, where FIGS. 2.17A-2.17F represent CISe nanotubes in various PBS: 7.2 (2.17A), 7.0 (2.17B), 6.8 (2.17C), 6.5 (2.17D) and 5.5 (2.17E). Of note, peak positions represented the most dominant decay time.

FIGS. 2.18A and 2.18B illustrate characterization of absorption properties of CISe nanotubes. UV-Vis spectra (2.18A) and excitation spectra (2.18B) of CISe nanotubes dispersed in PBS with three representative pH values.

FIGS. 2.19A-2.19C illustrate low-temperature PL spectra of CISe nanotubes. FIG. 2.19A is a temperature-dependent steady-state PL spectra of CISe nanotubes in solid states from 4 to 300 K. FIG. 2.19B is a temperature-dependent intensity of maximum emission signals in 2.19A. FIG. 2.19C is a graph of thermal quenching properties of PL. The fitted parameter of activation energy (4E) for CISe nanotubes from 80 to 300 K.

FIG. 2.20 illustrates low-temperature PL spectra of PEG-CIS nanorods as a control. Temperature-dependent steady-state PL spectra of PEG-CIS nanorods in solid states from 4 to 300 K.

FIG. 2.21 illustrates evaluation of photostability of CISe nanotubes. The brightness of CISe nanotubes could maintain constant in warm (37° C.) FBS and PBS at different pH values under a continuous irradiation of an 808 nm laser (140 mW cm⁻²) for 500 min.

FIG. 2.22 illustrates PL decay curves of CISe nanotubes in solid powders. The PL peak at 1140 nm decayed triple-exponentially with a substantially longer lifetime of 381 μs and high quantum yield of 39.8% (see Table 2).

FIGS. 2.23A and 2.23B illustrated concentration-dependent PL spectra of CISe nanotubes. The PL spectra (2.23A) and representative NIR-II images (2.23B) of CISe nanotubes dispersed in pH 6.8 with a series of concentrations. The NIR-II images in FIG. 2.23B were captured by an 808 nm irradiation (5 ms exposure time, 0.14 W cm⁻² power density) with 950 nm (top) and 1100 nm (bottom) long-pass filters, respectively.

FIG. 2.24A-2.24C illustrate concentration-dependent PL properties of PEG-CIS nanorods. FIG. 2.24A is a series of NIR-II images of PEG-CIS nanorods with different concentrations that were captured using an 808 nm illumination (5 ms exposure time, power density of 140 mW cm⁻²) under 900 nm (left), 1000 nm (middle) and 1100 nm (right) long-pass filters, respectively. FIG. 2.24B is the corresponding PL emission spectra of PEG-CIS nanorods with different concentrations. It can be found that the positions of emission peak remained constant at 1035 nm when the concentrations of PEG-CIS nanorods changed from 0.1 to 5.0 mg mL⁻¹. However, as was evident in the graph of FIG. 2.24C, the emission maxima showed a linear correlation with the concentrations of PEG-CIS nanorods.

FIGS. 2.25A-2.25C illustrate in vitro biosafety evaluation of CISe nanotubes. The relative cell viabilities of normal 3T3 cells (2.25A), tumor MCF-7 cells (2.25B) and tumor 143B cells (2.25C) treated by CISe nanotubes with a series of concentrations after 24 h (left) and 48 h (right) incubations.

FIG. 2.26 illustrates in vitro biosafety evaluation of CISe nanotubes. Body weight changes of healthy mice with and without intravenous injections of CISe nanotubes (n=5 mice in each group) over a period time of 30 days. The dosage of CISe nanotubes was 100 mg kg⁻¹.

FIGS. 2. 27A and 2.27B illustrate through-skin, in vivo real-time tumor vascular imaging. The images in FIG. 2. 27A show high-magnification NIR-II intensity imaging of subcutaneous 143B osteosarcoma tumors on a mouse at different time points after the intravenous injection of CISe nanotubes with a dosage of 20 mg kg⁻¹. FIG. 2.27B is a diagram illustrating tumor vascular networks.

FIGS. 2.28A-2.28F illustrate quantitative characterizations of intensity-based NIR-II imaging in FIGS. 1.5C and 1.5D from Example 1. The cross-sectional PL intensity profiles (lower panel) along dashed lines in intensity-based NIR-II images (upper panel, FIGS. 1.5C and 1.5D) at different post-injection times of probes. CISe nanotubes: 2 h (2.28A), 12 h (2.28B) and 24 h (2.28C); PEG-CIS nanorods: 2 h (2.28D), 12 h (2.28E) and 24 h (2.28F). Gaussian fit profile was shown in black line.

FIGS. 2.29A-2.29D illustrate tumor suppressors exertion of direct effects on metabolism. FIGS. 2.29A and 2.29B illustrate the effects of 2-DG or CHC on the secretions of lactic acid in tumor 143B cells (2.29A) and tumor MCF-7 cells (2.29B), respectively. FIGS. 2.29C and 2.29D show acidification of culture mediums of tumor 143B cells (2.29C) and tumor MCF-7 cells (2.29D) in the presence of 2-DG or CHC after 12 h incubation. The mean±s.d. from 5 independent replicates was shown. **P<0.01, ***P<0.001, compared with the control group. Data were normalized on total protein content.

FIGS. 2.30A-2.30C are supplemental images to the NIR-II imaging shown in FIG. 1.5C of Example 1. Images under white light of the nude mice bearing 143B osteosarcoma cancer xenografts in the lateral (2.30A), prone (2.30B) and supine (2.30C) positions, respectively.

FIGS. 2.31A-2.31B are supplemental images to the NIR-II imaging shown in FIGS. 1.5G and 1.5H of Example 1. Microscopic imaging of resected tumor and main organs under a white light after 24 h post-injection of CISe nanotubes (2.31A) and PEG-CIS nanorods (2.31B), respectively. Scale bar: 1 cm.

FIGS. 2.32A-2.32C illustrate use of CISe nanotubes as a NIR-II phosphorescent nanoprobe for the broad tumor-specific imaging. FIGS. 2.32A and 2.32B are time-dependent NIR-II intensity images of the mice bearing MCF-7 cancer xenografts received intravenous injection of PEG-CIS nanorods (2.32A) and CISe nanotubes (2.32B) with dose of 20 mg kg⁻¹, respectively. 2.32C is a series of representative NIR-II intensity images of tumor-bearing nude mice treated with CISe nanotubes in the presence of glycolysis inhibitors. Notably 2-DG or CHC (dose: 250 mg kg⁻¹) was injected 12 h before administration of CISe nanotubes with dose of 20 mg kg⁻¹.

FIGS. 2.33A-2.33C are supplemental images to the NIR-II imaging shown in FIGS. 1.6H and 1.61 of Example 1. FIG. 2.33A is a set of digital photographs of the dissected tumor-bearing nude mice treated with probes at various sites. From left to right: probes covered with ˜2.3, 3.5 and 4.1 mm thickness of skin and tumor, respectively. FIGS. 2.33B and 2.33C illustrate NIR-II imaging of tumor-bearing nude mice injected with CISe nanotubes (2.33B) and PEG-CIS nanorods (2.33C) at various sites denoting three typical imaging depths.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, nanochemistry, organic chemistry, biochemistry, molecular biology, pharmacology, toxicology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20-25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions

In describing and claiming the disclosed subject matter, the following terminology will be used in accordance with the definitions set forth below.

As used herein, “about,” “approximately,” and the like, when used in connection with a numerical variable, generally refers to the value of the variable and to all values of the variable that are within the experimental error (e.g., within the 95% confidence interval for the mean) or within +1-10% of the indicated value, whichever is greater.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

The term “pharmaceutically acceptable” describes a material that is not biologically or otherwise undesirable, i.e., without causing an unacceptable level of undesirable biological effects or interacting in a deleterious manner.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “nanoparticle” as used herein includes a nanoscale deposit of homogenous or heterogeneous material. Nanoparticles may be regular or irregular in shape and may be formed from a plurality of co-deposited particles that form a composite nanoscale particle. Nanoparticles may be generally spherical in shape or have a composite shape formed from a plurality of co-deposited generally spherical particles. Exemplary shapes for the nanoparticles include, but are not limited to, spherical, rod, elliptical, cylindrical, disc, and the like. In some embodiments, the nanoparticles have a substantially spherical shape. The term “nanoparticle” generally refers to a particle having a diameter of between about 1 and about 1000 nm. Similarly, by the term “nanoparticles” is meant a plurality of particles having an average diameter of between about 1 and about 1000 nm.

It will be understood by one of ordinary skill in the art that when referring to a population of nanoparticles as being of a particular “size”, what is meant is that the population is made up of a distribution of sizes around the stated “size”. Unless otherwise stated, the “size” used to describe a particular population of nanoparticles will be the mode of the size distribution (i.e., the peak size). By reference to the “size” of a nanoparticle is meant the length of the largest straight dimension of the nanoparticle. For example, the size of a perfectly spherical nanoparticle is its diameter.

The term “detectable” refers to the ability to detect a signal over the background signal. The detectable signal is defined as an amount sufficient to yield an acceptable image using equipment that is available for pre-clinical use. A detectable signal maybe generated by one or more administrations of the probes of the present disclosure. The amount administered can vary according to factors such as the degree of susceptibility of the individual, the age, sex, and weight of the individual, idiosyncratic responses of the individual, the dosimetry, and the like. The amount administered can also vary according to instrument and digital processing related factors. In embodiments the signal is a phorphorescent signal and is detected by an imaginag systems configured to detect phosphorescence in a second near infared range (e.g., about 1000-1700 nm).

The term “in vivo imaging” as used herein refers to methods or processes in which the structural, functional, or physiological state of a living being is examinable without the need for a life-ending sacrifice.

The term “non-invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The term “detectable imaging moiety,” “imaging probe,” “detectable label” or “label” as used herein refers to an atom, or radioactive atom detectable by systems and methods such as, but not limited to, optical detection, γ-radiation detection, positron emission transmission, and the like. Some inorganic or organic molecules may be detected by an optical method, for example by fluorescence detection, light absorbance and the like. It should be noted that reference to detecting a signal from a probe also includes detecting a signal from a plurality of probes. In some embodiments, a signal may only be detected that is produced by a plurality of probes (e.g., nanoaggregates). Additional details regarding detecting signals (e.g., infared signals) are described below.

The “imaging probe” may be detected either externally to a subject human or non-human animal body or via use of detectors designed for use in vivo, such as intravascular radiation or optical detectors such as endoscopes, or radiation detectors designed for intra-operative use. The imaging moiety is preferably chosen from, the NIR-II phosphorescent emitting nanomaterials of the present disclosure reporter suitable for in vivo optical imaging. It is contemplated, however, that other detectable labels may be incorporated into the probes of the disclosure including, but not limited to, a radioactive nuclide. When the imaging moiety is a radioactive metal ion, i.e. a radiometal, suitable radiometals can be either positron emitters such as ⁶⁴Cu, ⁴⁸V, ⁵² Fe, ⁵⁵Co, ⁹⁴mTc or ⁶⁸Ga or γ-emitters such as ⁹⁹mTc, ¹¹¹In, ¹¹³In, ⁶⁷Ga. When the imaging moiety is a positron-emitting radioactive non-metal, suitable such positron emitters can include: ¹¹c, ¹³N, ¹⁵O, ¹⁷F, ¹⁸F, ⁷⁵Br, ⁷⁶Br or ¹²⁴I.

The term “biocompatible”, as used herein, refers to a material that along with any metabolites or degradation products thereof that are generally non-toxic to the recipient and do not cause any significant adverse effects to the recipient and do not adversely affect the short-term viability or long-term proliferation of a target biological particle within a particular time range. Generally speaking, biocompatible materials are materials that do not elicit a significant inflammatory or immune response when administered to a patient.

The term “administration” refers to introducing an agent (or a compound including the agent, where the agent can be a phosphorescent imaging probe, for example) of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used. In an embodiment, the agent is administered locally (e.g., colon) so that it is not systemically distributed throughout the body.

In accordance with the present disclosure, an “effective amount” or “a detectably effective amount” of the agent (e.g., a pH-triggered phosphorescent imaging agent, such as a CuInSe₂ nanotube) of the present disclosure is defined as an amount sufficient to yield a discernable, acceptable image using equipment that is available for pre-clinical or clinical use. In an embodiment, a detectably effective amount of the agent of the present disclosure may be administered in more than one injection. The detectably effective amount of the agent of the present disclosure can vary according to factors such as the degree of susceptibility of the individual, the age, sex, and weight of the individual, idiosyncratic responses of the individual, the dosimetry, and the like. Detectably effective amounts of the agent of the present disclosure can also vary according to instrument and digital processing related factors. Optimization of such factors is well within the level of skill in the art.

As used herein, the term “subject” includes humans, mammals, and birds (e.g., mice, rats, pigs, cats, dogs, birds, and horses,). Typical subjects to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to host or organisms noted above that are alive. The term “living subject” refers to the entire host or organism and not just a part excised (e.g., a liver or other organ) from the living subject.

As used herein, the terms “optional” or “optionally” means that the subsequently described event or circumstance can or cannot occur, and that the description includes instances where said event or circumstance occurs and instances where it does not.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

### DISCUSSION

In accordance with the purpose(s) of the present disclosure, as embodied and broadly described herein, embodiments of the present disclosure, in some aspects, relate to imaging probes including CuInX₂ nanotubes (where X is a chalcogen selected from S, Se, and Te), such as, but not limited to CuInSe₂ nanotubes, where the nanotubes are capable of experiencing a large Stokes shift in emission intensity in response to relatively small changes in environmental pH. Aspects of the present disclosure also include methods of imaging, such as a tissue (e.g., In vitro or in vivo in a human or animal subject) with the CuInSe₂ nanotube imaging probes of the present disclosure. Embodiments also include methods of imaging cancer (e.g., tumors) in a subject with the CuInSe₂ nanotube imaging probes.

Optical bioimaging in the second near-infrared (NIR-II; 1000-1700 nm) window has attracted great attention in life science owing to large penetration depth and high spatial resolution. As a result, in the past few years there has been growing interest in designing various types of probes such as small organic molecules, semiconducting quantum dots and rare-earth nanocrystals, which has led to a boom of the library of NIR-II bioimaging probes. From a photophysical point of view, thus far all the previously reported NIR-II probes only produce fluorescent signal. Fluorescent probes are inherently subject to multiple drawbacks, including small Stokes shift as exemplified by two representative probes (about 50 nm for organic cyanine-involved fluorophores and below 200 nm for inorganic IV-VI nanocrystals)^{7,8} and short luminescent lifetimes (nanosecond scale similar to that of tissue autofluorescence)⁹. The autofluorescence signals out of focus in an epifluorescence microscope, creating a “white stars in a black sky” effect¹⁰, which will superimpose onto the in-focus NIR-II signal of interest and thus blur the images. Despite intriguing in NIR-II in vivo imaging technology, how to achieve clear and deep target-specific imaging in the dynamic physiological environments is still a formidable challenge.

As a key type of photoluminescence (PL) related to fluorescence, room temperature phosphorescence has aroused interest in the fields of photonics and organic electronics because of its long-lived luminescence, large Stokes shift and high signal-to-noise ratio^{11,12}. The functional integration of benefits from phosphorescence and NIR-II bioimaging modality may provide a new frontier of NIR-II phosphorescent imaging, may overcome the drawbacks inherent to traditional NIR-II fluorescent imaging, and could offer a diverse powerful technique for high-quality bioimaging that potentially help to address important unmet medical needs. Unfortunately, mainly due to to currently inadequate probe design strategy, achievement of NIR-II phosphorescent imaging has not yet been established.

The probes, compounds, compositions, and methods of the present disclosure employ a physiological pH-induced radiative mode switching strategy to provide NIR-II phosphorescent nanoprobe. As described in the examples below, glutathione (GSH)-stabilized CuInSe₂ nanotubes according to the present disclosure displayed almost no fluorescence in neutral pH conditions, but were sharply illuminated via NIR-II phosphorescence at about 1130 nm in response to a small change in pH from neutral to slightly acidic, such as the slightly acidic extracellular environment produced by cancerous tumors. This is believed to be the first example of in vivo probes that efficiently emit NIR-II phosphorescence

Thus, the present disclosure, in embodiments, provides pH-sensitive NIR-II phosphorescent imaging probes, methods of using the probes to image a subject (e.g., tissue in a subject), methods of imaging cancer, methods of making the imaging probes, and the like. Embodiments of imaging probes of the present disclosure include CuInX₂ nanotubes, where X is a chalcogen selected from S, Se, and Te. While the discussion and examples below relate primarily to CuInSe₂ nanotubes, nanotubes or nanostructures made from other calcogens such as S and Te can have similar properties and provide similar functionalities as the CuInSe₂ nanotubes described herein. One of skill in the art will appreciate that in chemical preparation, S, Se, and Te can typically be replaced in synthesis. Thus, the intent is to cover variations of the CuInSe₂ nanotubes in which the Se is replaced with S or Te.

In embodiments, imaging probes of the present disclosure include CuInX₂ nanotubes, such as CuInSe₂ nanotubes, where the nanotubes have an outer diameter and an inner diameter defining a hollow center. These CuInSe₂ nanotubes have the unique ability to emit a weak florescence (barely detectable to the naked eye) at a neutral pH of about 7.0 or higher (e.g., more basic, 7.0-14). Notably, biological pH (e.g., blood) is about 7.2. Then, the CuInX₂ CuInSe₂ nanotubes shift to emit strong, detectable NIR-II phosphorescence at a pH lower than 7.0 (e.g., more acidic, such as 6.9-0.1). In embodiments, CuInSe₂ nanotubes of the present disclosure shift to emit strong, detectable NIR-II phosphorescence at a pH of about 6.9 or lower, such as about 6.87 or lower, about 6.8 or lower, and so on. As used herein in reference to pH, “lower” refers to lower numbers on the pH scale, e.g., more acidic, and “higher” refers to higher numbers on the pH scale, e.g., more basic, with 7.0 being neutral). This is useful in that certain biological conditions (e.g., cancer) are associated with a lowered, slightly acidic extracellular pH as compared to normal, healthy tissues. The CuInSe₂ nanotubes of the present disclosure also have the ability, not only to be “turned on” when the environmental pH drops to about 6.9 or lower, but also to be turned back “off” when the environmental pH rises to about 7.0 or higher, since the phosphorescence will decrease when the pH rises above about 6.9.

In embodiments, the CuInSe₂ nanotubes or the present disclosure each comprise a ligand/capping moiety. In embodiment, the ligand/capping moiety is glutathione (GSH). As described in greater detail below, the GSH moieties perform various functions in the nanotubes, such as capping, chelating, and self-assembly of the nanotube aggregates. In addition to GSH, any molecules having thiol (—SH), amino groups (—NH2) and carboxyl groups (—COOH) can perform a similar function of GSH. For example, compounds, such as, but not limited to, cysteine may be used in place of GSH to provide a ligand/capping/chelating function.

In embodiments, the nanotube has an outer diameter of about 5 nm to about 20 nm, such as an outer diameter of about 10-15 nm, about 12-14 nm, about 11.5 nm, and the like. In embodiments, the nanotube has an inner diameter (also representing the diameter of the hollow space) of about 2 nm to about 10 nm. The nanotube shell can have a thickness (e.g. the thickness of the nanotube between the outer diameter and inner diameter) of about 2 nm to about 8 nm, such as about 4.1 nm. In embodiments the nanotubes can have a length of up to hundreds of nanometers, e.g. about 50-500 nm, about 100-200 nm, about 170 nm, and the like. In embodiments, the nanotube has an outer portion near the outer diameter an inner portion near the hollow center that differ in chemical composition. In embodiments, the chemical composition of the outer portion is predominately CuInSe₂, and the composition of the inner portion includes more In₂Se₃ nanoparticles than the outer portion.

In embodiments, the CuInSe₂ nanotube emits phosphorescence in the second NIR range at about 1000-1700 nm. For instance, in embodiments, the nanotubes emit phosphorescence at about 1130 nm at a pH of about 6.5 to about 6.8. The nanotubes of the present disclosure experience a large change in emission intensity with a corresponding small change in pH. For instance, in embodiments, the CuInSe₂ nanotube experiences a Stokes shift in emission intensity of about 424 nm over a change in pH of only about 0.4. The imaging probe of the present disclosure also has the advantage of being able to switch between an “on” (e.g., phosphorescent) and “off” (little to no detectable phosphoresce) mode. Thus, photoluminescent activation of the nanotube imaging probes is reversible such that the photoluminescence of the CuInSe₂ nanotube decreases at a pH above about 6.87-7.0 and can then be “re-activated” if the pH shifts back to a lower/acidic pH.

In embodiments, the CuInSe₂ nanotubes of the present disclosure have an atomic ratio of cooper:indium:selenium for about 3.66:0.39:2 to 0.68:1.29:2, such as about 0.72:1.01:2.

The present disclosure also includes compositions, such as pharmaceutical imaging compositions including a plurality of imaging probes of the present disclosure. Embodiments of the present disclosure also include pharmaceutically acceptable imaging compositions comprising a plurality of imaging probes of the present disclosure described above and a pharmaceutically acceptable carrier. Pharmaceutical compositions of the present composition can include a detectably effective amount of the imaging probes of the present disclosure (the “active ingredient”) and one or more pharmaceutically acceptable carriers or diluents.

In practice, the compounds of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired imaging effect in association with the pharmaceutical carrier. That is, a “unit dosage form” is taken to mean a single dose wherein all active and inactive ingredients are combined in a suitable system, such that the patient or person administering the composition to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Methods of the present disclosure include various methods of using the imaging probes of the present disclosure. For instance, the present disclosure also includes methods of generating an image of a tissue in an animal or human subject by administering to an animal or human subject a pharmaceutically acceptable composition including a plurality of the CuInX₂ nanotubes, such as CuInSe₂ nanotubes, of the present disclosure. In embodiments, a detectably effective amount is administered to the subject. The preferred pharmaceutical composition for the present disclosure is a liquid formulation, such as, but not limited to a liquid formulation for intravenous or parenteral administration.

The CuInSe₂ nanotubes are useful as imaging probes because they emit weak, nearly undetectable fluorescence at an environmental pH of about 7.0 or higher. However, as described in much greater detail in the example below, the nanotubes experience a drastic shift in emission spectra with a small lowering of pH. The CuInSe₂ nanotubes of the present disclosure form nanoaggregates in tissues (particularly tissues featuring an acidic extracellular environment) and emit NIR-II phosphorescence in a second near-infrared range of about 1000-1700 nm at an environmental pH of about 6.87/6.8 or lower. Methods of imaging include obtaining an image of the location of nanoaggregates of the CuInSe₂ nanotubes in a tissue of the animal or human subject by detecting and imaging the phosphorescence.

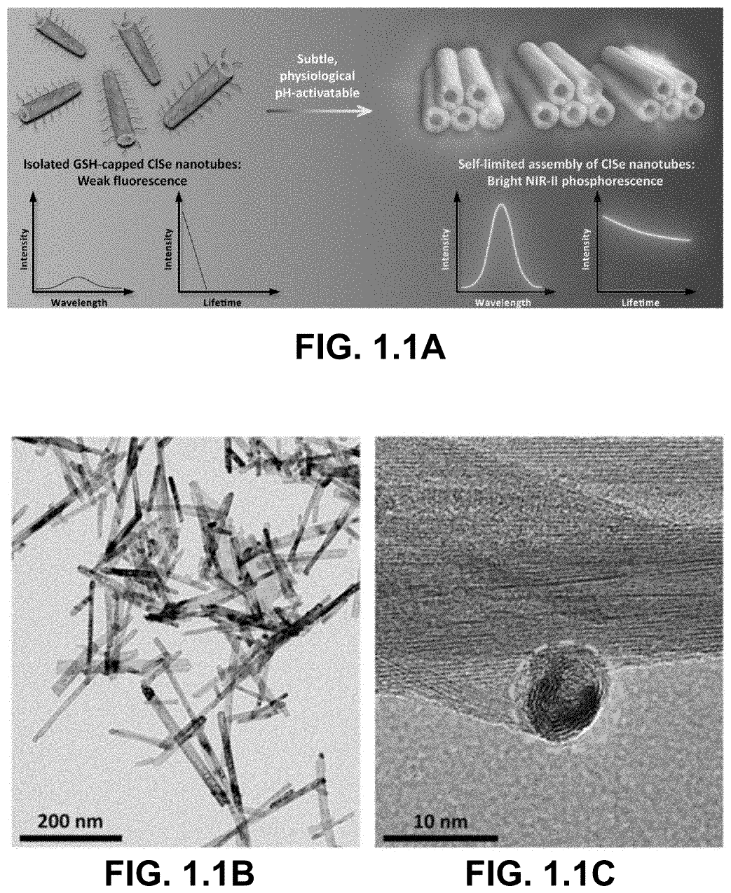

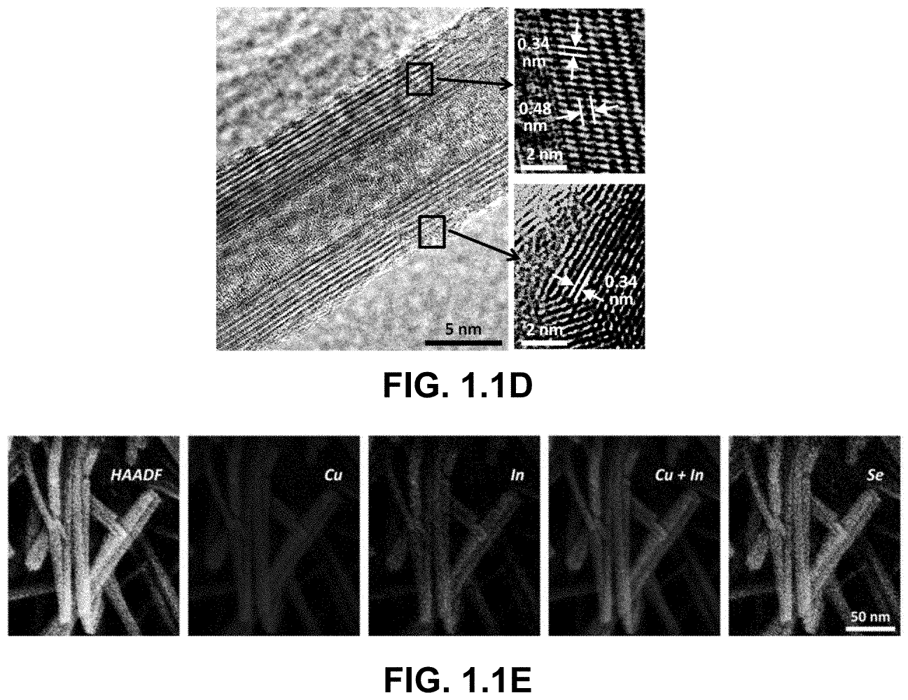

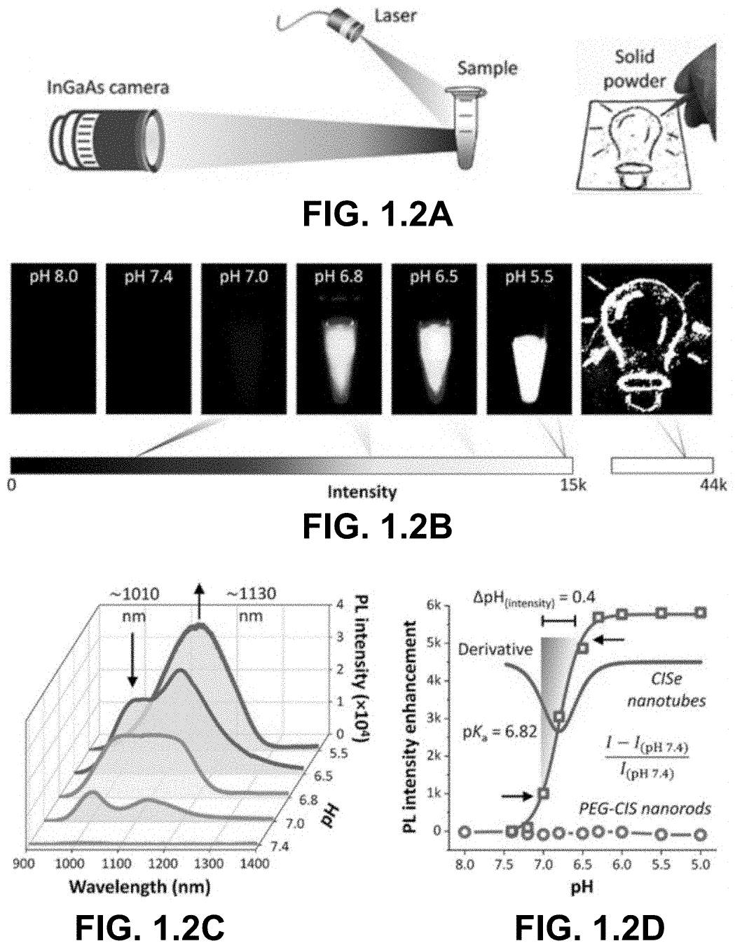

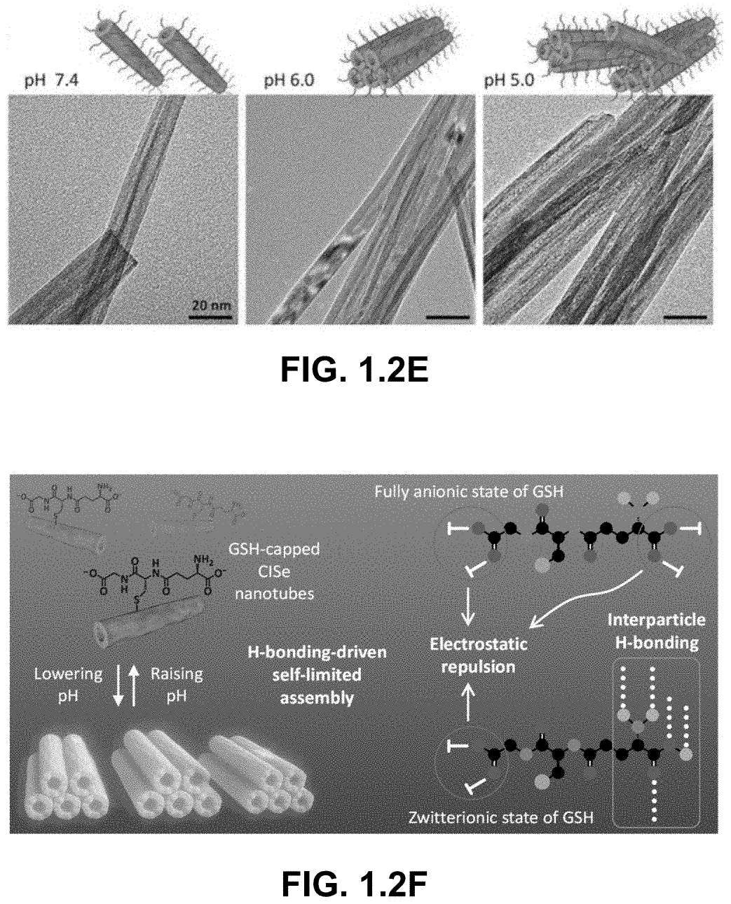

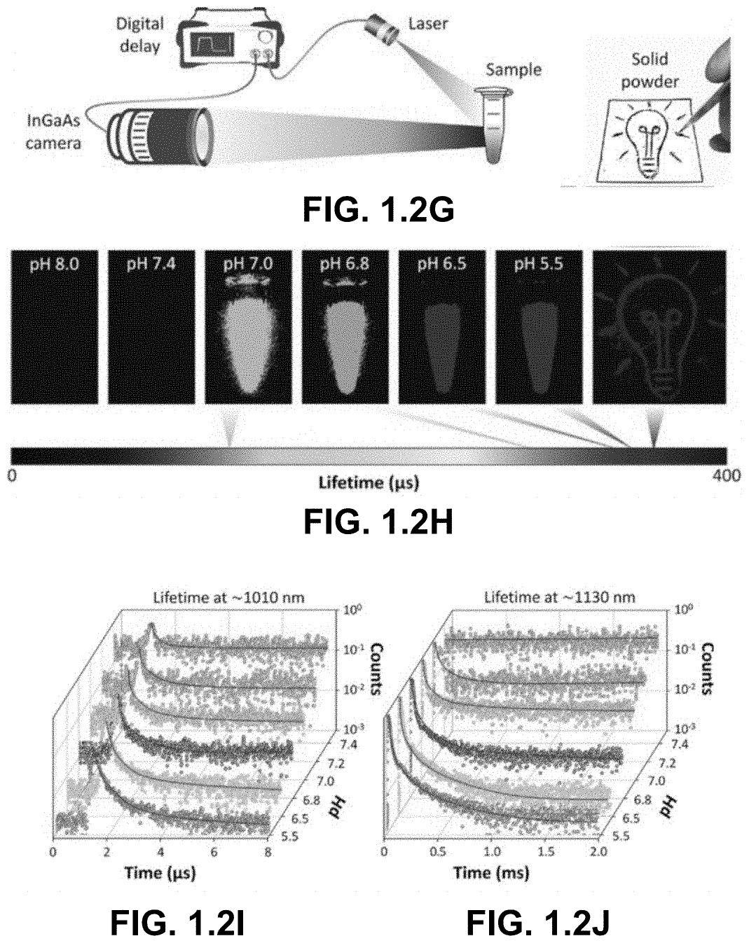

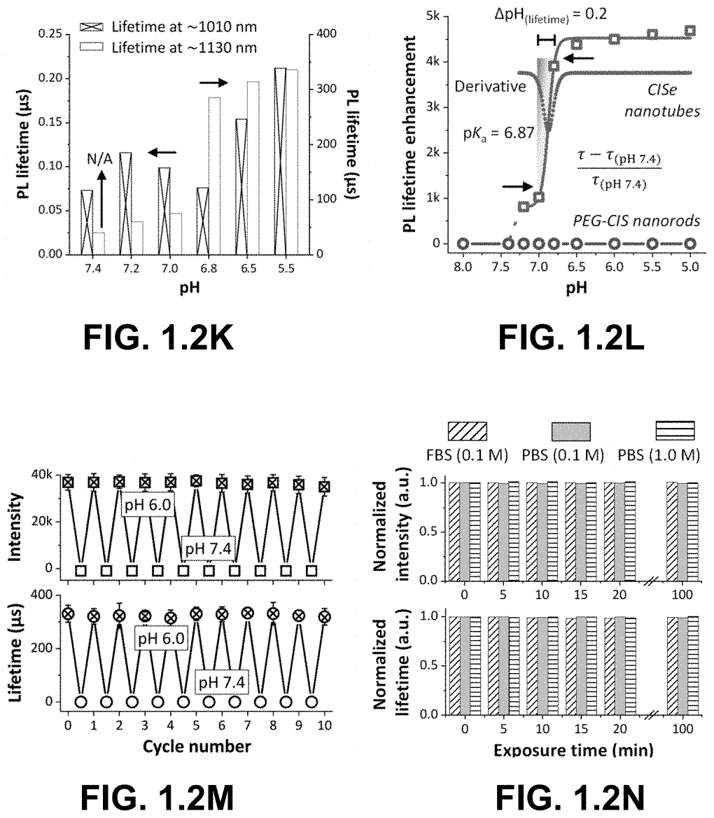

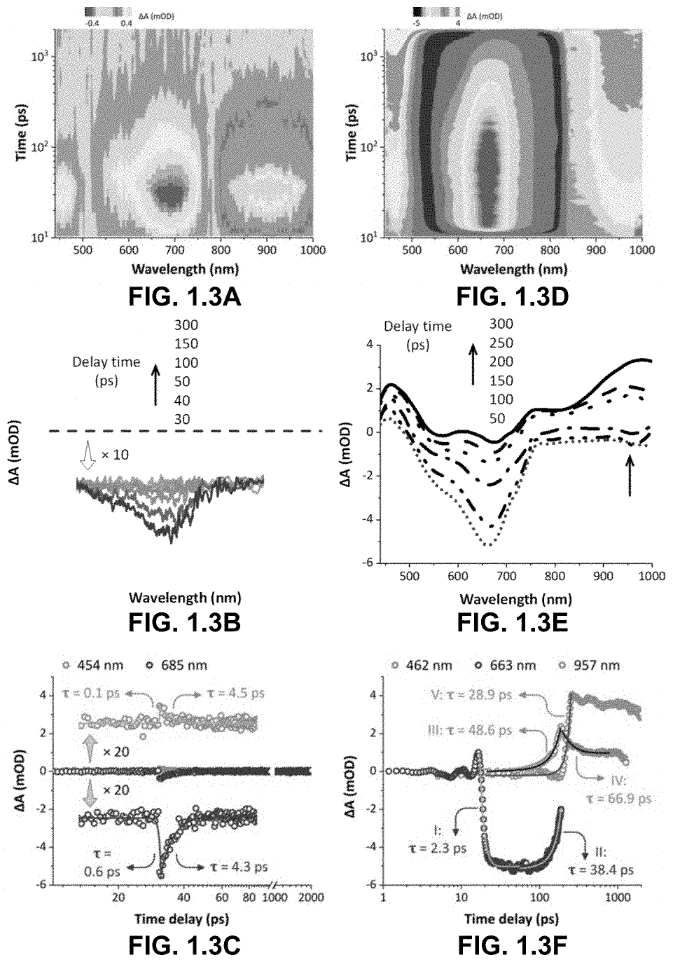

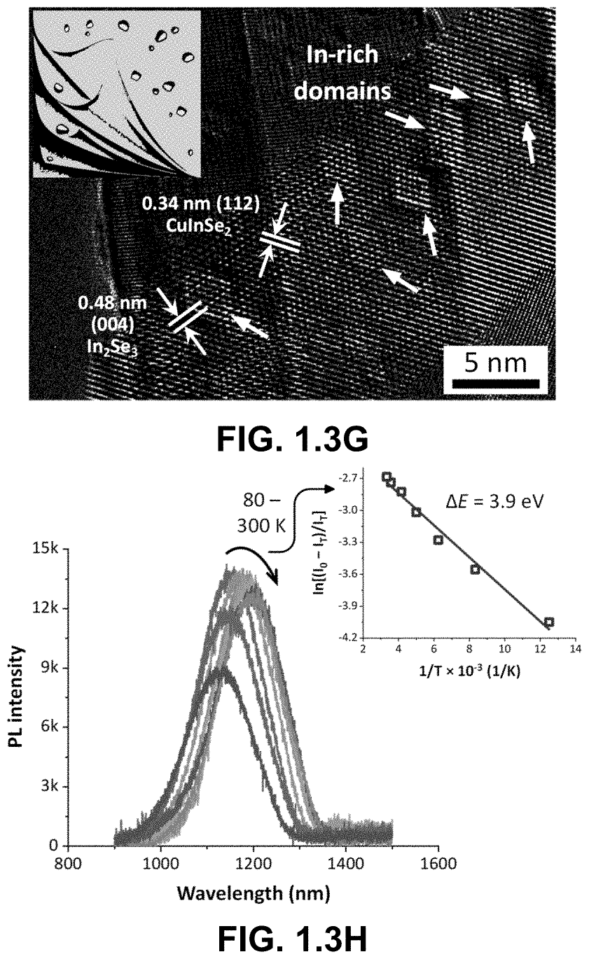

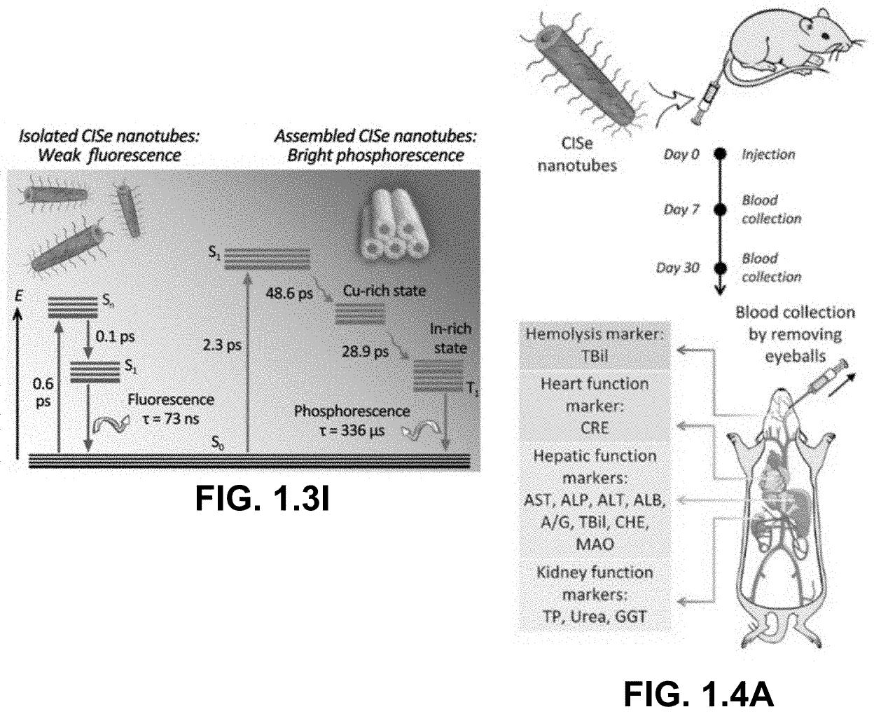

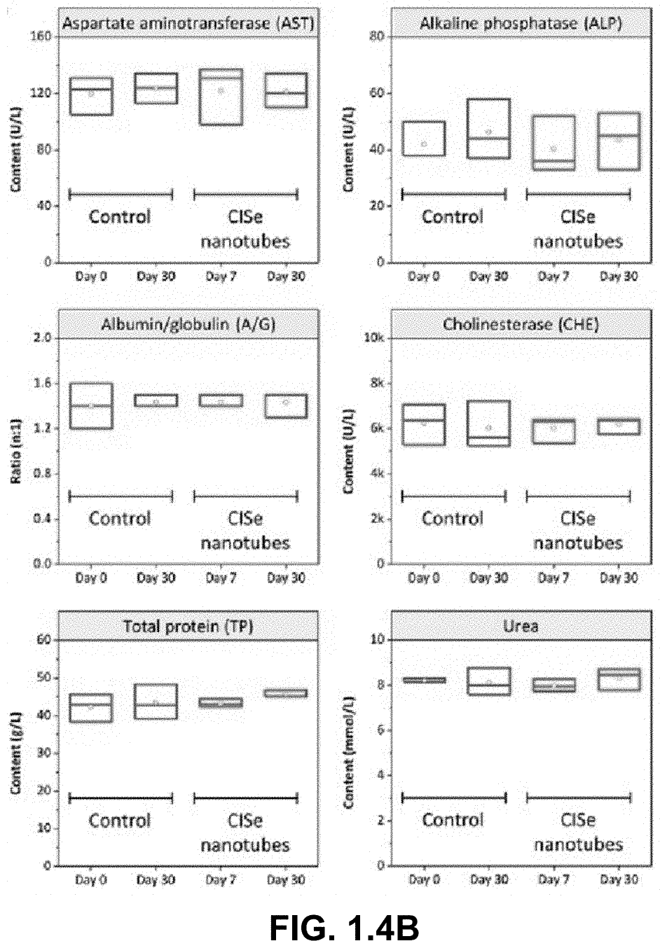

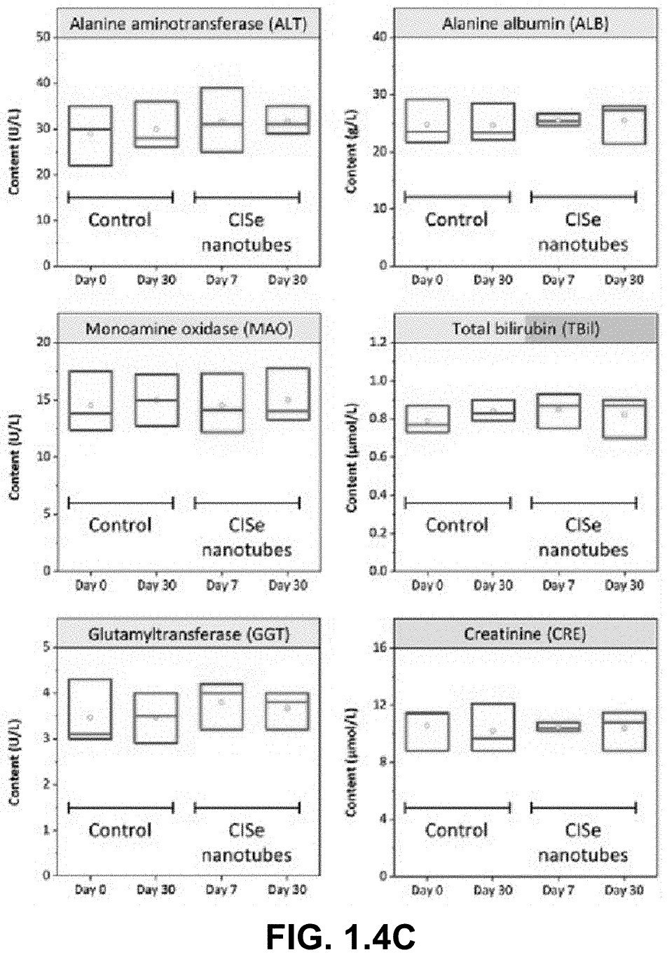

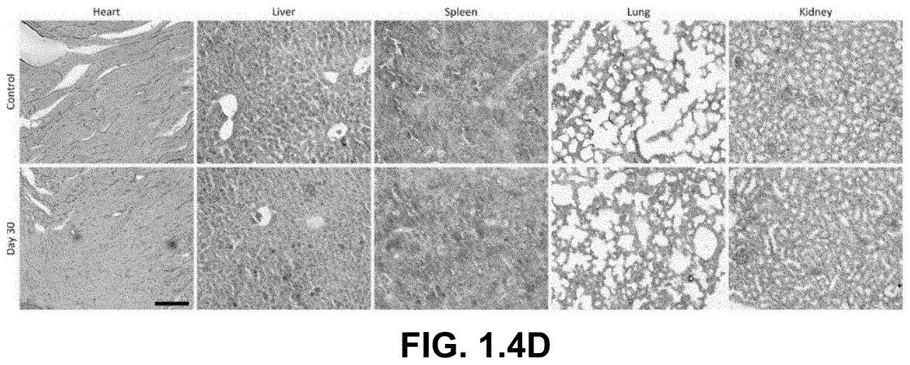

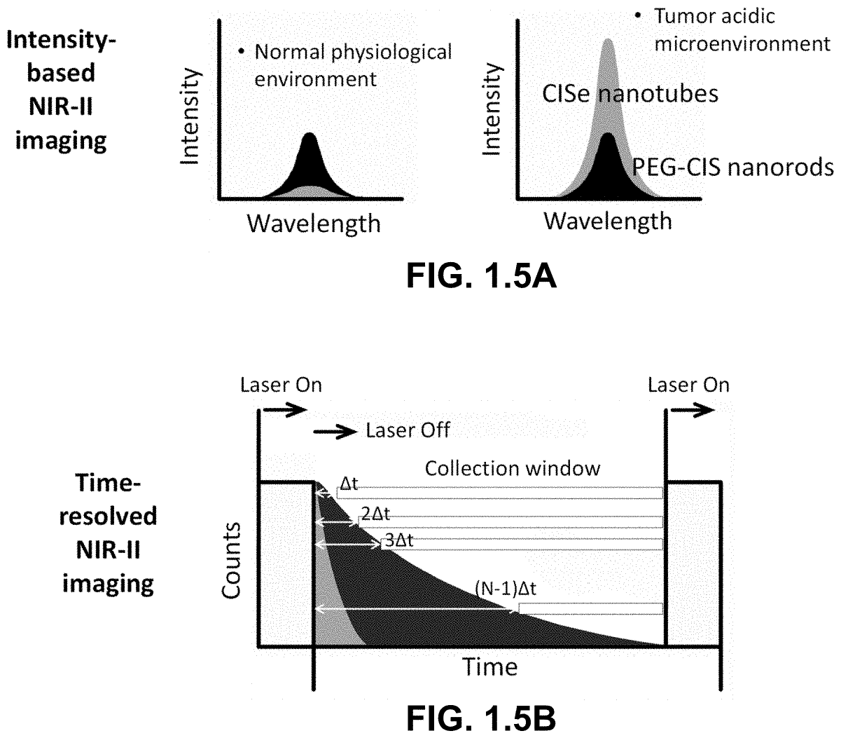

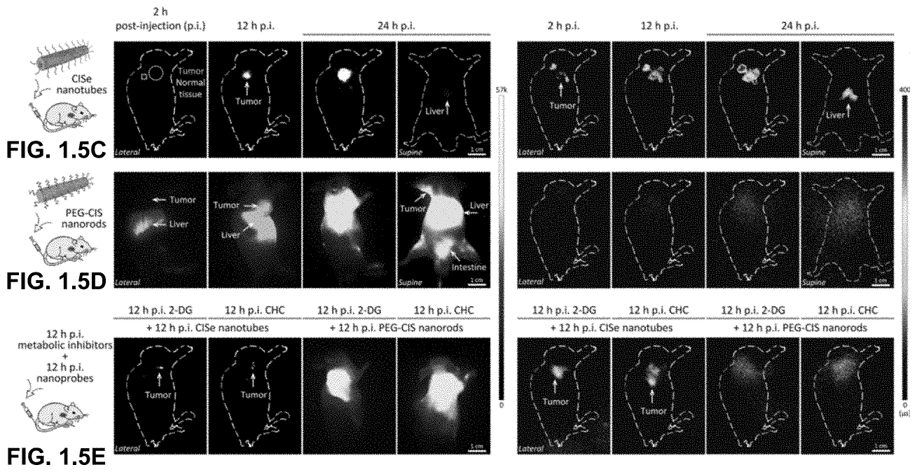

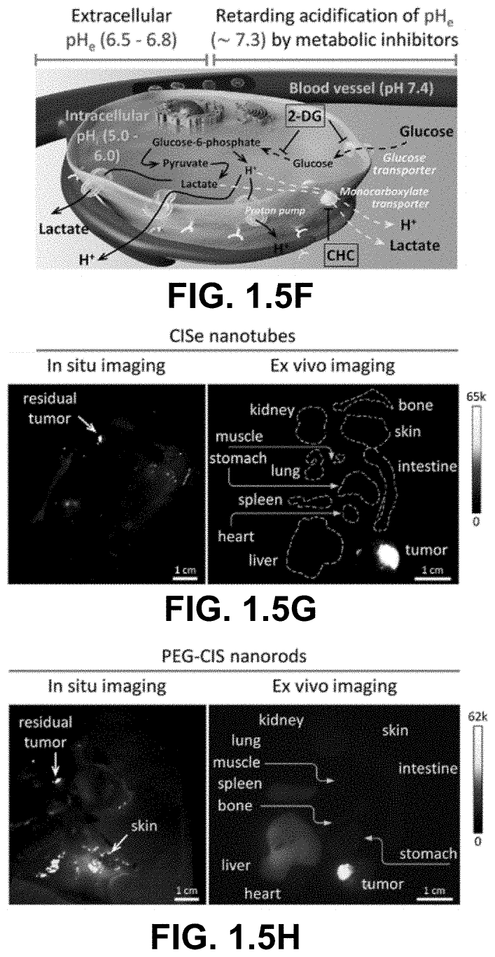

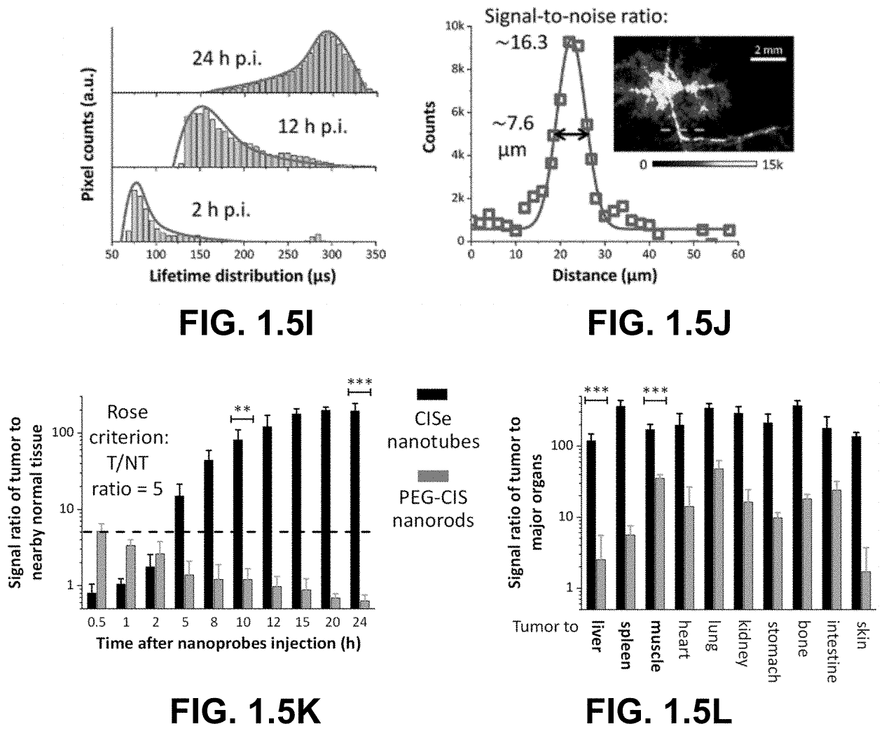

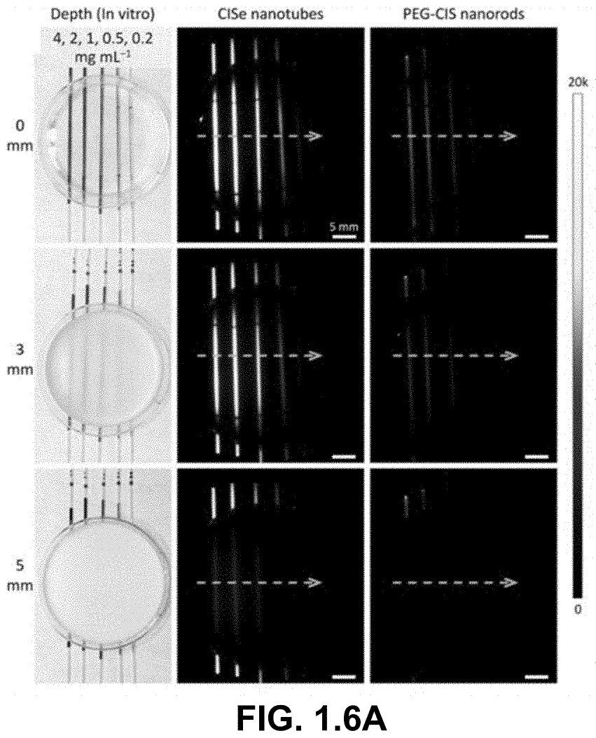

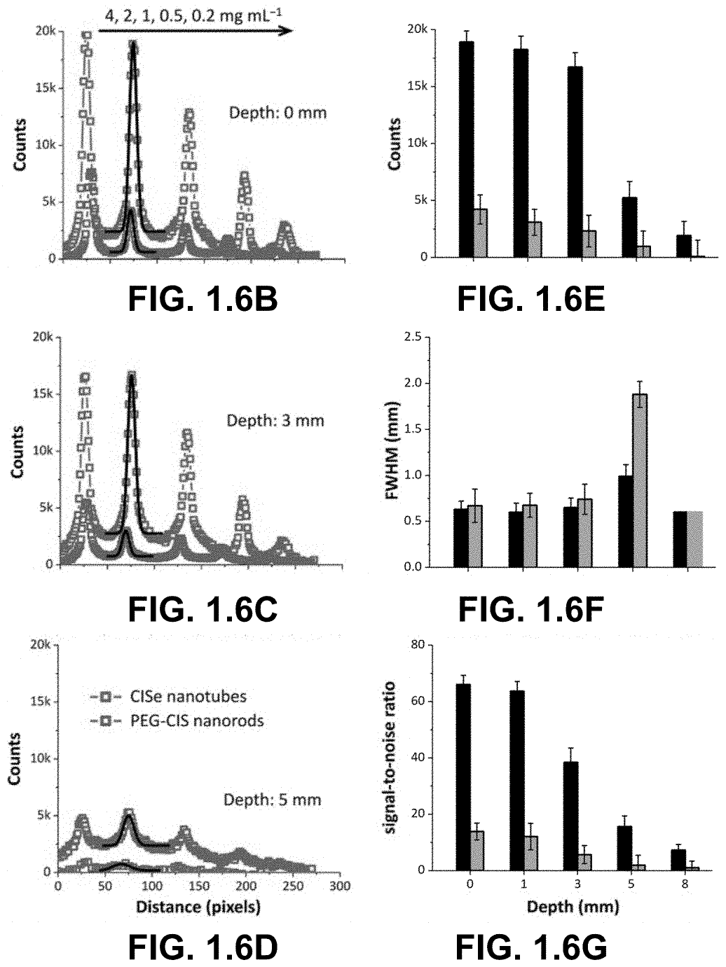

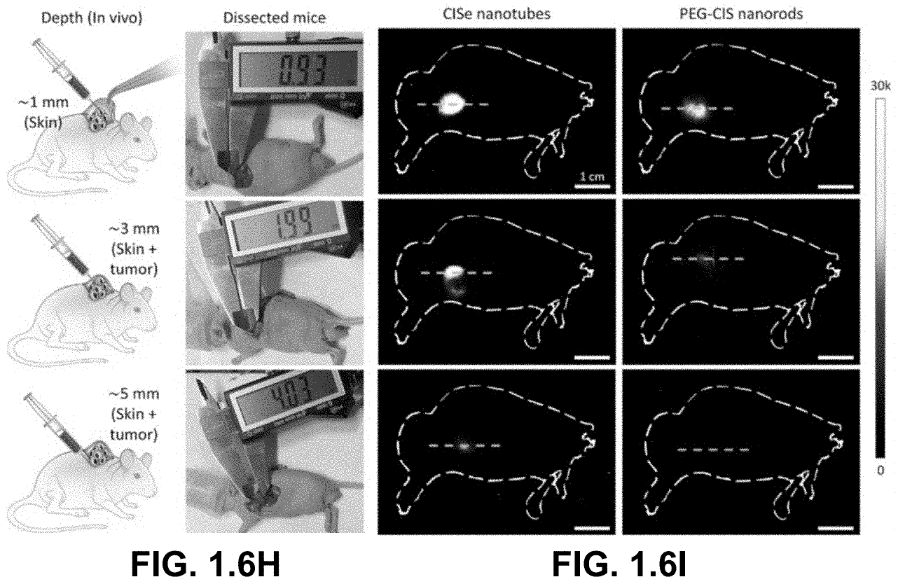

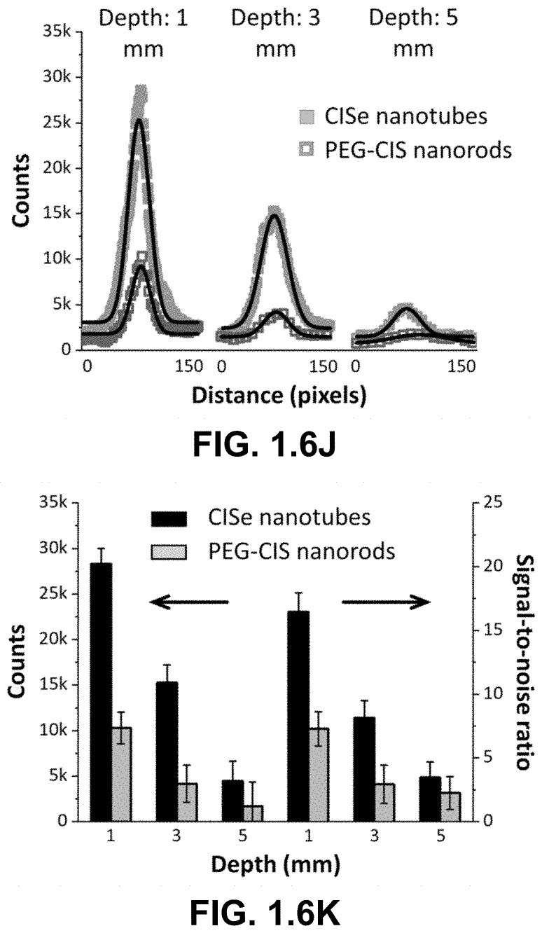

In embodiments, the CuInSe₂ nanotubes aggregate and produce a detectable phosphorescent signal at about 1000 nm or higher in tissues having an extracellular pH of about 6.8 or lower. Such tissues can include cancerous tissue. In embodiments, the CuInSe₂ nanotube emits phosphorescence at about 1130 nm at a pH of about 6.5 to about 6.8. As described in the example below, the CuInSe₂ nanotubes can produce a Stokes shift in emission intensity of about 424 nm over a change in pH of as low as about 0.4. In embodiments, the image of the location of nanoaggregates of the CuInSe₂ nanotubes is obtained with an imaging system configured to detect phosphorescence in a second near infrared range of about 1000-1700 nm. In embodiments, the imaging system is a time-resolved NIR-II imaging system, which can produce with adjustable delay time and exposure time. Example such NIR-II imaging systems are described in the examples below and illustrated in FIG. 1.5B. In embodiments of an NIR-II imaging system, a digital delay (SRS Co. Ltd., DG535) was used to generate two level signals. One signal was connected with a continuous wave laser to produce pulsed light source. Another signal was used to trigger NIR detector (charge-coupled device, CCD) with adjustable delay time and exposure time. Images with desired delay time were captured by CCD. In order to compose lifetime images, a series of N time-gated images were captured with an arithmetic sequence of time delays (0, Δt, 2Δt, (N−1)Δt). For each pixel, its intensity was extracted from above time-gated images to form a discrete PL decay curve with sampling interval of Δt. After that, the lifetime can be calculated for each pixel through the Method of Successive Integration, which treated the PL decay profiles as a single-exponential function. In embodiments, the selected single-exponential fitting was sufficient to discriminate long-lived phosphorescence from autofluorescence

Due to the slightly acidic environment of many cancerous tumors (due to multiple reasons, as explained in more detail below, such as inadequate vasculature and inefficient drainage of waster products, etc.) methods of the present disclosure can include in vivo imaging of cancer in an animal or human subject. In embodiments, the methods include administering to an animal or human subject a pharmaceutically acceptable composition comprising a plurality of CuInX₂ nanotubes, such as CuInSe₂ nanotubes, (e.g., in a detectably effective amount), such that the CuInSe₂ nanotubes emit weak fluorescence at an environmental pH of about 7.0 or higher and form nanoaggregates and emit NIR-II phosphorescence in a second near-infrared range of about 1000-1700 nm in cancerous tissue having an environmental pH of about 6.8 or lower. The methods also include obtaining an image of the location of nanoaggregates of the CuInSe₂ nanotubes in a tissue of the animal or human subject by detecting and imaging the phosphorescence, where the location of nanoaggregates of the CuInSe₂ nanotubes indicates the location of a tumor.

As described in greater detail in the Example below, in embodiments the tumor-to-normal-tissue (T/NT) signal ratio for CuInSe₂ nanotubes is above about 5, enabling distinction between healthy tissue and cancerous tissue. While 5 is about the minimum signal to noise ratio for detection, in embodiments using the imaging probes of the present disclosure, the tumor-to-normal-tissue (T/NT) signal ratio for CuInSe₂ nanotubes is from about 180 to about 200 at about 24 hours post administration which is far greater than currently available fluorescent probes. In embodiments, the tumor-to-normal-tissue (T/NT) signal ratio for CuInSe₂ nanotubes is greater than the T/NT signal ratio for Ag₂S nanoparticles.

It is also important to be able to distinguish positive signal from clearance organs such as liver, bladder, etc. to avoid background signal from these organs. In embodiments, the tumor-to-liver (T/L) phosphorescent signal ratio for CuInSe₂ nanotubes is about 170 to about 150.

Embodiments of the present disclosure also include a system for generating an image of a tissue in an animal or human subject according to the methods of the present disclosure. In embodiments, such an imaging system includes a pharmaceutically acceptable imaging composition comprising a plurality of imaging probes of the present disclosure and a pharmaceutically acceptable carrier and an imaging system configured to detect phosphorescence in a second near infrared range of about 1000-1700 nm. Embodiments of the present disclosure also include kits including CuInX₂ nanotubes, such as CuInSe₂ nanotubes, of the present disclosure, a pharmaceutically acceptable carrier or instructions for combining the nanotubes with a pharmaceutically acceptable carrier in a pharmaceutically effective amount and instructions for use (e.g., instructions for administering to a subject and/or instructions for use of an imaging system to create an image of a tissue in a subject with the nanotubes of the present disclosure.

Embodiments of the present disclosure also include methods of making the CuInX₂ nanotubes, such as CuInSe₂ nanotubes, of the present disclosure. In embodiments, the method includes the steps of: (a) synthesizing Cu₂₋ₓSe solid nanorods by water-evaporation-induced self-assembly; and (b) reduction of the Cu₂₋ₓSe nanorods with NaBH₄ to form hollow CuInSe₂ nanotubes. In embodiments, step a) comprises combining Se powder with glutathione (GSH), in basic solution under heat and combining with Cu(NO₃)₂ aqueous solution and heating to produce Cu₂₋ₓSe solid nanorods. In embodiments, step b) comprises combining the Cu₂₋ₓSe solid nanorods from step a) with aqueous InCl₃ and NaBh₄ solution and autoclaving at about 150-250° C., such as about 210° C., to produce CuInSe₂ nanotubes.

Although the methods described herein are described primarily with reference to in vivo imaging in a subject, one of skill in the art will also appreciate that the imaging probes of the present disclosure can also be used in methods of generating pH responsive image (In vitro or in vivo in a tissue or other medium).

It is also contemplated that due to the accumulation of the imaging probes of the present disclosure in tumors and other cancerous tissue, and the hollow configuration of the probes, that the probes could also be used as a delivery vehicle for an active agent (e.g., chemotherapeutic agent, and the like).

Additional details regarding the methods, compositions, and organisms of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1—NIR-II Phosphorescent Imaging Techniques and CuInSe₂ Nanotube Probes

Fluorescence bioimaging in the second near-infrared window (NIR-II; 1000-1700 nm) is a greatly promising and actively studied technique, whereas bioimaging with NIR-II phosphorescence has not been explored. The present example demonstrates, for the first time, that NIR-II phosphorescence holds vast potential for high-quality tumor imaging because of its long-lived lifetime, large Stokes shift and autofluorescence-free photophysical features. The present example demonstrates that, as the first NIR-II phosphorescent probe, CuInSe₂ nanotubes can efficiently switch the radiative mode from extremely faint fluorescence to strong NIR-II phosphorescence upon receiving subtle triggers in physiologically relevant pH scope (ΔpH<0.4), showing a Stokes shift of 430 nm, 6.3×10⁴ and 5.8×10³-fold increases in emission lifetime and intensity, respectively. Supersensitive, nonlinear amplification of the NIR-II phosphorescent signal at the tumor site with noise-free background enabled CuInSe₂ nanotubes to exhibit ultrahigh tumor-specific imaging with excellent tumor-to-liver ratio of 110 and tumor-to-normal tissue ratio of 190 after 24 h post-injection. Moreover, CuInSe₂ (“CISe”) nanotubes allowed through-skin visualization of tumor vessels and identified an early-emerging, transient characteristic inherent to tumor vessel progression, which served as a basis to assess enhanced permeability retention (EPR) effect, a controversial topic in nanomedicine. The use of NIR-II phosphorescence represents a shift in the imaging paradigm and brings new opportunities to address major medical challenges.

### Materials and Methods

Synthesis. For all synthesis details, please see Example 2.

Intensity-based NIR-II imaging system. Briefly, all the NIR-II images were captured on a 640×512 pixels two-dimensional InGaAs array (Princeton Instruments, 2D InGaAs focal plane array) utilizing a UniNano® NIR-II system. The excitation was generated using an 808 nm diode laser by an optical fiber and collimator. Emission was collected with a 1000 nm long-pass optical filter (0.14 W cm⁻² of laser power density). A lens set was used to obtain tunable magnifications ranging from 1× (whole body) to 10× (high magnification) by changing the relative position of two NIR achromats (75 mm and 200 mm, Thorlabs). Image J software was utilized for analyzing the images.

Time-resolved NIR-II imaging system. The purpose-built time-resolved NIR-II imaging system was illustrated in FIG. 1.5B. The digital delay (SRS Co. Ltd., DG535) was used to generate two level signals. One signal was connected with a continuous wave laser to produce pulsed light source. Another signal was used to trigger NIR detector (charge-coupled device, CCD) with adjustable delay time and exposure time. Images with desired delay time were captured by CCD. In order to compose lifetime images, a series of N time-gated images were captured with an arithmetic sequence of time delays (0, Δt, 2Δt, (N−1)Δt). For each pixel, its intensity was extracted from above time-gated images to form a discrete PL decay curve with sampling interval of Δt. After that, the lifetime can be calculated for each pixel through the Method of Successive Integration, which treated the PL decay profiles as a single-exponential function. Despite PL decays of CISe nanotubes and PEG-CIS nanorods actually did not follow a single-exponential manner, here the selected single-exponential fitting was sufficient to discriminate long-lived phosphorescence from autofluorescence.

Animal handling. Mouse handling was approved by Stanford University's administrative panel on Laboratory Animal Care. All experiments were performed in accordance with the National Institutes of Health Guide for the Care and Use of Laboratory Animals. Seven-week-old female C57BLJ6 and nude mice were purchased from Charles River for imaging studies and housed at the Research Animal Facility of Stanford under our approved animal protocols. Bedding, nesting material, food and water were provided. Before whole-body or tumor-specific imaging, all mice were anaesthetized in a rodent anesthesia machine with 2 L min⁻¹ O₂ mixed with 3% isoflurane. During the time course of NIR-II imaging, the mice was kept anaesthetized by a nose cone delivering 2 L min⁻¹ O₂ mixed with 3% isoflurane. Mice were randomly selected from cages for all experiments. No blinding was performed. All groups within study contained n=5 mice.

Cell culture. The cell lines including normal 3T3, MCF-7 and 143B (human osteosarcoma) cells were purchased from American Type Culture Collection (Manassas, Va.). All the cells were cultured regularly in growth medium consisting of Dulbecco's modified Eaglemedium (DMEM) supplemented with 10% FBS (fetal bovine serum, Invitrogen, Burlington, Canada) in a humidified atmosphere with a 5% CO₂ concentration at 37° C., using a 35-mm diameter plastic bottom dish (ibidi GmbH, Germany). The cells were routinely harvested by treatment with a trypsin-ethylenediaminetetraacetic acid (EDTA) solution (0.25%) (Invitrogen, Burlington, Canada).

Cell viability assay. In vitro cytotoxicity was measured by performing methyl thiazolyl tetrazolium (MTT) assays. Briefly, the normal 3T3 cells were plated in 96-well plates at a density of 5000 viable cells (100 μL of cells suspension) per well. Then, the cells were incubated with CISe nanotubes at indicated concentrations ranging from 0 to 1000 μg mL⁻¹ for 24 or 48 h at 37° C. and 5% CO₂. After that, the cells were incubated with MTT reagent at 37° C. for 3 h prior to removal of the DMEM. Upon cell lysis, the intracellular formazan product was dissolved using 100 μL DMSO and then quantified by micro-plate reader (FL600, Bio-Tek) at a wavelength of 490 nm. The relative cell growth (%) was calculated by (test/control)×100, where the control contained only cell and culture medium. The results were expressed as mean values of three measurements. The same processes of cytotoxicity of CISe nanotubes against MCF-7 cells and 143B cells were implemented as mentioned above.

In vitro metabolic studies. The metabolites (e.g., lactate secretion) were quantified by commercial enzymatic kits from Sigma-Aldrich according to the manufacturer instruction. Briefly, stock solutions of α-cyano-4-hydroxycinnamic acid (CHC, 1 M) and 2-deoxyglucose (2-DG, 1 M) dissolved in PBS were used. Then, 143B tumor cells (1.2×10⁶ per well) were seeded in 6 well plates and incubated for 24 h before inhibition studies. Media were removed and replaced at the beginning of experiments with media containing inhibitors. Concentration of lactate in the culture media was subsequently measured with the MAK064 Assay Kit. Protein concentrations were analyzed from cell pellets using a Pierce BCA protein assay kit. Metabolic assays were conducted 6 h after inhibitor addition. The pH of the media was measured with a pH meter at selected time points after inhibitor addition.

In vitro phantom imaging studies. To compare penetration depth and resolution between NIR-II phosphorescent imaging and NIR-II fluorescent imaging, the capillaries were filled with either CISe nanotubes or PEG-CIS nanorods (0.2-4 mg mL⁻¹) in PBS (pH 5.5) and taped to the bottom of cylindrical dish. The dish was filled with different volumes of 1% Intralipid (diluted from commercial 20% stock solution). The depth of capillaries was then calculated by the dish area. The thicknesses used was 1.0, 3.0, 5.0 and 8.0 mm. After that, the NIR-II images were acquired. The depth-dependent signal intensity, feature width and signal-to-noise ratio were investigated by fitting cross-sectional intensity profiles with Gaussian function.

Xenograft tumor implantation. To set up the osteosarcoma xenograft animal model, 143B cells (roughly 5×10⁶ in 100 μL PBS) were subcutaneously injected into the right shoulder of the nude mice. To demonstrate the universal imaging applications of CISe nanotubes, another subcutaneous tumor model was developed. Specifically, 3×10⁶ MCF-7 cells were planted by subcutaneous injection into the upper rear leg of nude mice. When tumor volume of tumor-bearing mice reached ˜150 mm³ according to the formula (width²×length)/2, the mice were randomly grouped (n=5 in each group) for imaging experiments.

In vivo intensity-based NIR-II imaging. Tumor-bearing mice (about 20 g weight) were placed on a stage with venous catheter for injection. Tail vein injection of nanoprobes (0.2 mL, 2 mg mL⁻¹) was carried out in dark and synchronized with a camera that started continuous image acquisition simultaneously. As a result, the final dosage of nanoprobes (e.g., CISe nanotubes and PEG-CIS nanorods) was about 20 mg kg⁻¹. The exposure time was set as 5 ms. In addition, to validate the acidic pHₑ-activatable tumor imaging, 2-DG (dose: 250 mg kg⁻¹) or CHC (dose: 250 mg kg⁻¹) was injected 12 h before administrations of CISe nanotubes or PEG-CIS nanorods (dose: 20 mg kg⁻¹), respectively. The mice were then monitored at pre-designated time points. To further study the penetration properties of these nanoprobes in vivo, 2 mg mL⁻¹ CISe nanotubes or PEG-CIS nanorods were intratumorally injected into tumor-bearing mice at different injection sites. After NIR-II intensity imaging, the mice were carefully dissected along the injection sites. Tumor tissue was in situ cut open to identify the precise location of nanoprobes and then the injection depth was measured by a vernier caliper.

In vivo time-resolved NIR-II imaging system. Notably, the procedure was similar to the in vivo intensity-based NIR-II imaging described above.

Ex vivo NIR-II imaging. 24 h after injection of nanoprobe through tail vein, the mice were euthanized. Excised tumor and major organs including heart, liver, muscle, spleen, stomach, lung, kidney, bone, intestine and skin were imaged by intensity-based NIR-II imaging systems.

In vivo biosafety analysis. Healthy mice (n=5) were injected with 100 μL of CISe nanotubes at a dosage of 100 mg kg⁻¹. Following these injections, the mice were weighed at various time points from 0 to 30 days. Meanwhile, animal behaviors were also carefully recorded. At day 7 and 30 post-injection, the mice were anaesthetized and eyeballs were removed, followed by collection of blood samples for blood biochemistry test. The mice injected with PBS were used as the control. Subsequently, the main organs of the mice (heart, liver, spleen, lung and kidney) were harvested and fixed using 4% paraformaldehyde. Tissue samples were then embedded in paraffin, sliced (5 μm) and stained using hematoxylin and eosin (H&E). All of the obtained biopsy samples were imaged using an optical microscope (Leica).

Statistical analysis. The photoluminescence measurement was performed to quantitate NIR optical signal intensity through the Image J 1.45× software (National Institutes of Health, Bethesda, Md.). Data were given as mean±SD (standard deviation). Statistical significance was determined by a two-tailed Student's t test. A P-value of less than 0.05 was considered significant.

### INTRODUCTION

Optical bioimaging in the second near-infrared (NIR-II, 1000-1700 nm) window has attracted great attention in life science owing to large penetration depth and high spatial resolution¹⁻³. In the past few years, interest has grown in designing various types of probes such as small organic molecules, semiconducting quantum dots and rare-earth nanocrystals⁴⁻⁶, leading to a booming of the library of NIR-II bioimaging probes. From a photophysical point of view, thus far all these probes only emit NIR-II fluorescence. It is inherently subject to multiple drawbacks, including small Stokes shift as exemplified by two representative probes (about 50 nm for organic cyanine-involved fluorophores and below 200 nm for IV-VI nanocrystals)^{7,8} and short emission lifetime (nanosecond scale similar to that of autofluorescence)⁹. The autofluorescence signals out of focus in epifluorescence microscope, creating “white stars in a black sky” effect¹⁰, will superimpose onto the in-focus NIR-II signals of interest and thus blur the images. Despite intriguing in NIR-II in vivo imaging technology, how to achieve target-specific imaging clear and deep in dynamic physiological environments is still a formidable challenge.

As a key type of photoluminescence (PL) related to fluorescence, room temperature phosphorescence has previously only aroused interest in photonics and organic electronics because of its long-lived luminescence, large Stokes shift and high signal-to-noise ratio.^{11,12} This example demonstrates that these features also help overcome the drawbacks inherent to traditional NIR-II fluorescent imaging. In this context, the functional integration of benefits from phosphorescence and NIR-II bioimaging modality, ushering in NIR-II phosphorescent imaging, affords a powerful technique for high-quality bioimaging that potentially helps to address important unmet medical needs. Unfortunately, mainly owing to currently inadequate design strategy of materials, a way to achieve NIR-II phosphorescent imaging has not yet been achieved.

The present example describes a physiological pH-driven radiative mode switch strategy to develop NIR-II phosphorescent probes. Glutathione (GSH)-capped copper indium selenium (CISe) nanotubes were almost fluorescence-silent in neutral environment, and then drastically lighted their NIR-II phosphorescence up at about 1130 nm responding to slightly acidic stimuli (FIG. 1.1A). The mechanistic studies demonstrated that the emergence of NIR-II phosphorescence was ascribed to the Cu-to-In exciton transfer, in which excited state energy could be stabilized by self-limited assembly of CISe nanotubes. We were motivated to extend the feasibility and advantage of CISe nanotubes in tumor-specific imaging, which remains very challenging at present

### Results

Synthesis and Characterization of CISe Nanotubes

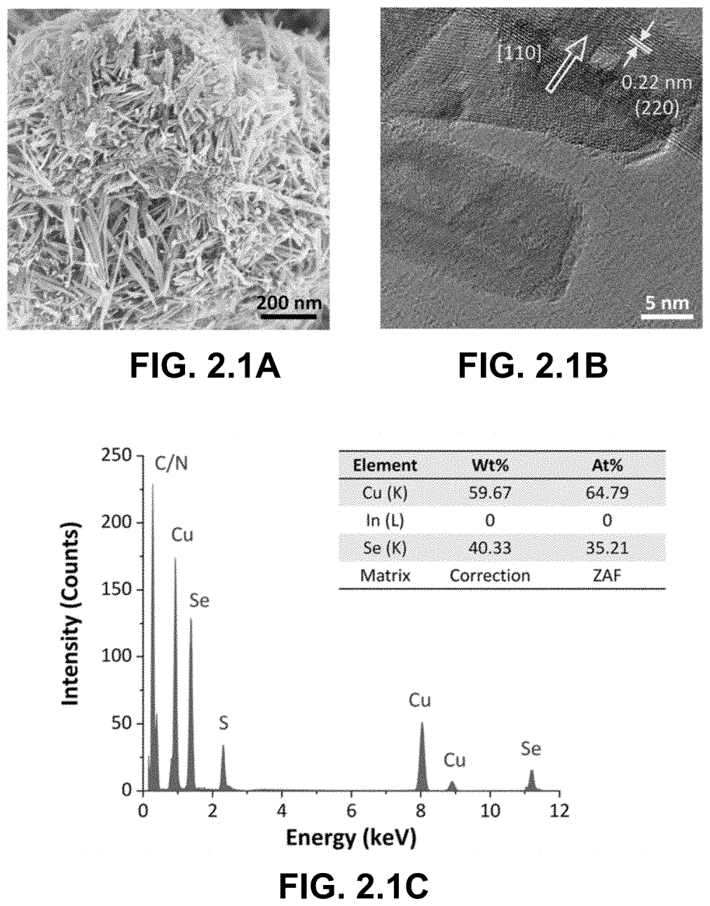

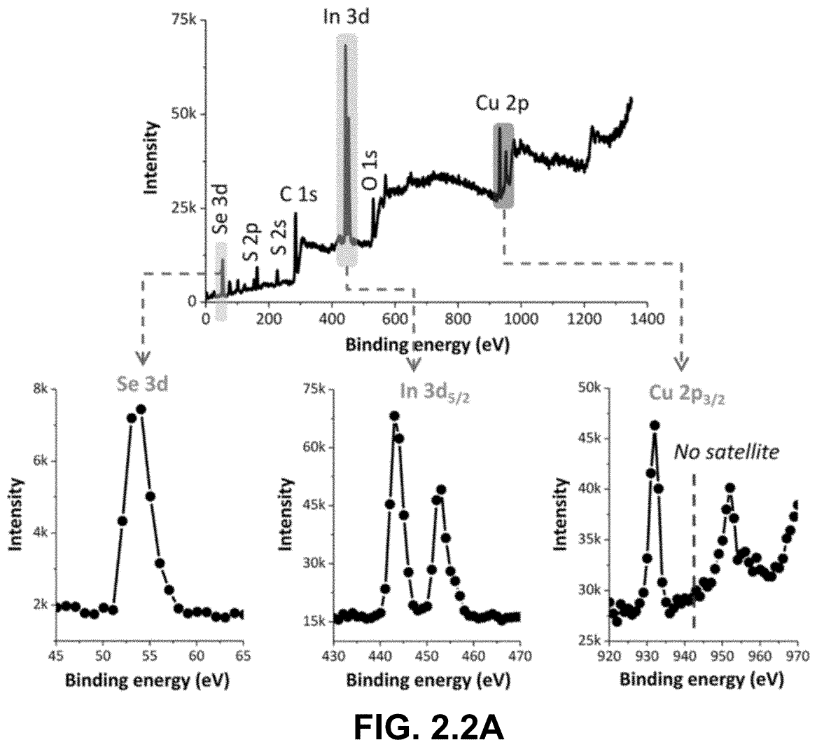

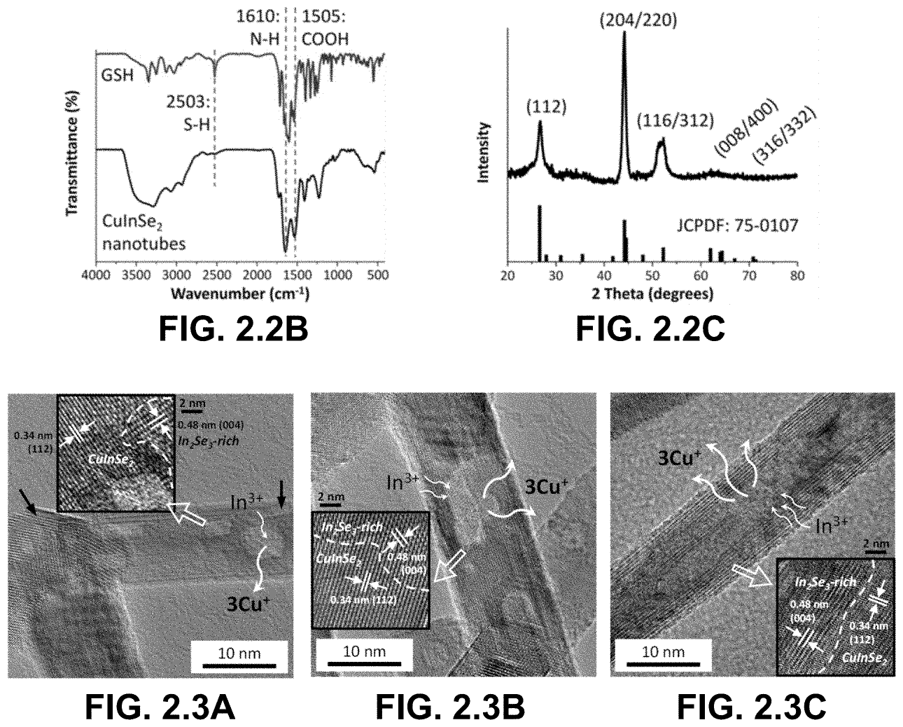

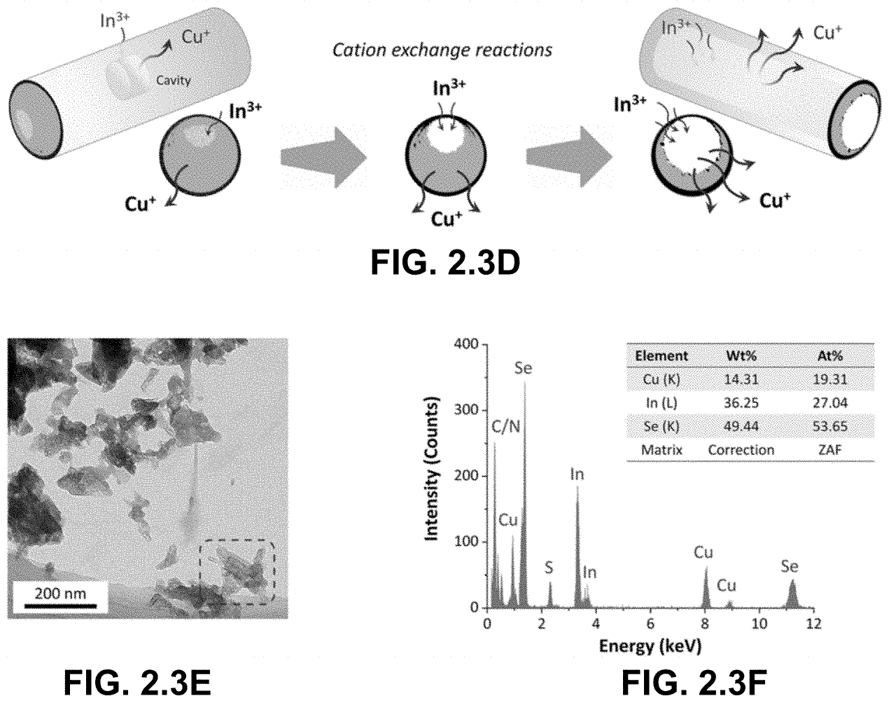

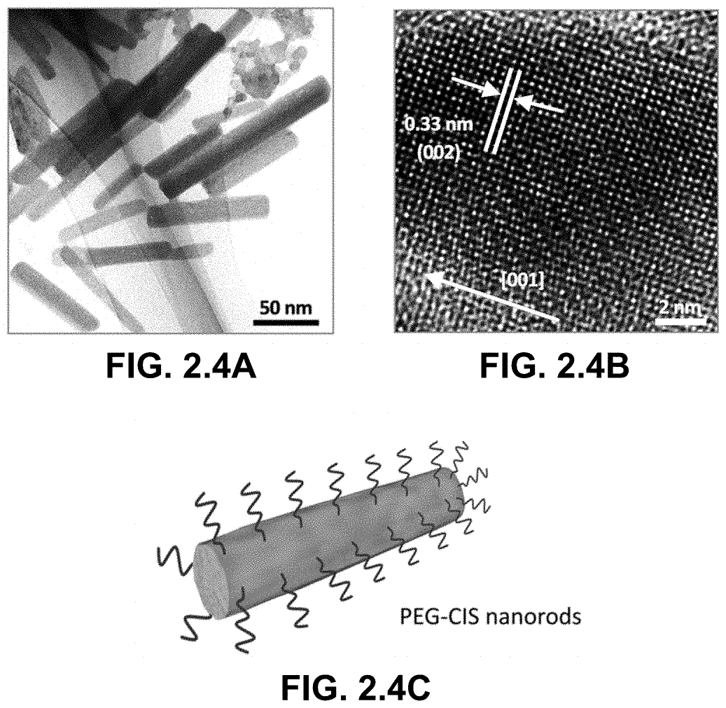

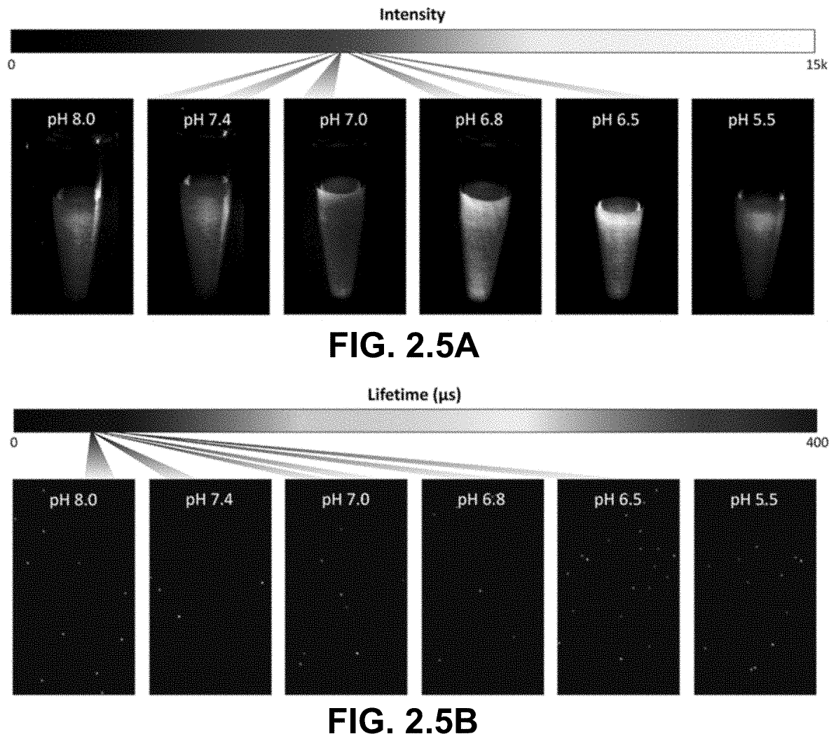

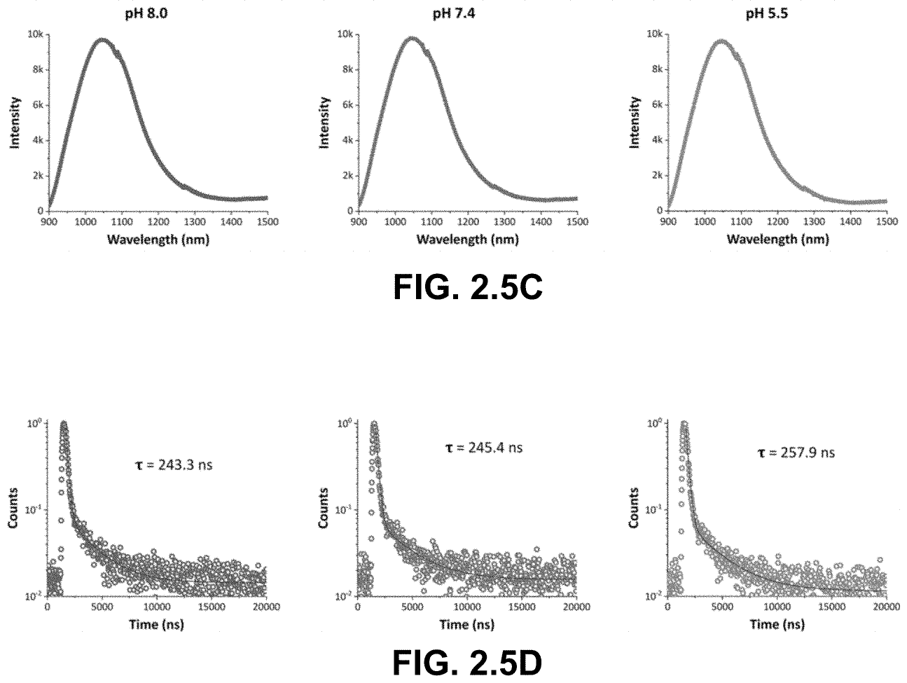

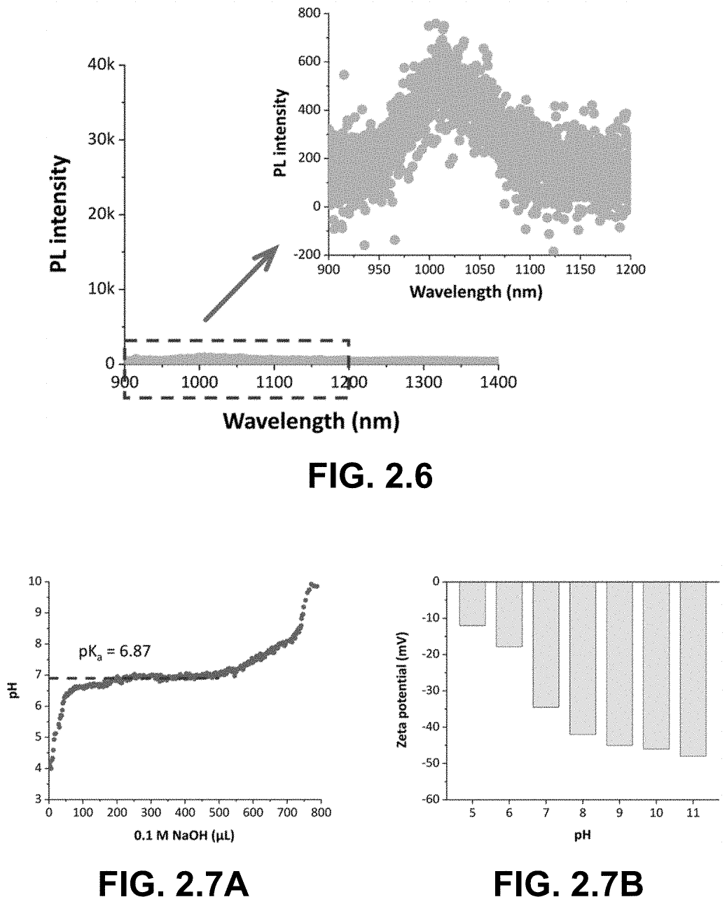

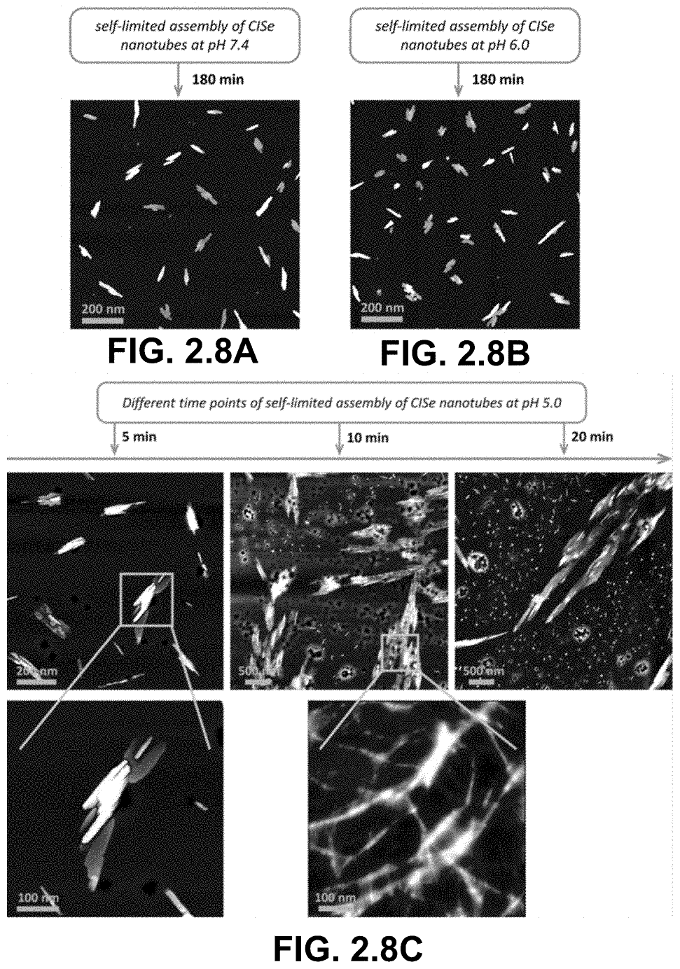

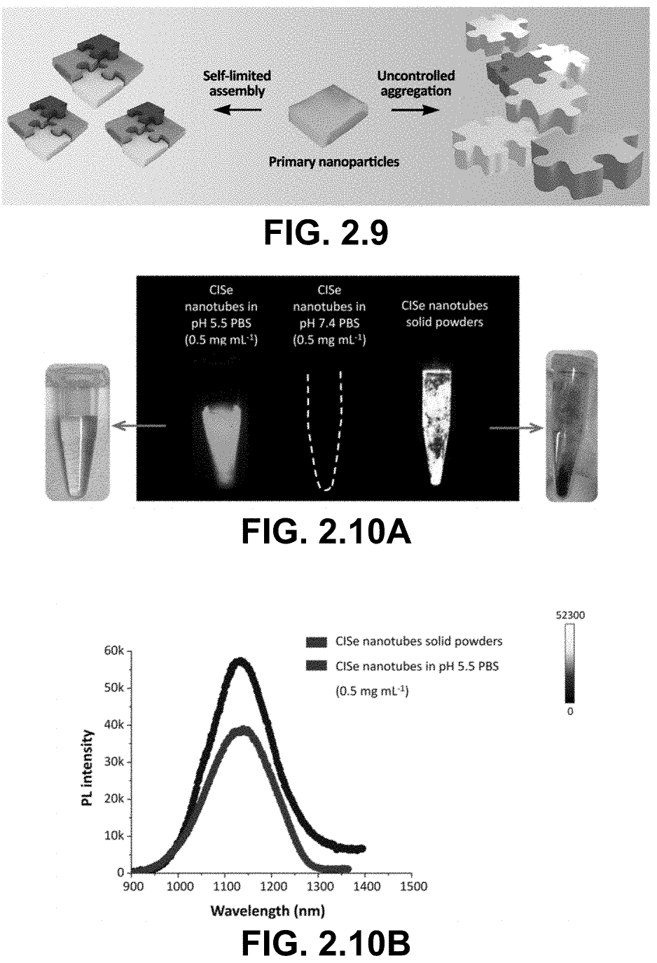

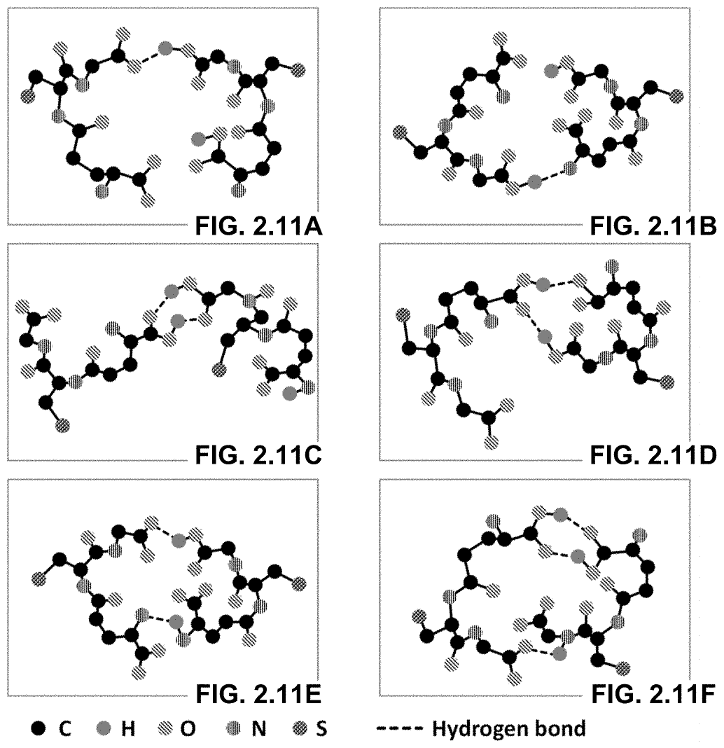

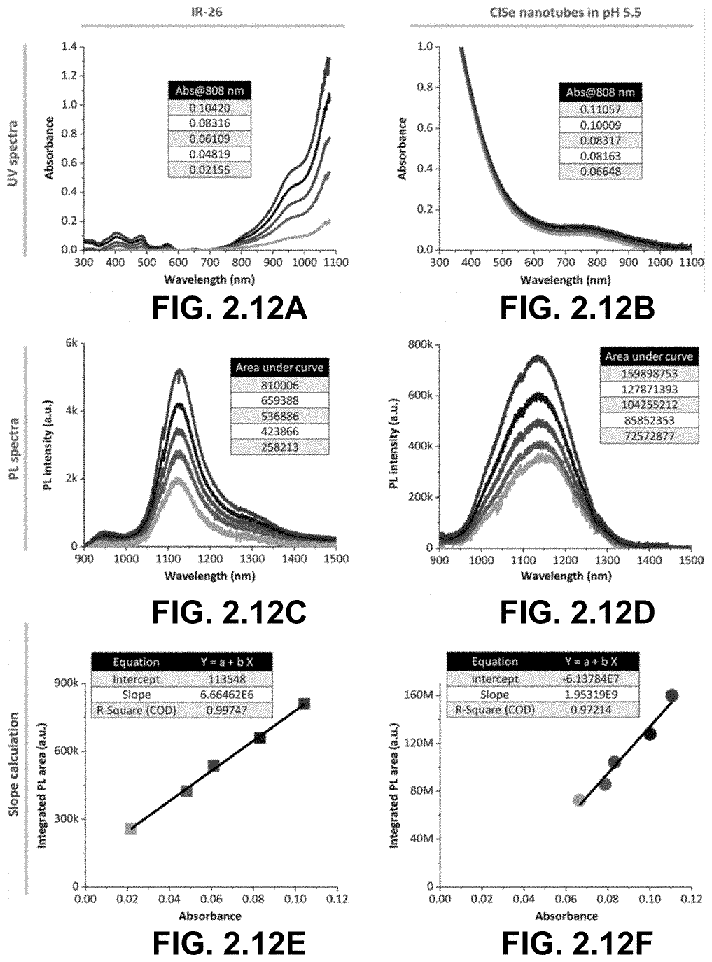

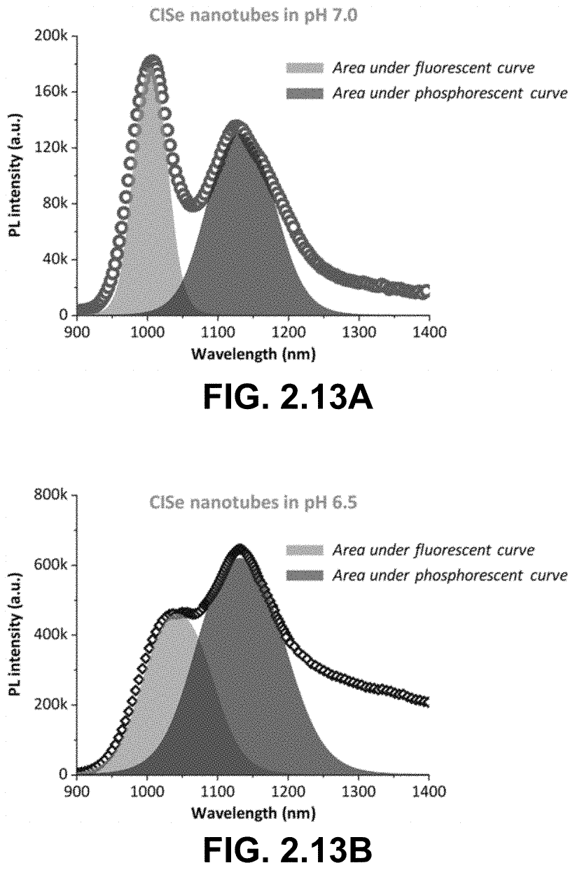

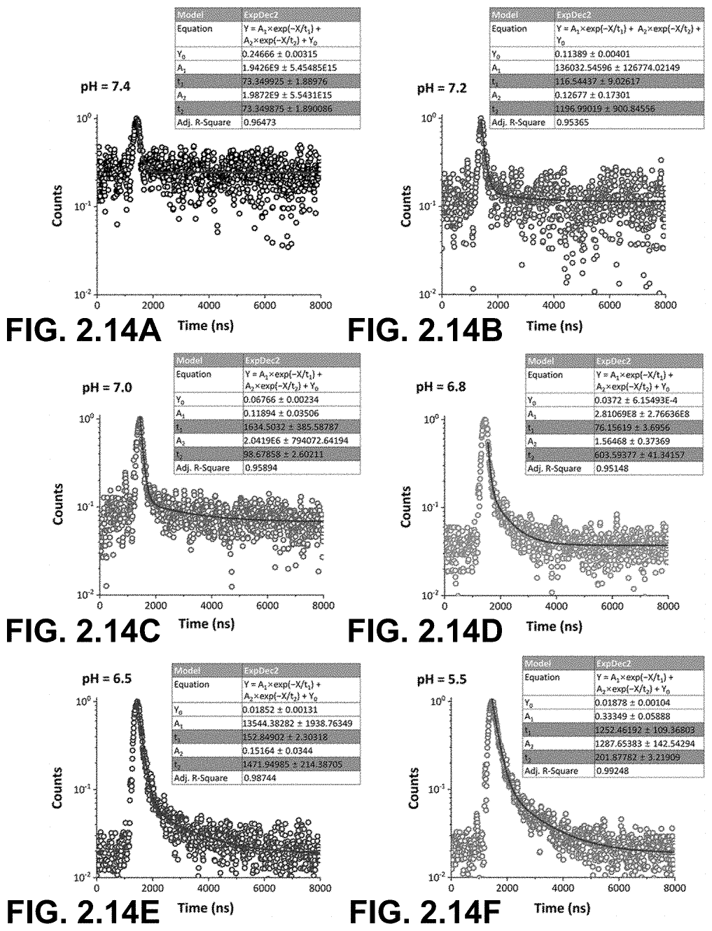

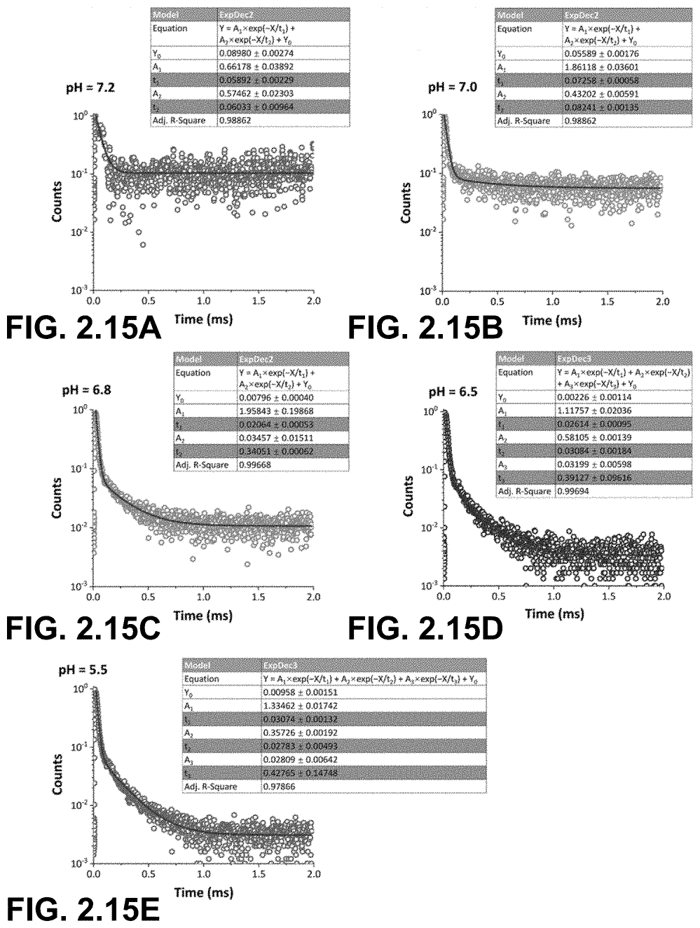

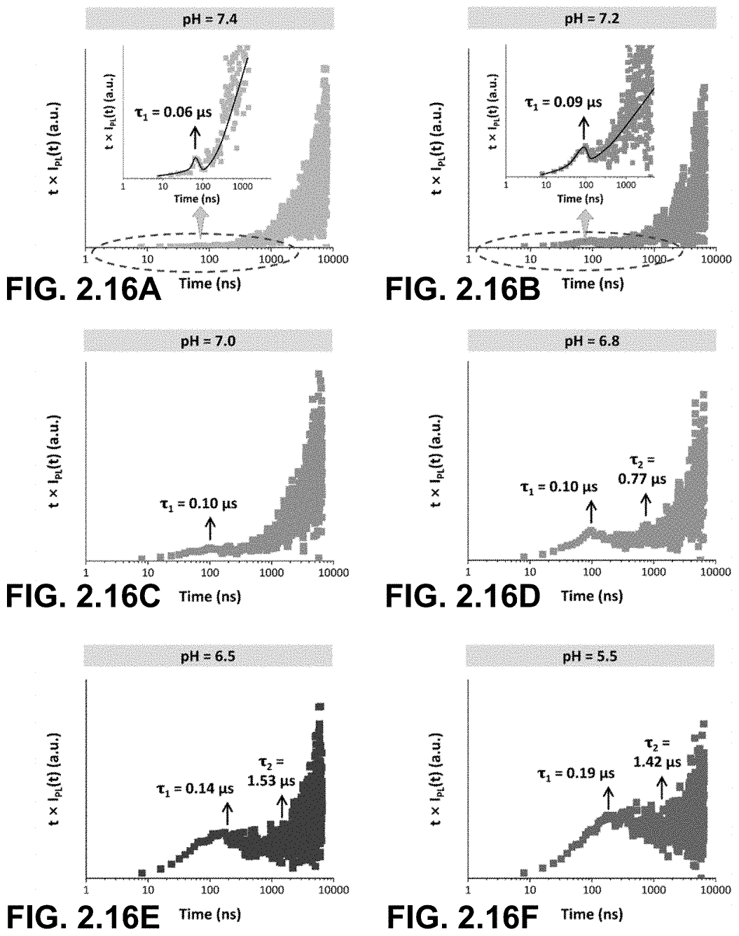

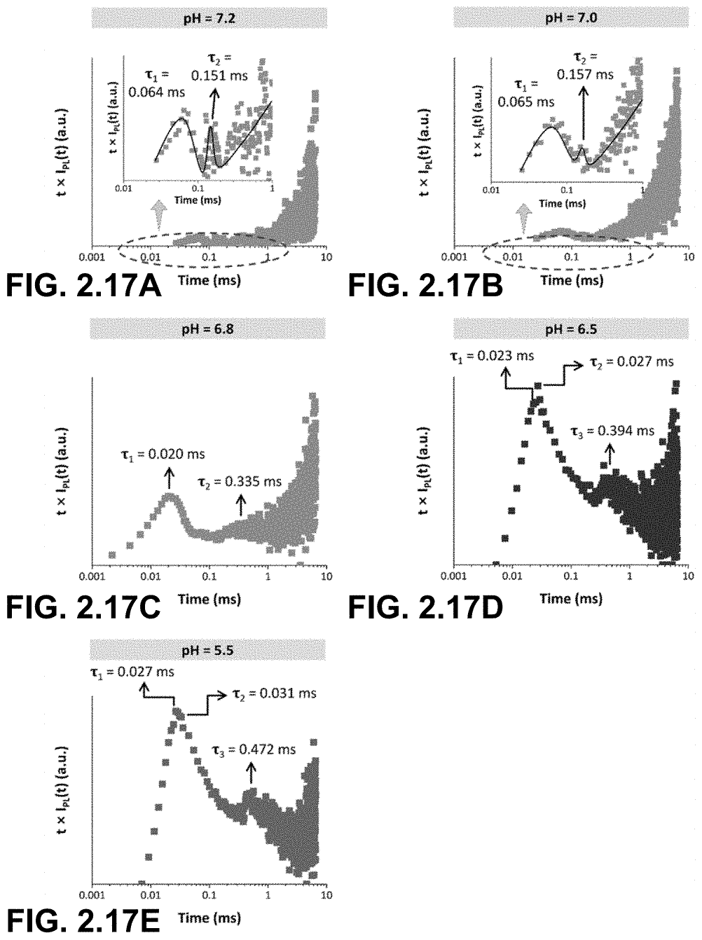

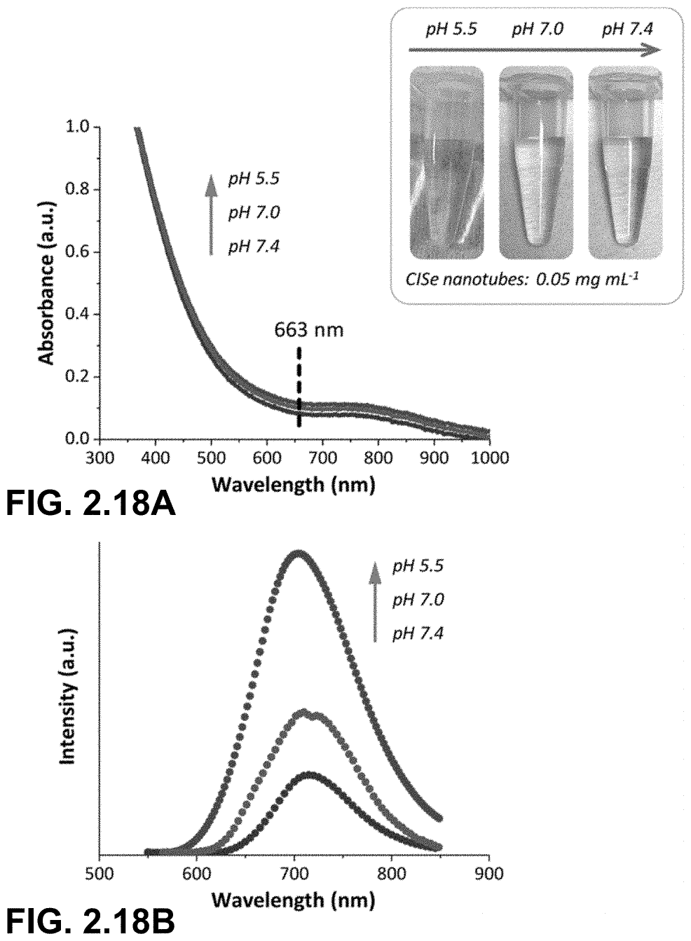

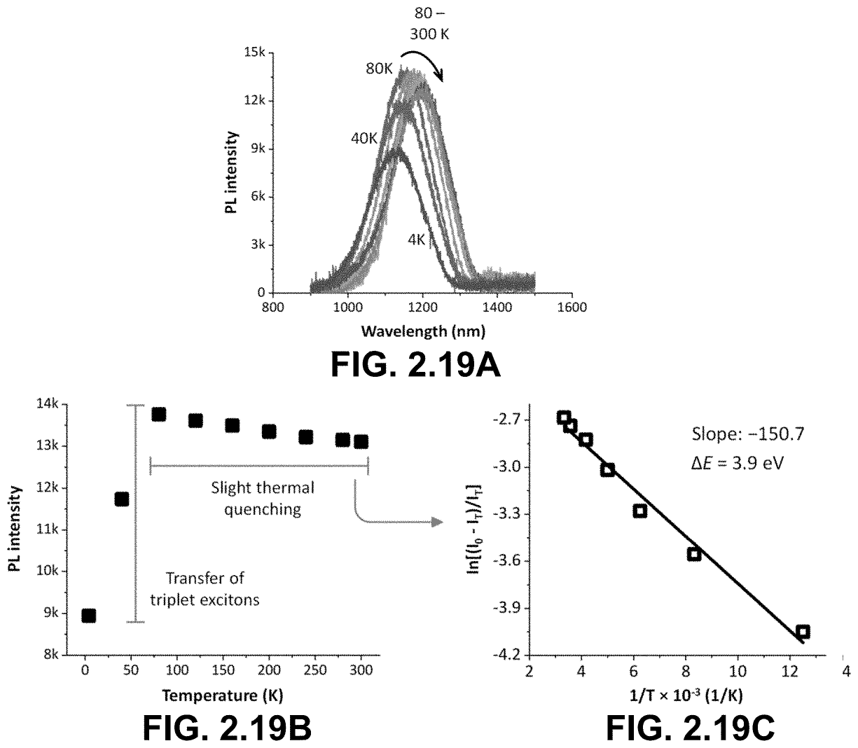

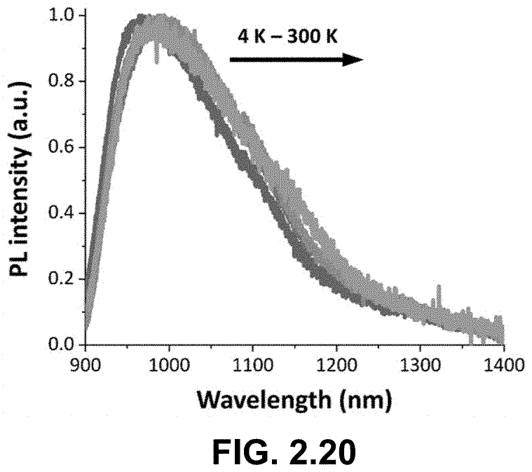

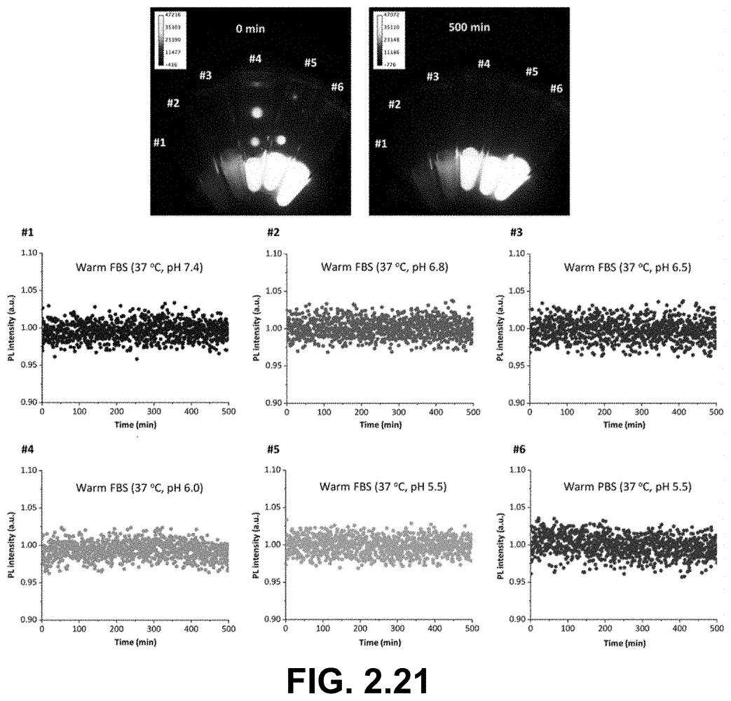

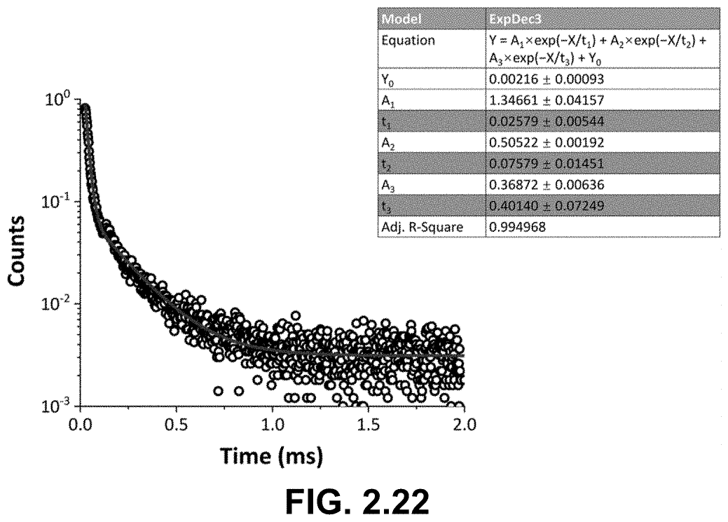

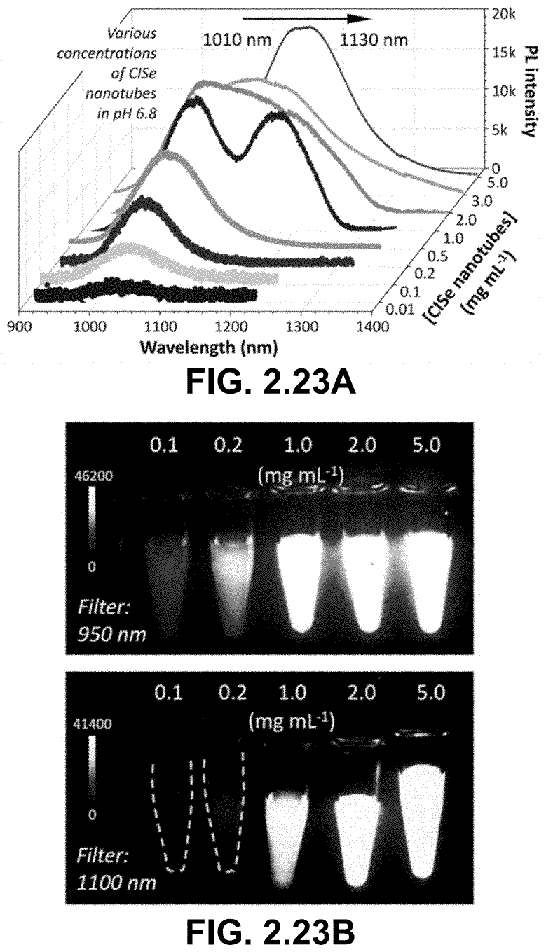

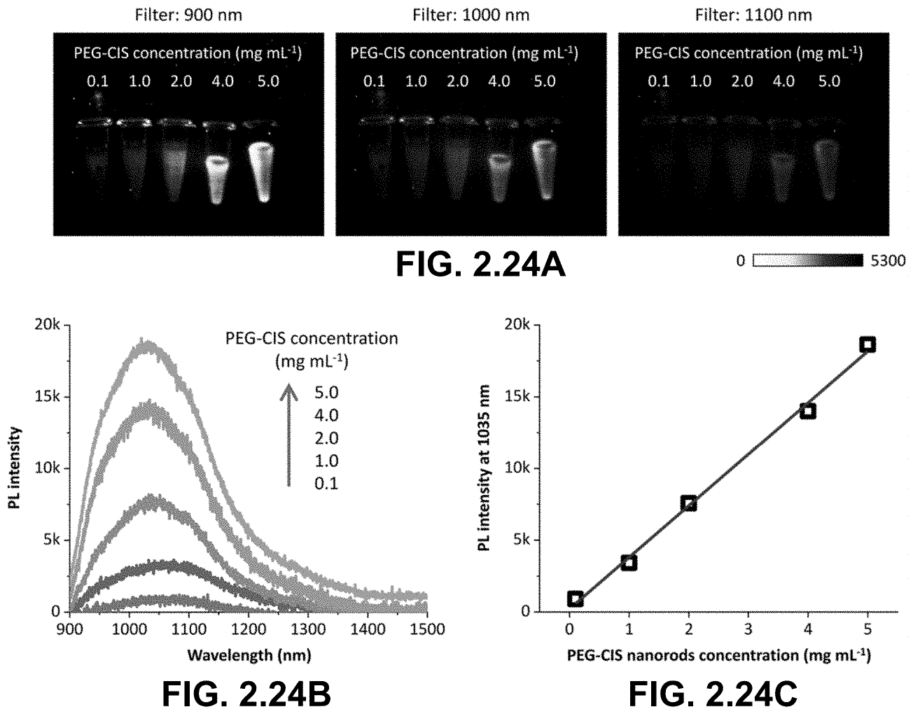

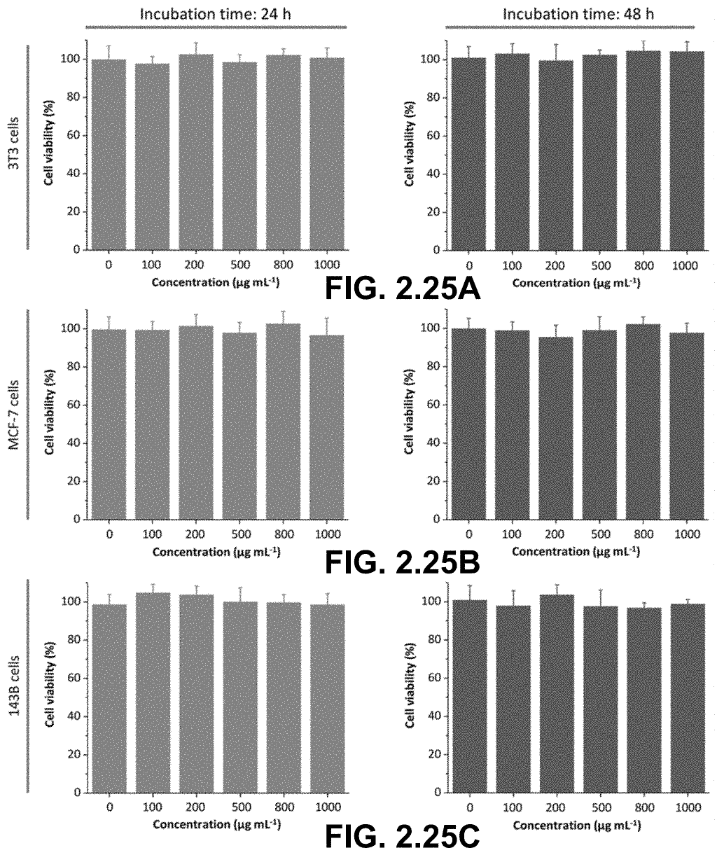

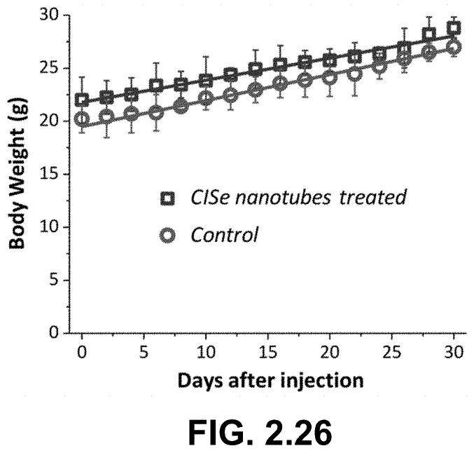

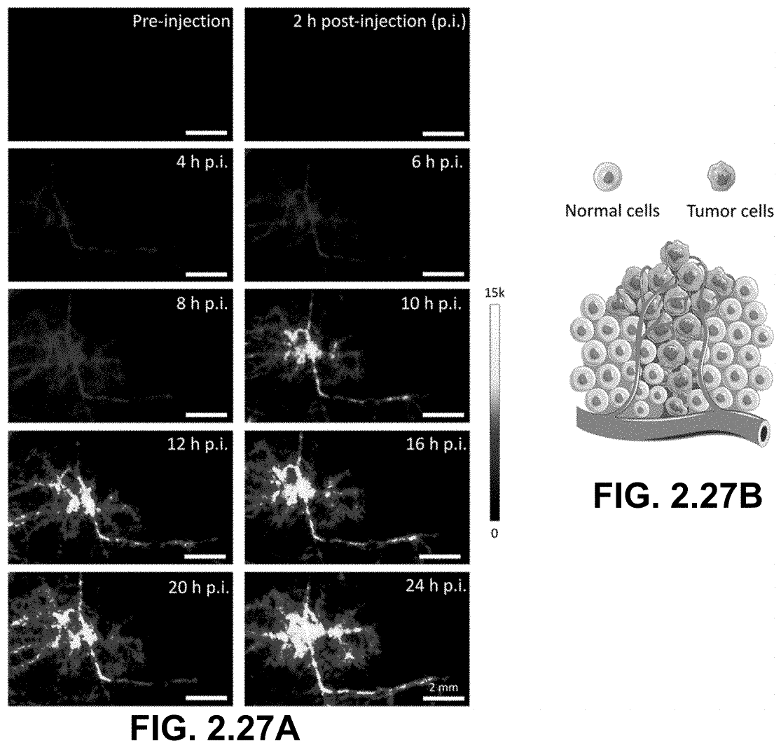

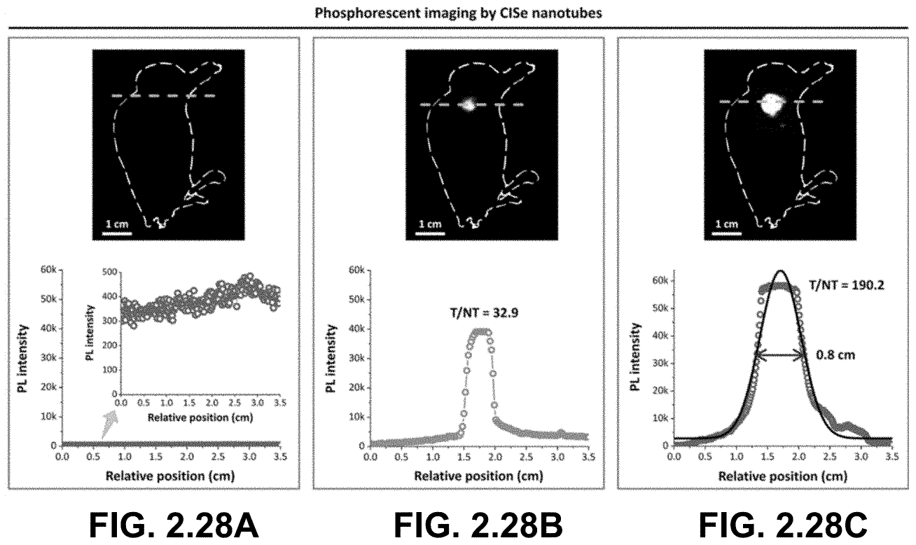

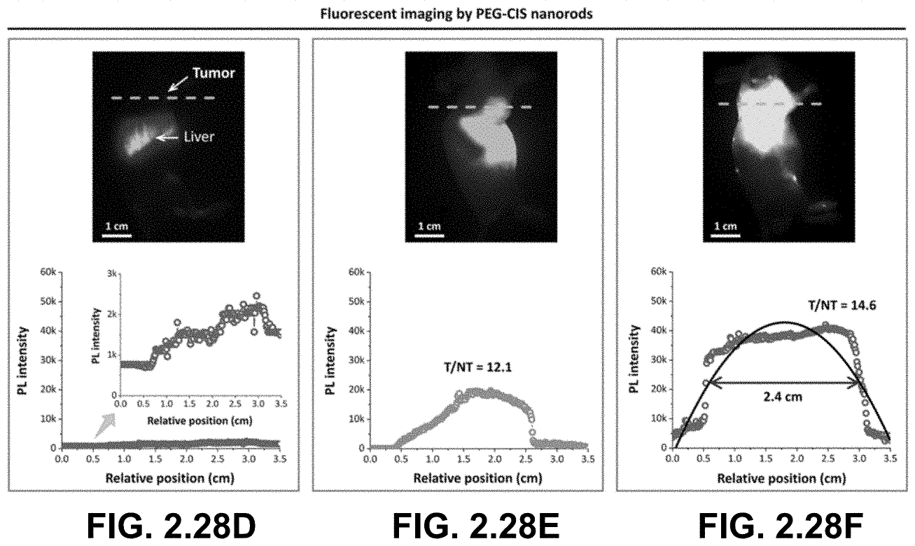

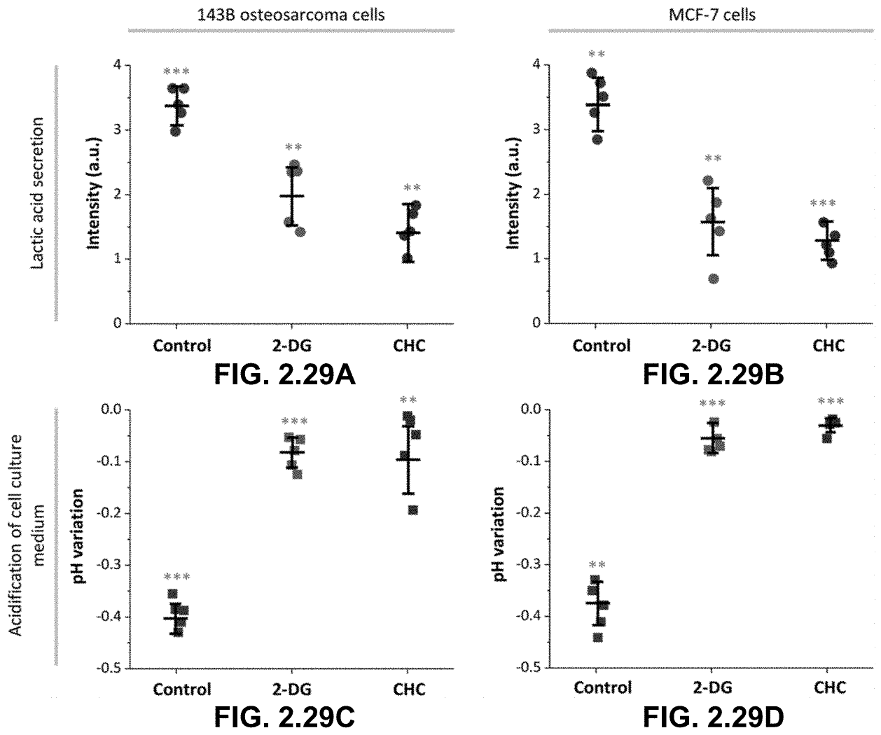

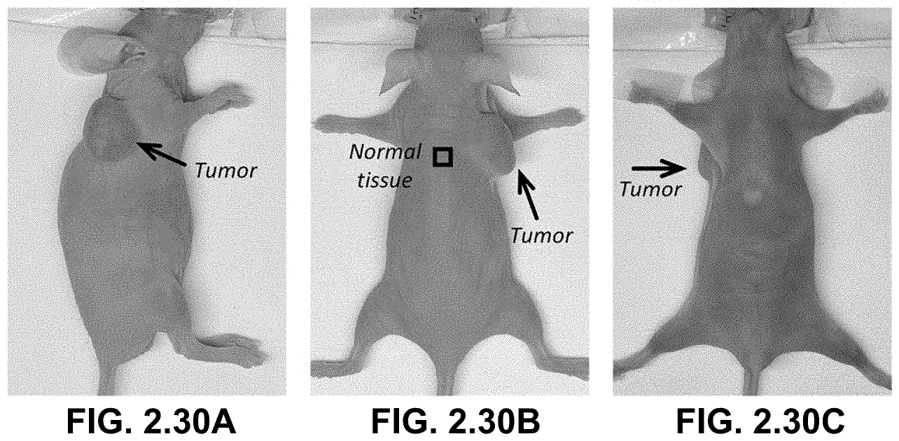

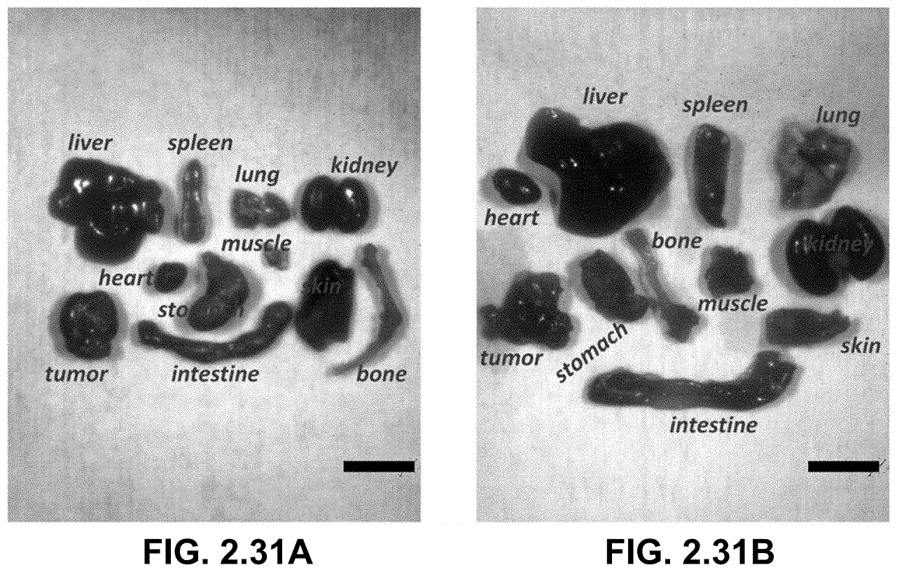

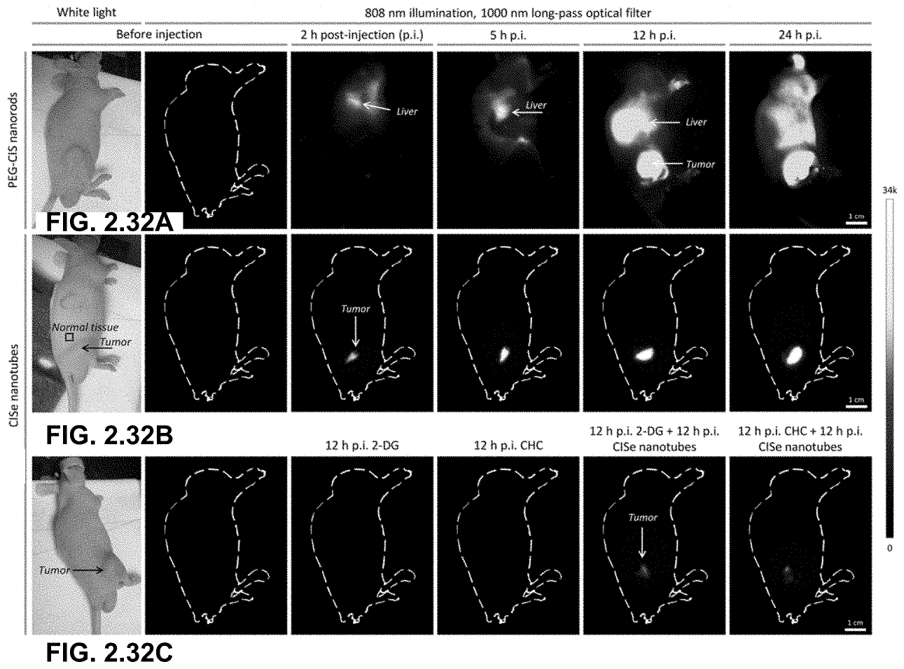

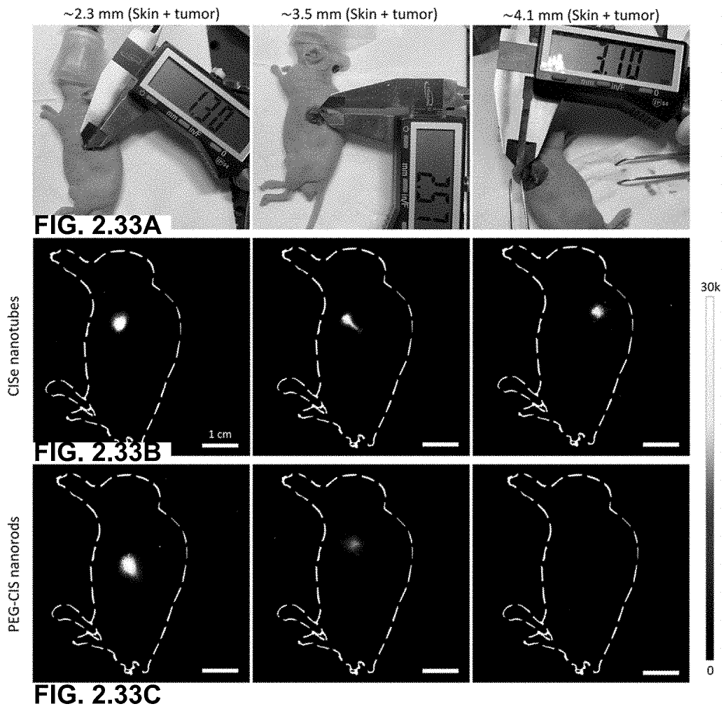

The synthesis of CISe nanotubes mainly involved two steps: preparation of Cu₂₋ₓSe nanorods as a template by water-evaporation-initiated self-assembly and partial cation exchange-induced hollow nanostructures¹³. Solid Cu₂₋ₓSe nanorods (average 1.8:1 Cu:Se atom ratio) were obtained with a mean diameter of 11.5 nm and length up to hundreds of nanometers (FIG. 2.1 from Example 2 below). By reacting Cu_{1.8}Se nanorods with InCl₃ for 1.5 h (the optimized reaction time), on average, 1 In³⁺ cation incorporated into the crystal lattice by replacing 2.9 host Cu⁺ cations (FIGS. 2.2, 2.3, and Table 1 from Example 2, below). As a result, the outgoing Cu⁺ cations diffused much more quantities than ingoing In³⁺ cations, which generated solely one-dimensional hollow space with a diameter of 3.8 nm as evidenced via Transmission electron microscopy (TEM) image and high-resolution TEM (HR-TEM) images in FIGS. 1.1B-1.1D. The diameter and length of CISe nanotubes were about 11.9 nm and 170 nm, respectively. Interestingly, shell of CISe nanotubes was shown to be composed of nine layers of highly ordered yet various lattice arrays. The fringes that were taken from interior domains of shell showed lattice spacings of 0.34 and 0.48 nm (FIG. 1.1D), indexing to the (100) and (004) planes of In₂Se₃ nanoparticles. The fringe of 0.34 nm, belonging to the (112) interplanar spacing of chalcopyrite CuInSe₂ nanocrystals was identified throughout the whole shell of CISe nanotubes. It became evident that the alloyed compositions, i.e. In-rich inner shell, existed in CISe nanotubes. To further clarify the heterogeneous nature, high-angle annular dark-field scanning TEM (HAADF-STEM) was used and the mapping of element distributions in FIG. 1.1E suggested the presence of thick In-rich inner green domains highlighted by a contrast difference against red Cu domains. Analysis of mean compositions identified a slight indium excess with an average Cu:In:Se atomic ratio of 0.72:1.01:2 (Table 1, in Example 2, below).

Photophysical Properties of CISe Nanotubes

Photophysical studies were then performed on CISe nanotubes in phosphate buffered saline (PBS) with a broad range of pH values. 1,2-distearoyl-sn-glycero-3-phosphoethanolamine-N-[methoxy(poly-ethylene glycol)-2000]-stabilized copper indium sulfide (denoted as PEG-CIS) nanorods were utilized as a control nanoprobe with comparable morphology, size and chemical composition to the CISe nanotubes (FIG. 2.4). The relative luminescent brightness was monitored by imaging the tubes on InGaAs camera (FIG. 1.2A). Surprisingly, CISe nanotubes showed negligible optical signals in PBS from pH 8.0 to 7.0 (FIG. 1.2B). Upon changing pH to 6.8, this narrow pH difference gave an intense NIR-II light, suggesting a sharpness of PL activation. The lower pH favored the stronger emission intensity. In contrast, PL intensities of PEG-CIS nanorods maintained constant irrespective of pH values (FIG. 2.5A, Example 2, below).

To understand PL activation, PL spectra of CISe nanotubes in various pH were measured (FIG. 1.2C). CISe nanotubes were PL-inactive in an alkaline pH range. By gradually lowering pH to 7.0, PL spectrum split into the bimodal pattern: a new NIR-II band at 1130 nm together with an original one at 1010 nm that could be vaguely seen in pH 7.4 (FIG. 2.6, Ex. 2). Taking a close look at PL spectra from pH 7.4 to 5.5, the peak intensities of 1010 nm and 1130 nm possessed totally opposite varying directions. The 1130 nm peak enormously intensified and reached a plateau below pH 6.0, showing about 5.8×10³-fold increase in intensity relative to pH 7.4 (FIG. 1.2D). Of note, another peak centering at 1010 nm decreased rapidly at pH below 6.5. The pKₐ of 6.87 estimated by acid-base titration (FIG. 2.7A, Ex. 2) was very close to the pH transition point that governed PL activation process (FIG. 1.2D). Moreover, the pH difference (ΔpH) regarding a range of 10-90% intensity was only 0.4, much sharper than that of most probes (˜2 pH units)¹⁵.

TEM and atomic force microscopy (AFM) were performed to record the colloidal stability of CISe nanotubes in various pH values. CISe nanotubes remained electrostatically stabilized (Zeta potential in FIG. 2.7, Ex. 2) and were dissolved in a highly isolated dispersible state at pH 7.4 (FIG. 1.2E and AFM images in FIG. 2.8 from Ex. 2). As the pH reduced to 6.0, CISe nanotubes readily aggregated into stable compact clusters without a tendency to form large aggregate and precipitate. It was indicative of self-limited assembly event (Ex. 2, FIG. 2.9), conceptually similar to supraparticles reported before Upon further varying pH to 5.0, this self-limited assembly behavior quickly deteriorated to some extent yet still remained compact arrangement of CISe nanotubes (FIG. 1.2E and Ex. 2, FIG. 2.8C). The union of above pH-driven PL activation and colloidal stability inferred that the compact arrangement can efficiently improve the emission intensity of CISe nanotubes, showing an assembly-dependent PL enhancement. Intriguingly, this phenomenon was highly amplified in solid states (FIG. 1.2B and Ex. 2, FIG. 2.10). The hand-drawn “bulb” stick figure using CISe nanotubes powders as a “ink” was vividly monitored.

As illustrated in FIG. 1.2F, self-limited assembly of CISe nanotubes can be explicitly rationalized by a delicate balance between electrostatic repulsive force and multiple interparticle H-bonding attractive force (Ex. 2, FIG. 2.11). The clusters' growth will cease as soon as the repulsive force between clusters and primary nanotube equaled the attractive force. Declining pH further intensified this assembly extent and yielded denser products. The compact arrangement, greatly restricting the vibration and rotation of ligands, enhanced radiative relaxation of excited states and resulted in a profound increase of PL intensity¹⁷.

The properties of two PL bands centering at 1010 and 1130 nm that seemed to be mutually exclusive encouraged a look into the photophysical origin of CISe nanotubes. In FIG. 1.21, time-resolved PL decay measurements analyzed by nonlinear least-squares fitting method (see Ex. 2, FIGS. 2.14 and 2.15 for detailed calculation process of lifetimes) indicated the predominance of nanosecond lifetime components for the emission peak at 1010 nm, which double-exponentially decayed regardless of pH values (Table 2 in Ex. 2, below). In contrast, the peak centering at 1130 nm displayed an interesting pH-dependent decay nature from double-exponential at approximately neutral conditions (pH 7.2, 6.8) to triple-exponential at an acidic pH scope of 6.5-5.5 (FIG. 1.2J and Ex. 2, FIGS. 2.15, 2.17). More importantly, these multi-exponential decays typically occurred on a time scale of hundreds of microseconds. At pH 5.5, the lifetime significantly increased to 336.1 μs, which was approximately 4.6×10³-fold of the original one at pH 7.4 with a sharper ΔpH of 0.2 (FIGS. 1.2K and 1.2L). Such unique long-lived properties were verified by time-resolved NIR-II imaging system (FIG. 1.2G). Reducing pH values from 8.0 to 5.5 significantly increased the lifetimes of CISe nanotubes (FIG. 1.2H). For example, lifetime imaging at pH 6.5 facilely recognized the tubes against background with consistent pseudo-colors, hinting high reliability of long-lived signals. However, lifetime signal was hardly detectable in PEG-CIS nanorods, owing to their much shorter lifetimes (Ex. 2, FIGS. 2.5B and 2.5D). Time-resolved NIR-II imaging results confirmed that the long-lived NIR-II emission with large Stokes shift and high quantum yield (Table 3, below) was very helpful to minimize background interferences with nanoseconds-scaled lifetimes.

Transient absorption (TA) spectroscopy and low-temperature PL spectroscopy were then used to clarify excited-state relaxation behind above long-lived NIR-II signals. Compared with spectral results that had markedly weak TA responses in pH 7.4 (FIGS. 1.3A and 1.3B), photoexcited CISe nanotubes in pH 6.0 distinctly featured a ground-state bleach (GSB) band at 663 nm (FIGS. 1.3D and 1.3E), ascribing to S₁ absorption edge (Ex. 2, FIG. 2.18). This singlet excited state rapidly declined in 38.4 μs accompanying by emergence of a second feature of excited state absorption (ESA) at 462 nm, which cannot be optically excited and thus was assigned to a dark state absorption from Cu-rich region (FIG. 1.3G and Ex. 2, FIG. 2.18)¹⁸. Notably, this ESA decay kinetics coincided with the presence of a third feature of ESA at 957 nm, displaying a longer lifetime as indicated by staying constant within measurement time window (FIG. 1.3F). Accordingly, the 957 nm ESA was assigned to triplet absorption in In-rich domain (FIG. 1.3G) from the lowest state (T₁) to a higher lying triplet state (Tₙ). By elevating temperatures from 4 to 80 K (FIG. 1.3H and Ex. 2, FIG. 2.19), over 1.5-fold intensity increasement confirmed the exciton transfer dynamics that photoexcitation-induced singlet excitons could rapidly transfer to low-lying In-rich states at higher temperature, a similar process to internal conversion by thermal vibrational relaxation¹². The large activation energy (ΔE, 3.9 eV), corresponding to nonradiative decay pathway, resulted in slight thermal quenching, which enabled excellent photostability of CISe nanotubes in diverse environments (FIGS. 1.2M and 1.2N and Ex. 2, FIG. 2.21). Besides, CISe nanotubes presented a good PL reversibility toward a pH cycling between 6.0 and 7.4 (FIG. 1.2m).

FIG. 1.31 illustrated excited-state relaxations and highlighted that Cu-to-In exciton transfer was identified as the origin for the generation of phosphorescence. After the photoexcitation singlet excitons were created within picosecond time scale, they will decay back to Cu-rich state in 48.6 μs and subsequently converted to triplet excitons in the In-rich state with 28.9 μs time constant. The self-limited assembly of CISe nanotubes can stabilize and facilitate Cu-to-In exciton transfer (Ex. 2, FIGS. 2.22-2.24), which then decayed to the ground state by emitting a strong long-lived phosphorescence.

Ultrahigh Tumor-Specific Imaging

The intriguing PL properties of CISe nanotubes inspired validation of their application for in vivo tumor imaging, which still suffers from poor sensing precision to date. Prior to bioimaging, the biosafety of CISe nanotubes was a big concern. In vitro cell viability assays revealed minimal cytotoxicity associated with CISe nanotubes (Ex. 2, FIG. 2.25). To investigate the in vivo toxicity, normal mice were treated with CISe nanotubes at a dosage of 100 mg kg⁻¹. There were no significant differences in the body weights over 30-day period between treatment group and the control (Ex. 2, FIG. 2.26). Also, no side effects on physical signs and behaviors were found. For the blood biochemistry test, negligible fluctuation of 12 important hepatic, heart and kidney function markers indicated no acute toxicity induced (FIGS. 1.4A, 1.4B, 1.4C). The H&E stained images (FIG. 1.4D) indicated that structural patterns of major organs harvested on day 30 from mice in the experimental group were similar to control group without signs of damage and other symptoms. These data collectively indicated good biocompatibility of CISe nanotubes.

For tumor bioimaging, CISe nanotubes were intravenously injected at a dosage of 20 mg kg⁻¹ into nude mice bearing subcutaneous 143B osteosarcoma cancer cells. The intensity-based and time-resolved imaging systems were used to collect serial whole-body NIR-II images at different time points, respectively (FIGS. 1.5A and 1.5B). Very fascinatingly, CISe nanotubes yielded heterogeneous imaging pattern via time-resolved imaging, indicative of the presence of acidity-rich area, which cannot be observed crossing the whole tumor site under intensity-based NIR-II imaging (FIG. 1.5C). Notably, at early post-injection time of 2 h, an average lifetime of 78 μs (FIG. 1.51) was obtained in tumor as evident by a contrast difference against the background. However, by switching to the intensity imaging mode, almost no intensity signal was observed, inferring that long-lived NIR-II phosphorescence markedly differentiated autofluorescence in time domain with higher sensing sensitivity. At the late post-injection time, PL intensity signals in tumor became evidently visible as background maintained clean over time and more so after 24 h. Along with the through-skin, in vivo real-time imaging of tumor vessels (Ex. 2, FIG. 2.27), a main vessel with diameter of ˜7.6 μm could be facilely resolved with a signal-to-noise ratio of ˜16.3 (FIG. 1.5J), revealing a good spatial resolution of NIR-II phosphorescence imaging.

As a control NIR-II fluorescent nanoprobe, however, the PEG-CIS nanorods became invisible by time-resolved imaging mode, due to their fast decaying fluorescence with a short lifetime of below 260 ns that will be filtered by time-gated imaging systems (FIG. 1.5D and Ex. 2, FIG. 2.5). Under intensity-based NIR-II imaging systems, PEG-CIS nanorods gave intensity signals inside tumor location and other normal tissues (e.g., liver and intestine), leading to strong background interferences (FIG. 1.5D). Thereby, compared with gradually faded signal ratios of tumor-to-normal tissue (T/NT) associated with PEG-CIS nanorods over time, T/NT values of CISe nanotubes-injected mice markedly reached up to 190±10 at 24 h (FIG. 1.5K). On average, an impressive 38-fold higher level of signal-to-noise ratio than the Rose criterion was verified, which suggests that the T/NT value of 5 is prerequisite for identifying tumor image feature with 100% certainty¹. The cross-sectional PL intensity profiles analysis (Ex. 2, FIG. 2.28) hinted that CISe nanotubes achieved high-precision tumor imaging compared with PEG-CIS nanorods. These ultrahigh tumor imaging data validated that subtle, physiologically relevant pH trigger was sufficient to activate long-lived phosphorescence of CISe nanotubes.

To study the pH specificity of CISe nanotubes, two inhibitors for tumor glycolysis were used to demonstrate proof of principle of acidic extracellular pHₑ-specific tumor imaging. In vitro cell culture studies showed statistically significant inhibition of lactic acid secretion after incubation by 2-deoxy-D-glucose (2-DG) or α-cyano-4-hydroxycinnamate (CHC), thus retarding acidification of cell culture medium (FIG. 1.5F and Ex. 2, FIG. 2.29), respectively. Pretreating mouse with either metabolic inhibitor caused substantially quenching phosphorescent signals in terms of PL intensity and lifetime (FIG. 1.5E). Nonetheless, both inhibitors were ineffective for the mouse treated with pH-inert PEG-CIS nanorods. These interesting results, made on the basis of blocking glycolysis pathways, convincingly demonstrated that CISe nanotubes could specifically target the acidic tumor extracellular pHₑ.

After 24 h post-injection of nanoprobes, the mice were euthanized, and excised organs were imaged under intensity-based mode (FIGS. 1.5G and 1.5H). CISe nanotubes only exhibited strong brightness in tumor, whereas PEG-CIS nanorods generated widespread intensity signals in tumor and other non-tumor tissues. The quantified PL intensities inferred that the tumor-to-liver ratio, a critical parameter for tumor imaging, markedly increased to 110±40 for CISe nanotubes (FIG. 1.5L), over 400-fold higher than the average value of 0.28 in previously reported inorganic probe systems²⁰. Moreover, CISe nanotubes simultaneously exhibited high signal ratios of tumor-to-major organs outperforming those from PEG-CIS nanorods, in line with data under whole-body NIR-II imaging. Of note, the developed NIR-II phosphorescent imaging, which seemed to be a broadly applicable strategy (Ex. 2, FIG. 2.32), was preferable for in vivo tumor-specific imaging with good sensitivity and precision.

Encouraged by above results, the benefit of NIR-II phosphorescent imaging was explored both in vivo and in vitro. The tissue phantom study with 1% Intralipid that mimics the optical properties of biological tissues was performed to compare the clarity and penetration depths of CISe nanotubes and PEG-CIS nanorods (FIG. 1.6A). The PL signal intensities linearly correlated with the probe concentrations (FIGS. 1.6B-1.6D). Taking 2 mg mL⁻¹ of probes as an example, attenuation of image intensities and blurring of capillary profiles can be found for both probes with increase of penetration depths (FIG. 1.6E). However, the full-width at half-maximum (FWHM) measurements identified that CISe nanotubes still resolved sharp edge of capillary at depths up to 5 mm with a signal-to-noise ratio of ˜15.6, while PEG-CIS nanorods at 5 mm only had signal-to-noise ratio of ˜1.9 (FIGS. 1.6F and 1.6G). Phantom studies verified better spatial resolutions of NIR-II phosphorescence at deeper tissue penetration over NIR-II fluorescence, which can be further established by imaging tumor-bearing nude mice injected with nanoprobes intratumorally at various sites (FIGS. 1.6H and 1.61 and Ex. 2, FIG. 2.33). Quantitative analysis of cross-sectional intensity profiles validated that both the imaging brightness and signal-to-noise ratios in tumors treated with PEG-CIS nanorods markedly dropped compared with that of CISe nanotubes for all imaging depths (FIGS. 1.6J and 1.6K).

### DISCUSSION

In vivo tumor imaging by optical probes still faces many challenges unless they concurrently address issues including, but not limited to, the following aspects. (1) Limited spatial resolution and tissue penetration depth. The majority of probes, emitting in a visible (400-650 nm) and the first NIR window (650-950 nm), realize tissue penetration depths of only several millimeters^{21,22}. (2) Suboptimal emission mode. In contrast to probes with fluorescence, phosphorescence holds vast potential for in vivo imaging, since its much longer lifetime (μs to s scale) will enable time-delayed imaging to prevent autofluorescence with ns-scale lifetime²³. (3) Spectral crosstalk. The small Stokes shift is insufficient to separate excitation and emission wavelengths, hence causing remarkable self-quenching (4) Unspecific imaging pattern. In vivo imaging by common probes that continuously yield PL regardless of whether they reach pathological sites suffers from poor sensing precision²⁵. Mainly owing to currently inadequate design concepts, unfortunately, NIR-II phosphorescent probe is still in its infancy.

The present example describes a physiological pH-induced radiative mode switch strategy to employ NIR-II phosphorescent imaging, which can address above-mentioned photophysical, biological and in vivo tumor imaging barriers. Cu-to-In exciton transfer stabilized by self-limited assembly was identified as the origin of the generation of long-lived NIR-II phosphorescence. NIR-II phosphorescent imaging brings more opportunities to address important unmet biomedical needs, for example in vivo tumor-specific imaging. Notably, acidic tumor microenvironment deriving from dysregulated glycolysis, termed “Warburg effect”, has been recognized as one hallmark of tumors²⁶, regardless of genotypes and phenotypes. Thus, imaging a tumor via targeting its acidic pH, (6.5-6.8) is can be empoyed as a universal method in broad tumor detection^{27,28}. The difficulty thus far has been how to rapidly and specifically distinguish small pH differences between acidic tumor pH, and blood (pH 7.4)¹⁵. Under time-resolved imaging systems, the sharp and exquisite pH sensitivity of long-lived NIR-II phosphorescence from CISe nanotubes may pave the way to circumvent tumor heterogeneity for in vivo imaging.

### CONCLUSION

Taken together, the functional integration of benefits from NIR-II phosphorescence and NIR-II imaging modality demonstrate the ability to achieve ultrahigh tumor-specific imaging with large spatial resolutions and deep tissue penetration depths. The developed NIR-II phosphorescent imaging, working by radiative mode switch and long emission lifetime, will afford extensive opportunities to address different important unresolved obstacles across early diagnosis, timely intervention and clinical translation of nanomedicine.

### References for Example 1

1. Antaris, A. L., et al. A small-molecule dye for NIR-II imaging. Nat. Mater. 15, 235-242 (2016).
2. Hu, Z. H., et al. First-in-human liver-tumour surgery guided by multispectral fluorescence imaging in the visible and near-infrared-I/II windows. Nat. Biomed. Eng. 4, 259-271 (2020).
3. Chen, X. H., Chen, Y. X., Xin, H. H., Wan, T. & Ping, Y. Near-infrared optogenetic engineering of photothermal nanoCRISPR for programmable genome editing. P. Natl. Acad. Sci. USA 117, 2395-2405 (2020).
4. Wang, W., et al. Metabolic labeling of peptidoglycan with NIR-II dye enables in vivo imaging of gut microbiota. Angew. Chem. Int. Ed. 59, 2628-2633 (2020).
5. Fan, Y., et al. Lifetime-engineered NIR-II nanoparticles unlock multiplexed in vivo imaging. Nat. Nanotechnol. 13, 941-946 (2018).
6. Zhang, M., et al. Bright quantum dots emitting at −1,600 nm in the NIR-Ilb window for deep tissue fluorescence imaging. P. Natl. Acad. Sci. USA 115, 6590-6595 (2018).
7. Lei, Z., et al. Stable, wavelength-tunable fluorescent dyes in the NIR-II region for in vivo high-contrast bioimaging and multiplexed biosensing. Angew. Chem. Int. Ed. 58, 8166-8171 (2019).
8. Shapiro, A., et al. Tuning optical activity of IV-VI colloidal quantum dots in the short-wave infrared (SWIR) spectral regime. Chem. Mater. 28, 6409-6416 (2016).
9. McHugh, K. J., et al. Biocompatible semiconductor quantum dots as cancer imaging agents. Adv. Mater. 30, 1706356 (2018).
10. Zhu, S. J. & Chen, X. Y. Overcoming the colour barrier. Nat. Photonics 13, 515-516 (2019).
11. Su, Y., et al. Ultralong room temperature phosphorescence from amorphous organic materials toward confidential information encryption and decryption. Sci. Adv. 4, eaas9732 (2018).
12. Zhao, W. J., et al. Boosting the efficiency of organic persistent room-temperature phosphorescence by intramolecular triplet-triplet energy transfer. Nat. Commun. 10, 1595 (2019).
13. Xu, J., et al. Large-scale synthesis of long crystalline Cu₂₋ₓSe nanowire bundles by water-evaporation-induced self assembly and their application in gas sensing. Adv. Funct. Mater. 19, 1759-1766 (2009).
14. Xu, J., et al. Large-scale synthesis and phase transformation of CuSe, CuInSe₂, and CuInSe₂/CuInS₂ core/shell nanowire bundles. ACS Nano 4, 1845-1850 (2010).
15. Urano, Y., et al. Selective molecular imaging of viable cancer cells with pH-activatable fluorescence probes. Nat. Med. 15, 104-109 (2009).
16. Xia, Y. S., et al. Self-assembly of self-limiting monodisperse supraparticles from polydisperse nanoparticles. Nat. Nanotechnol. 6, 580-587 (2011).
17. Mou, M., et al. Aggregation-induced emission properties of hydrothermally synthesized Cu—In—S quantum dots. Chem. Commun. 53, 3357-3360 (2017).
18. Jara, D. H., Stamplecoskie, K. G. & Kamat, P. V. Two distinct transitions in CuxInS₂ quantum dots. Bandgap versus sub-bandgap excitations in copper-deficient structures. J. Phys. Chem. Lett. 7, 1452-1459 (2016).
19. Liu, X., Braun, G. B., Qin, M., Ruoslahti, E. & Sugahara, K. N. In vivo cation exchange in quantum dots for tumor-specific imaging. Nat. Commun. 8, 343 (2017).
20. Yu, M. X. & Zheng, J. Clearance pathways and tumor targeting of imaging nanoparticles. ACS Nano 9, 6655-6674 (2015).
21. Zhang, R. R., et al. Beyond the margins: Real-time detection of cancer using targeted fluorophores. Nat. Rev. Clin. Oncol. 14, 347-364 (2017).
22. Li, J. & Pu, K. Semiconducting polymer nanomaterials as near-infrared photoactivatable protherapeutics for cancer. Acc. Chem. Res. 53, 752-762 (2020).
23. Zhang, K. Y., et al. Long-lived emissive probes for time-resolved photoluminescence bioimaging and biosensing. Chem. Rev. 118, 1770-1839 (2018).
24. Gu, Y. Y., et al. High-sensitivity imaging of time-domain near-infrared light transducer. Nat. Photonics 13, 525-531 (2019).
25. Xu, G., et al. New generation cadmium-free quantum dots for biophotonics and nanomedicine. Chem. Rev. 116, 12234-12327 (2016).
26. Heiden, M. G. V., Cantley, L. C. & Thompson, C. B. Understanding the Warburg effect: The metabolic requirements of cell proliferation. Science 324, 1029-1033 (2009).
27. Wang, Y. G., et al. A nanoparticle-based strategy for the imaging of a broad range of tumours by nonlinear amplification of microenvironment signals. Nat. Mater. 13, 204-212 (2014).
28. Zhao, T., et al. A transistor-like pH nanoprobe for tumour detection and image-guided surgery. Nat. Biomed. Eng. 1, 0006 (2017).

### Example 2—Synthesis of CuInSe₂ Nanotube Probes and Supplemental Results for Example 1

### Materials and Methods:

Chemical reagents. Se powder (100 mesh, 99.99%), indium chloridehydrate (InCl₃.4H₂O, 97%), sodium borohydride (NaBH₄, >98%), reduced glutathione (GSH, 99%), oleic acid and n-dodecanethiol were purchased from Sigma-Aldrich. 1,2-distearoyl-sn-glycero-3-phosphoethanolamine-N-[methoxy(poly-ethylene glycol)-2000] (DSPE-PEG2000) was purchased from Shanghai Ponsure Biotech, Inc. All chemicals and reagents were commercially available and used without further purification unless special statements.

Characterization instruments. Transmission electron microscopy (TEM) images were performed on the JEM-2100F electron microscope with an acceleration voltage of 200 kV, using a dedicated low-background holder and Cu-free Mo TEM grids. High-angle annular dark-field scanning TEM (HAADF-STEM) and energy dispersive spectroscopy (EDS) were obtained on a FEI Titan Cubed Themis G2 300 with a probe corrector and operated at 60 kV. Atomic force microscopy (AFM) was conducted on the MultiMode 8 atomic force microscopic (Bruker, USA) operating in tapping mode in air with an MSNL-10 F #probe (Bruker, USA, spring constant: 0.5 N m⁻¹). PL spectra and decay curves were measured on an Edinburgh FLS1000 spectroscopy equipped with an 808 nm laser. To determine low-temperature PL spectra, CISe nanotubes were placed between quartz micro-glasses on a cold finger. The sample temperature was controlled using a helium closed-cycle cryostat (Janis, SHI-4-1) equipped with cooling helium-compressor (Sumitomo, CAN-11 C). Transient absorption (TA) was performed by a femtosecond pump-probe system (Transient Absorption Spectrometer, Newport Corporation). UV-Vis-NIR absorption spectra were measured on Carry 5000 spectrophotometer (Agilent Technologies, USA). Malvern Zetasizer Nano ZSP instrument equipped with Malvern surface zeta potential cell was used to measure zeta potential. X-ray photoelectron spectrometry (XPS) measurement was carried out by Thermo ESCALAB 250 spectrometer. Inductively coupled plasma spectroscopy (ICP) instrument (PerkinElmer Optima 8000) was used to measure [Cu]:[In]:[Se] atom ratios. The samples were prepared by dissolving them in HNO₃ and then diluted in deionized water. To determine the concentration of samples, a set of standards (0.1-50 ppm) were prepared using a RICCA 1000 ppm of copper, indium and selenium in 3% HNO₃ standard stock solutions.

Synthesis of CISe nanotubes. CISe nanotubes were prepared by a modified two-step procedure, i.e. synthesis of Cu₂₋ₓSe nanorods by water-evaporation-initiated self-assembly method followed by NaBH₄-reduction involved cation exchange resulting in CISe nanotubes.¹ The synthesis of Cu₂₋ₓSe nanorods was as follows. 0.15 g of Se powder, 0.25 g of GSH, 5.0 g of NaOH and 20 mL of distilled water were put in a 100 mL beaker with heating and stirring to dissolve Se powder. 5 mL of Cu(NO₃)₂ aqueous solution (1 M) was added, and the beaker was kept in fan-forced oven at 140° C. for 6 h. The Cu₂₋ₓSe product was collected and washed with hot water and ethanol for several times. In the next synthetic procedure, InCl₃.4H₂O (0.4 g) and 0.5 g of Cu₂₋ₓSe product were put in a Teflon-lined stainless-steel autoclave containing 30 mL of NaBH₄ solution (0.1 M), and then maintained at 210° C. for 1.5 h. After that, CISe nanotubes were collected and washed with water for several times, and then stored in drying condition for long-term preservation.

Synthesis of PEG-CIS nanorods as a control NIR-II fluorescent nanoprobe. In order to evaluate the superiority of NIR-II phosphorescence over NIR-II fluorescence in bioimaging, it is necessary to prepare NIR-II fluorescent probe with comparable shape, size and chemical composition to CISe nanotubes. In this context, we synthesized copper indium sulfide (CIS) nanorods as a control probe, following a procedure reported previously with a slight modification.² In a three-neck flask, 0.34 mmol of Cu(NO₃)₂ and 0.34 mmol of InCl₃.4H₂O were mixed in the solution of 5 mL of oleic acid and 5 mL of n-dodecanethiol, heated to 240° C. and kept there for 30 min via vigorous magnetic stirring. After that, the mixture was cooled to room temperature and precipitated with ethanol and centrifuged at 8000 rpm for 5 min to obtain the product, which can be easily re-dispersed in chloroform. The obtained oleic acid-coated CIS nanorods were hydrophobic. DSPE-PEG2000 was then utilized to stabilize the CIS nanorods. Firstly, DSPE-PEG2000 was added into chloroform solution (10 mL) containing oleic acid-coated CIS nanorods with a mass ratio of 10:1 for 10 min by sonication. Then, the mixture was heated up to 60° C. under vacuum in a rotary evaporator for 30 min to evaporate chloroform. Finally, 10 mL of PBS was added and sonicated for 2 min. The obtained PEG-CIS nanorods was stored at 4° C. for later use.

PL activation experiments. To study pH-triggered PL variation, CISe nanotubes (5 mg mL⁻¹) were dispersed in deionized water. Then, the solution was diluted in 50 mM PBS buffers with different pH values. The final concentration of CISe nanotubes was controlled at 0.1 mg mL⁻¹. The nanoprobe was excited at 808 nm, and PL spectra were collected from 900 to 1400 nm. The emission intensity at 1130 nm was used to quantify the signal amplification for CISe nanotubes. NIR-II images of CISe nanotubes (0.1 mg mL⁻¹) in different pH values were captured on intensity-based NIR-II imaging system and time-resolved NIR-II imaging system, respectively. The above procedure was also applicable to evaluate pH-triggered PL variation of PEG-CIS nanorods except recording emission intensity at 1035 nm. In order to study concentration-dependent switching of emission mode, CISe nanotubes dispersed in pH 6.8 PBS with a series of concentrations (e.g., 0.01, 0.1, 0.2, 0.5, 1, 2, 3 and 5 mg mL⁻¹) were prepared and the PL characterization method was identical to above procedure used in detecting pH-triggered PL variations.

PL stability and reversibility of CISe nanotubes. CISe nanotubes in fetal bovine serum (FBS) (pH 6.0, 0.1 M NaCl), PBS (pH 6.0, 0.1 M NaCl) and PBS (pH 6.0, 1.0 M NaCl) were loaded in 10 mm path length sealed quartz cuvettes, respectively. The samples were continuously illuminated at 808 nm with a power density of 0.14 W cm⁻² and their PL intensity of 1130 nm band was recorded every 10 s over a period of 100 min, while all the instrumental conditions maintained constant. NIR-II intensity images were collected at 0, 30 and 100 min. The cycling experiment of CISe nanotubes in deionized water (0.5 mg mL⁻¹) was carried out between pH 6.0 and pH 7.4 using 0.1 M HCl and NaOH. The emission intensity at 1130 nm was used to quantify the signal reversibility.

Acid-base titration experiment. The pKₐ of CISe nanotubes was measured as a function of pH using an acid-base titration method.³ In a typical procedure, a given amount of CISe nanotubes was dispersed in 6 mL of 50 mM HCl solution to enable full protonation. 5 μL aliquots of 0.1 M NaOH were added. Five minutes after each addition of NaOH, the pH was measured by a pH electrode (note: the pH did not change significantly after about 30 s). In order to prevent carbonate interference, the titration was performed within 30 min after solution preparation. The titration was repeated on a second sample to check for reproducibility.

### Results and Discussion

CISe nanotubes were prepared by a modified two-step procedure, namely water-evaporation-driven self-assembly way followed by the NaBH₄-reduction involved cation exchange process.⁴ Accordingly, there were two crucial aspects that should be considered: (1) synthesis of Cu₂₋ₓSe nanorods acting as a template to form ternary CISe nanotubes; (2) using partial cation exchange reaction to generate hollow structures. The rationale behind this two-step synthesis procedure was the separation of chemical pathways during morphological and structural evolutions. It will thus largely inhibit detrimental cross-interferences and side-reactions, such as Ostwald ripening in the stage of the formation of CISe nanotubes.

Using the water-evaporation-induced self-assembly approach, Cu₂₋ₓSe bundles that composed of one-dimensional nanorods with average diameters of 11.5 nm were observed (FIGS. 2.1A and 2.1B). Taking a closer look at each Cu₂₋ₓSe nanorod, we proved that it maintained intact solid microstructures. More crystallographic details were identified by HR-TEM. Lattice fringes with a constant spacing of 0.22 nm matched well with the spacing of CuRSe (220) plane, clearly indicating a preferential growth of nanorods along a [110] direction (see FIG. 2.1B).¹ Strategically, the prepared high-quality solid Cu₂₋ₓSe nanorods (e.g., uniform size distribution, high crystallinity and mass production) played an important role as a superior starting platform for subsequent preparation of the ternary CISe nanotubes.

The typical EDS spectrum of Cu₂₋ₓSe nanorods was presented in FIG. 2.1C. Also, EDS and ICP data were listed in Table 1 as follows. Analysis of mean composition by EDS suggested a [Cu]:[Se] ratio of 1.8:1 for parent CuRSe nanorods. Notably, ICP also presented a similar atomic ratio for Cu₂₋ₓSe nanorods. Thus, we can quantify the starting Cu₂₋ₓSe nanorods as Cu_{1.8}Se nanorods.

After reacting Cu_{1.8}Se nanorods with InCl₃ in a NaBH₄ alkaline solution, CISe nanotubes could be successfully obtained (FIGS. 2.2A-2.2C). In order to clarify valence states of element Cu, In, Se in the CISe nanotubes, XPS analysis was performed and the binding energy of Cu2p_{3/2}, In3d_{5/2} and Se3d was 931.6, 444.1 and 53.9 eV, respectively (FIG. 2.2A). These XPS results revealed that the chemical valence of element Cu, In and Se in CISe nanotubes were Cu⁺, In³⁺ and Se²⁻, respectively, according to the reference.⁵ In line with the above convincing results, we established that starting with binary Cu_{1.8}Se nanorods, the desired trivalent cation (i.e. In³⁺) was capable of incorporating into nanocrystal lattice by replacing partial host Cu⁺ cations during the solvothermal treatments.⁶ Under the solvothermal condition, it has been well documented that NaBH₄ plays an important contribution as reducing agent to reduce cupric ion (Cu²⁺) to cuprous ion (Cu⁺).⁷ As a consequence, no characteristic satellite peak around 942 eV was detected in the high-resolution XPS spectra (FIG. 2.2A), implying the absence of Cu²⁺ in CISe nanotubes.

The successful preparation of CISe nanotubes was evidenced by other molecular and structural characterizations, for example FT-IR and XRD. Disappearance of a peak at 2503 cm⁻¹, which was ascribed to the S—H stretching vibration belonging to GSH (FIG. 2.2B), revealed the reaction mechanism. Briefly, the thiol groups from GSH molecules, having intrinsic coordination capability,⁸ could chemically cap onto the CISe nanotubes as schematically illustrated in FIG. 1.1A (Example 1). Moreover, all the typical diffraction patterns in FIG. 2.2C can be precisely indexed to those of tetragonal CuInSe₂ (JCPDF: 75-0107) without any phase peaks of Cu_{R}Se nanocryatals.⁹

TEM and HR-TEM images remarkably demonstrated that the obtained CISe nanotubes inherited representative morphology (i.e. one-dimensional structure) from starting Cu_{1.8}Se nanorods (FIG. 1.1C in Ex. 1). By collecting intermediates at different cation exchange durations, HR-TEM was used to study the formation mechanism of hollow nanostructure in the CISe products (FIG. 2.3A). More importantly, the morphology of intermediates could be served a basis to optimize reaction times. The whole size of cavity gradually increased with an extension of reaction time. Besides, an interesting shape evolution of cavity was found to show hexagonal outlines at 0.5 h, a mixture of interconnected hexagonal and elongated structures at 1.0 h and a solely one-dimensional hollow void at 1.5 h. However, further replacement of Cu⁺ with In³⁺ will cause dismantling of original structures. The intermediates collected from a prolonged reaction time presented irregular shape (FIG. 2.3E), although sporadic CISe nanotubes with much thinner wall thickness can be detected (marked by dotted red line). As a consequence, optimized reaction time of cation exchange appeared to be about 1.5 h, and in further studies 1.5 h reaction time was selected to prepare the CISe nanotubes. Under this reaction time, cation exchange reaction presented a topotactic transformation, in which anion frameworks remained virtually intact. Hence, the morphology of overall one-dimensional structure could be generally retained.

Subsequently microarea analysis was exploited at a domain surrounding the formed hollow space to measure the structural constituents of the CISe nanotubes. Taken together, no matter what morphological types of cavity generated, In₂Se₃-rich inner crystal phases unambiguously existed throughout the whole wall of CISe nanotubes, as depicted with the dashed lines in the insets of FIGS. 2.3A-2.3C. HR-TEM images also hinted that such discernible borderlines between In₂Se₃ and CuInSe₂ nanocrystallites proceeded outward as the reaction time prolonged, leading to a steady expansion of In₂Se₃ region accompanied by downsizing of CuInSe₂ nanocrystallites.

In virtue of structural and constituent evolution of CISe nanotubes, we postulated the hollowing mechanism from solid Cu_{1.8}Se nanorods as follows (FIG. 2.3D). (1) The emergence of cavities in solid Cu_{1.8}Se nanorods was critically dependent on the cation exchange, facilitated by solvothermal treatments. The cation exchange reactions naturally involve diffusive motion of at least two types of cation species.¹⁰ It is interesting to mention that In³⁺ incorporation and Cu⁺ extraction usually happen with a comparable diffusion rate, mainly due to the similar ionic radii of In³⁺ (80 μm) and Cu⁺ (77 μm).¹¹ To remain anionic lattice frameworks intact, the outgoing Cu⁺ cations will diffuse in principle much more quantities than ingoing In³⁺ cations, i.e. replacement of more host Cu⁺ cations with less guest In³⁺ cation in nanocrystal structure. In this regard, a net outgoing mass flow accompanied interdiffusion event, which was balanced with a considerable, opposite flow of in-situ formed vacancies. Importantly, within the small volume of transforming nanocrystallites, these supersaturated vacancy clouds were likely to condense into voids, which possessed tendency to coalesce and to yield a hollow structure.⁴

By controlling the reaction parameters, we had successfully synthesized PEG-CIS nanorods as evidenced by TEM images (FIGS. 2.4A-2.4B). Their average length and diameter were 118 and 14 nm, comparable to the sizes of CISe nanotubes with average length and diameter of 170 and 12 nm. This feature would make PEG-CIS nanorods very helpful as a control nanoprobe when evaluated the superiority of NIR-II phosphorescence over NIR-II fluorescence for in vivo imaging. Meanwhile, a typical HR-TEM image of PEG-CIS nanorods was displayed in FIG. 2.4B. The interspace of lattice fringes of the lying nanorods was about 0.33 nm, corresponding to the (002) plane of wurtzite CuInS₂ nanocrystals.² It suggested the as-obtained PEG-CIS nanorods grew along the [001] direction.

In order to provide more evidences on the feasibility of long-lived phosphorescence for imaging applications, photophysical properties of CISe nanotubes and PEG-CIS nanorods in various PBS buffers were also studied by intensity-based NIR-II imaging systems. The imaging and photophysical results of PEG-CIS nanorods were summarized in FIGS. 2.5A-2.5D. The PL intensity of CISe nanotubes and PEG-CIS nanorods gave totally different pH-responses. The relative PL brightness was facilely monitored by imaging the vials with InGaAs camera under an 808 nm excitation and 1000 nm long-pass optical filter (FIGS. 2.5A, 2.5C). In a sharp contrast to pH-dependent PL activation of CISe nanotubes, PL intensity of PEG-CIS nanorods intuitively remained constant when pH values varied from pH 8.0 to 5.5. This pH-independent response indicated a “always on” feature of PEG-CIS nanorods, whereas this PL brightness was remarkably inferior to that of CISe nanotubes in acidic PBS (FIG. 1.2B in Example 1).

The PL of CISe nanotubes in pH 5.5 behaved in a triple-exponential decay manner, presenting a lifetime of 336.1 μs (see FIG. 1.2J in Ex. 1). In contrast, CIS nanorods as a control nanoprobe decayed double-exponentially with a mean lifetime of 257.9 ns (FIG. 2.5D). Such a big difference in lifetimes could be clearly distinguished through time-resolved NIR-II imaging systems (FIG. 2.5B and FIG. 1.2H in Ex. 1). For CISe nanotubes, decreasing the pH values from 8.0 to 5.5 significantly elongated the lifetimes. For example, lifetime signals of CISe nanotubes cannot be differentiated against background at pH 8.0 and 7.4, whereas the PL lifetime imaging at pH 6.5 and 5.5 recognized EP tubes from background interference. In contrast, the lifetime signals were hardly observable for CIS nanorods, irrespective of the pH values, ascribing to their much shorter lifetimes of hundreds of nanoseconds (FIG. 2.5D). The above time-resolved NIR-II imaging analysis indicated the utilization of long-lived phosphorescence for imaging by time-resolved imaging system was particularly promising in minimizing signal interferences with nanoseconds-scaled lifetime.

With respect to two facts that the pH of solution highly responsible for the stability of amino or carboxyl-bearing nanoparticles,¹² and aggregation-initiated changes in photophysical properties of various compounds,¹³ we utilized the classic acid-base titration method,¹⁴ a powerful tool for studying the stability of charged nanoparticles, to determine the pKₐ of CISe nanotubes. The pKₐ value was measured as 6.87 (FIG. 2.7A), which indicated a pKₐ around 4.0 pH units higher than that of free, unbound GSH molecules with an average isoelectric point of 2.9.¹⁵ This positive pKₐ shift was concordant with previous studies of terminal carboxyl moieties stabilized inorganic nanoparticles.¹⁴ Herein, the origin of pKₐ increase could be ascribed to an unfavorable accumulation of negative charge on surface of CISe nanotubes as the deprotonation reaction of carboxyl groups proceeded.³ The extent of pKₐ shift will depend perceptibly on the curvature of nanoparticles. When the size of nanoparticles decreases, the head groups of deprotonated GSH ligands, bearing a big average distance, would develop weaker electrostatic repulsion with each other. As a consequence, smaller nanoparticles manifested a slight positive shift in pKₐ.

The slightly acidic apparent pKₐ of 6.87 revealed that in alkaline or even in neutral solutions, the GSH ligands existed primarily in the negatively charged state. For example, the zeta potential of CISe nanotubes in pH 7.0 PBS was about −34.5 mV as shown in FIG. 2.7B. The CISe nanotubes remained electrostatically stabilized. With gradually declining pH, the surface charge of CISe nanotubes was decreased, making CISe nanotubes lose sufficient colloidal stability once carboxyl groups of GSH become protonated. Thus, temporal stability of CISe nanotubes quickly deteriorated to some extent upon further lowering pH to pH 5.0 with a zeta potential value of about −12.1 mV.

The self-limited assembly of GSH-capped CISe nanotubes might be explicitly rationalized by the following two effects (FIG. 2.9 and FIG. 1.2F in Example 1). First, decreasing surface charges made CISe nanotubes lose sufficient colloidal stability when the carboxyl groups of GSH ligands became protonated. A second and likely more vital effect was the favorable multiple H-bonding interactions between interparticle zwitterionic GSH that could drive association of CISe nanotubes into large compact clusters. In our case, the assembly behavior was possibly modulated via the delicate interplays between electrostatic repulsion and multiple H-bonding attraction,¹⁶ which preferentially achieved compact arrangement of CISe nanotubes by self-limited assembly against uncontrolled growth.

Taking CISe nanotubes in pH 5.5 as an example (FIG. 2.15E), the detailed calculation processes of phosphorescence lifetime were shown as follows.

There were three different decay functions with single-exponential, double-exponential and triple-exponential. To identify which decay functions was more suitable for the measured decay curves, the above three decay functions will be employed. The fit parameters were then refined until we could achieve an acceptable fit. The quality of the fit was judged by how closely the calculated decay (produced from an assumed exponential decay law) and the recorded PL decay matched in a least-squares sense. Meanwhile, another critical consideration was that the goodness-of-fit parameter was characterized by the value of Adj.R-Square. The closer the value of Adj.R-Square was to 1, the more suitable the decay function would be. Considering such two crucial criterions, we believed that the phosphorescence of CISe nanotubes in pH 5.5 decayed triple-exponentially with a substantially longer lifetime (FIG. 2.15E).

The functions of triple-exponential decay and average lifetime are:

$$I ( t ) = {α}_{1} exp ( - \frac{t}{{τ}_{1}} ) + {α}_{2} exp ( - \frac{t}{{τ}_{2}} ) + {α}_{3} exp ( - \frac{t}{{τ}_{3}} ) \\ {τ}_{a v g} = \frac{{α}_{1} {τ}_{1}^{2} + {α}_{2} {τ}_{2}^{2} + {α}_{3} {τ}_{3}^{2}}{{α}_{1} {τ}_{1} + {α}_{2} {τ}_{2} + {α}_{3} {τ}_{3}}$$

where α₁, α₂ and α₃ are the fractional contributions of the emission decay lifetimes of τ₁, τ₂, and τ₃, respectively. The emission intensity at zero time is assumed to be a unity, i.e., α₁+α₂+α₃=1.

$${α}_{i} = \frac{{A}_{i} {τ}_{i}}{Σ {A}_{i} {τ}_{i}}$$

where αᵢ is the fractional contribution of each decay component to the steady intensity I₀. The denominator is over all amplitudes and decay times which is proportional to the total intensity.¹⁷ The Aᵢ is obtained directly from the decay functions by the Origin software.

The triple-exponential decay equation including lifetimes and their related parameters could be obtained by the Origin software:

$$\begin{matrix}I ( t ) = {A}_{1} exp ( - \frac{t}{{τ}_{1}} ) + {A}_{2} exp ( - \frac{t}{{τ}_{2}} ) + {A}_{3} exp ( - \frac{t}{{τ}_{3}} ) \\ = 1.33 \; 5 exp ( - \frac{t}{0.0307} ) + 0.357 exp ( - \frac{t}{0.0278} ) + \\ 0.02 \; 81 exp ( - \frac{t}{0.4277} )\end{matrix}$$

$$i . e . {A}_{1} = 1.335 , {τ}_{1} = 0.0 \; 307 ms$$

$${A}_{2} = 0.357 , {τ}_{2} = 0.0 \; 278 ms$$

$${A}_{3} = 0.0281 , {τ}_{3} = 0.4 \; 277 ms$$

The fractional contribution of αᵢ need to be calculated:

$${α}_{1} = \frac{{A}_{1} {τ}_{1}}{\sum {A}_{i} {τ}_{i}} = \frac{1.335 \times 0.0307}{1.335 \times 0.0307 + 0.357 \times 0.0278 + 0.0281 \times 0.4277} = 65.1 \% \\ {α}_{2} = \frac{{A}_{2} {τ}_{2}}{\sum {A}_{i} {τ}_{i}} = \frac{0.357 \times 0.0278}{1.335 \times 0.0307 + 0.357 \times 0.0278 + 0.0281 \times 0.4277} = 15.8 \%$$

$${α}_{3} = \frac{{A}_{3} {τ}_{3}}{\sum {A}_{i} {τ}_{i}} = \frac{0.0281 \times 0.4277}{1.335 \times 0.0307 + 0.357 \times 0.0278 + 0.0281 \times 0.4277} = 19.1 \%$$

The average lifetime of T_{avg} then can be calculated below:

$$\begin{matrix}{τ}_{a v g} = \frac{{α}_{1} {τ}_{1}^{2} + {α}_{2} {τ}_{2}^{2} + {α}_{3} {τ}_{3}^{2}}{{α}_{1} {τ}_{1} + {α}_{2} {τ}_{2} + {α}_{3} {τ}_{3}} \\ = \frac{65.1 \% \times 0.030 {7}^{2} + 15.8 \% \times 0.027 {8}^{2} + 19.1 \% \times 0.427 {7}^{2}}{65.1 \% \times 0.0307 + 15.8 \% \times 0.0278 + 19.1 \% \times 0.4277} \\ = 0.336076 ms \\ = 336.1 μs\end{matrix}$$

The following were the detailed calculation processes of the phosphorescence lifetime of CISe nanotubes in pH 7.2 (FIG. 2.15A).

By the Origin software, the double-exponential decay equation including lifetimes and their related parameters could be obtained:

$$\begin{matrix}I ( t ) = {A}_{1} exp ( - \frac{t}{{τ}_{1}} ) + {A}_{2} exp ( - \frac{t}{{τ}_{2}} ) \\ = 0.66 \; 178 exp ( - \frac{t}{0.05892} ) + 0.57462 exp ( - \frac{t}{0.06033} )\end{matrix}$$

$$i . e . {A}_{1} = 0.66178 , {τ}_{1} = 0.0 \; 5892 ms$$

$${A}_{2} = 0.57462 , {τ}_{2} = 0.0 \; 6033 ms$$

The fractional contribution of αᵢ needed to be calculated:

$${α}_{1} = \frac{{A}_{1} {τ}_{1}}{\sum {A}_{i} {τ}_{i}} = \frac{0.66178 \times 0.058 \; 92}{0.66178 \times 0.05892 + 057462 \times 0.06033} = 52.9 \% \\ {α}_{2} = \frac{{A}_{2} {τ}_{2}}{\sum {A}_{i} {τ}_{i}} = \frac{0.57462 \times 0.0603 \; 3}{0.66178 \times 0.05892 + 057462 \times 0.06033} = 47.1 \%$$

The average lifetime of T_{avg} then can be calculated below:

$$\begin{matrix}{τ}_{a v g} = \frac{{α}_{1} {τ}_{1}^{2} + {α}_{2} {τ}_{2}^{2}}{{α}_{1} {τ}_{1} + {α}_{2} {τ}_{2}} \\ = \frac{52.9 \% \times 0.0589 {2}^{2} + 47.1 \% \times 0.0603 {3}^{2}}{52.9 \% \times 0.05892 + 47.1 \% \times 0.06033} \\ = 0.059592 ms \\ = 59.6 μs\end{matrix}$$

In addition to the standard decay analysis method (i.e. nonlinear least-squares method) as shown in FIGS. 2.14A-2.14F, FIGS. 2.15A-2.15E, the PL decay curves can be analyzed by the Higashi-Kastner approach (FIGS. 2.16A-2.16F, FIGS. 2.17A-2.17E). Generally speaking, this approach is based on the fact that the function I_{PL}(t)×(PL decay signals multiplied by the delay time) reveals a distribution of delay times. The peak information (e.g., the peak numbers and positions) of this function can be taken as the dominant decay time of PL distribution.^{18,19}

FIGS. 2.16A-2.16F and FIGS. 2.17A-2.17E showed the decay time distribution reconstructed from PL decay profiles detected at 1010 and 1130 nm for CISe nanotubes in various PBS buffers. By analysis of the 1010 nm-decay profiles of CISe nanotubes in pH 6.8, 6.5 and 5.5, two lifetime components were found (FIGS. 2.16A-2.16F). For example, a bimodal decay time distribution was observed at τₘₐₓ=0.19 μs and τₘₐₓ=1.42 μs for CISe nanotubes in PBS (pH 5.5). Especially, the decay time distribution analysis indicated additional slow decay components centered at τₘₐₓ=0.7-1.5 μs range coming up with declining pH (FIGS. 2.16A-2.16F). The 1130 nm-decay profiles of CISe nanotubes were also treated by the Higashi-Kastner approach (FIGS. 2.17A-2.17F). As clearly indicated with the black solid arrows, the slow decay times were clearly observed. The longest lifetime (Σ₃) of CISe nanotubes in pH 6.8 was about 334.5 μs, while it increased to 472.2 μs at pH 5.5. Importantly, by careful comparison of above two analysis methods, the PL decay curves analyzed by the Higashi-Kastner method (FIGS. 2.16A-2.16F, 2.17A-2.17F) showed very similar fitting results by the nonlinear least-squares fitting method (FIGS. 2.14A-2.14F, 2.15A-2.15E).

As schematically depicted in FIG. 1.31 in Ex. 1, the presence of Cu-rich state could allow the migration of abundant singlet excitons from high-lying S₁ state to triplet excitons in In-rich state. The process was further investigated by low-temperature photoluminescence spectra and the results were displayed in FIGS. 2.19A-2.19C. Understanding the relaxation dynamics of the photoexcited CISe nanotubes was an important step towards achieving a full understanding of the generation mechanism of phosphorescence by the Cu-to-In exciton transfer. The solution of CISe nanotubes was carefully dropcast onto a clean quartz coverslip and allowed to dry. The obtained CISe powder was placed between quartz micro-glasses on cold finger, and the sample temperature was tuned by helium closed-cycle cryostat (Janis, SHI-4-1) equipped with a cooling helium-compressor (Sumitomo, CAN-11 C). The typical temperature range of optical cryostat was set from 4 to 325 K, and the cooling power of cold head was set as 0.1 W at 4.2 K.

FIGS. 2.19A-2.19C presented temperature-dependent photoluminescence spectra for CISe nanotubes detected at temperatures from 4 to 300 K, which identified the presence of excitons transfer for the photoexcited CISe nanotubes. Notably, with increasing the temperature from 4 to 80 K, about 1.5-fold increase of PL intensity (FIGS. 2.19A, 2.19B) supported the exciton transfer dynamics as evidenced by TA spectra in FIG. 1.3 in Ex. 1. Singlet excitons created by photoexcitation could quickly transfer to low-lying In-rich states at elevated temperatures, a similar process to the internal conversion through thermal vibrational relaxation.²⁰ As a comparison, the PL peak positions of PEG-CIS nanorods (intrinsic defect-free crystal structures, FIG. 2.4B) redshifted from 972 to 986 nm (FIG. 2.20), which was not as drastic compared with CISe nanotubes. Hence, we preliminarily postulated the measured red shift of temperature-dependent PL spectra of CISe nanotubes might originate from the intrinsic defects (lattice mismatching) as evidenced by the abundance of rhomboid In-rich regions within well-developed crystalline CuInSe₂ matrix (see FIG. 1.3G in Ex. 1). Similar phenomenon of temperature-dependent PL redshift had been reported by other scientists.^{21,22} For example, Prof. Hyun's group prepared Zn-doped CIS nanocrystals and identified the lattice mismatching between core and shell parts as the origin of PL spectra redshift with the increase of temperatures.²²

When the temperature was further increased from 80 to 300K, the emission intensity was only slightly decreased (FIG. 2.19B), mainly owing to the enhanced nonradiative decay pathways. Multiple lines of evidence in organic/inorganic systems over the past several decades have well documented the critical role of nonradiative transition probability induced by thermal activation in the decrease of emission intensity.^{20,22-25} According to the classical theory on thermal quenching, the relationships between the emission intensity and sample temperature could be described by an Arrhenius-type activation model as:

$${I}_{T} = \frac{{I}_{0}}{1 + A exp ( - Δ E / {κ}_{B} T )} \qquad ( 1 )$$

where I₀ is the initial luminescence intensity, I_{T} is the luminescence intensity at given temperature T, A is a constant, κ_{B} is Boltzmann constant and ΔE is the activation energy for the thermal quenching process.
Equation (1) was mathematically transformed into:

$$In ( \frac{{I}_{0} - {I}_{T}}{{I}_{T}} ) = - \frac{Δ E}{{κ}_{B}} \frac{1}{T} + InA \qquad ( 2 )$$

As a consequence, the plot of In(I₀−I_{T})/I_{T} against 1/T was linear displaying a slope −ΔE/κ_{B} and intercept InA. Using the above equation, a good fitting result was presented in FIG. 2.19C with a slope of about −150.7. Since the experimental results and theoretical fits were in relatively good agreement for the powder sample, the slight decline of emission intensity from 80 to 300 K was originated from nonradiative thermal quenching process. However, the value of ΔE was calculated as 3.9 eV at 300 K for the solid powder of CISe nanotubes. The ΔE of the as-prepared CISe nanotubes was significantly higher than those of most ternary colloidal I-III-VI₂ nanocrystals (e.g., copper indium sulfide and its heavier congener CISe or their metal-doped nanoparticles). For example, it has been reported that the values of ΔE were estimated to be about 0.1 eV for Cu_{0.2}InS₂ nanocrystal,¹⁹ about 0.06 eV for Mn-doped CuInS₂ nanocrystals,²¹ and over 0.2 eV for quaternary ZnAgInSe quantum dots.²⁶ The nonradiative relaxation process with a large activation energy resulted in a stable PL intensity responding to temperatures. It should be mentioned that the temperature stability of optical properties was considerably important for evaluating nanoprobes in biological applications, since the luminescent materials may suffer from complex triggers from external environments, including high temperature during the long-term operations.²⁴ This unique thermal stability would make as-prepared CISe nanotubes very exciting as a nanoprobe for biological depth imaging.

Based on the Rose criterion,²⁷ resistance to photobleaching can enhance temporal resolution of in vivo imaging by preventing the loss of dynamic biological information, especially in the case of continuously long-term bioimaging. As a result, before applying the newly fabricated CISe nanotubes acting as a nanoprobe for in vivo imaging, their photostability should be assessed. As shown in FIG. 1.2N in Ex. 1, the photobleaching was almost negligible by exposing the CISe nanotubes into high-salted PBS and FBS under a 100-min continuous 808 nm laser irradiation at power density of 140 mW cm⁻². Meanwhile, a superior stability in PL properties was established in warm PBS and FBS at 37° C. over a period of 500 min (FIG. 2.21), demonstrating long-term photostability of CISe nanotubes in diverse environments.

Moreover, the as-obtained CISe nanotubes were capable of undergoing highly reversible “on-off” intensity over many times toward a pH cycling between 6.0 and 7.4 (FIG. 1.2M in Ex. 1). Mainly stemming from pH-induced breakage and reconstruction of dynamic H-bonding network among carboxyl and amino groups in GSH, which gave rise to repeatable aggregation, the PL property of CISe nanotubes quickly returned back to their original states and benefited a good reversibility. On the basis of verified physiological pH-modulated NIR-II phosphorescence with good reversibility and photostability, CISe nanotubes were a particularly attractive nanoprobe for real-time, long-term in vivo tumor imaging.

FIG. 1.31 in Ex. 1 illustrated self-limited assembly driven stabilization of Cu-to-In exciton transfer that was established as the origin of fluorescence-to-phosphorescence switch for CISe nanotubes. Increasing evidence supported the introduction of defects as an efficient method to tailor PL positions and intensities of luminescent nanomaterials.²⁸ In the case of CISe nanotubes, Cu⁺-to-In³⁺ cation exchange reactions (FIG. 2.3) enabled the doping of In³⁺ to build efficient and stable Cu-to-In metallophilic interaction,²⁹ thereby allowing migration of abundant singlet excitons from high-lying S₁ states to triplet excitons in In-rich domains (FIG. 1.31 in Ex. 1). Very importantly, self-limited assembly-driven stabilization of Cu-to-In exciton transfer facilitated the relaxation of excitons via a radiative pathway, while strongly restricted vibration and rotation of ligands can suppress the recombination of excitons by a nonradiative relaxation method. In addition, Cu-to-In exciton transfer possibly provided additional, lower metal energy level, giving rise to a red-shift of PL bands.

Strategically, there were two extremes of control experiments to look into whether proposed assembly-induced stabilization of Cu-to-In exciton transfer as an origin of phosphorescence was feasible. As anticipated, the CISe nanotubes in solid powders were understood to be a positive extreme representing the utmost compact extent, which in principle can deliver high-quality phosphorescent emission. In fact, the solid powder showed an intense, narrow and symmetrical PL spectrum with a substantially long lifetime of 381 μs and high QY of 39.8% (FIGS. 2.10B and 2.22). The maximum peak increased to 1140 nm. Note that such slightly red-shifted emission coincided well with the lower-energy emission observed from the assembly of CISe nanotubes in solution (FIG. 1.2C in Ex. 1). On the opposing extreme was PEG-CIS nanorods. Negligible surface defects (FIGS. 2.4A, 2.4B) caused these nanorods lacking an opportunity to regulate their emission through exciton transfer, which in turn emitted relatively weak fluorescence (FIGS. 2.5A-2.5B). The union of above photophysical properties supported a convincing result that self-limited assembly driven stabilization of Cu-to-In exciton transfer played a paramount role of generating phosphorescence-type PL for CISe nanotubes in acidic conditions or solid powders.

The assembly behavior that could affect the switch of fluorescence to phosphorescence was further demonstrated by concentration-dependent emission spectra (FIG. 2.23A). In pH 6.8 solution with the concentrations of CISe nanotubes less than 0.1 mg mL⁻¹, the solution delivered a single, faint emission peak at 1010 nm. When concentrations continually increased to 3.0 mg mL⁻¹, one new NIR-II peak appeared and developed at about 1130 nm, while the PL intensity of original peak at 1010 nm decayed evidently. Further elevating concentration of CISe nanotubes intensified their NIR-II emissions and became exclusively dominant at 5.0 mg mL⁻¹. NIR-II images of CISe nanotubes with an 808 nm excitation with various long-pass optical filters (FIG. 2.23B) afforded additional interesting demonstrations of the concentration-responsive bimodal emission. No light can be emitted from CISe nanotubes with concentrations below 0.2 mg mL⁻¹ under a 1100 nm filter, as opposed to apparent brightness detected using a 950 nm filter. This discrimination obtained by optical filter was not applied into CISe nanotubes with higher concentrations from 1.0 to 5.0 mg mL⁻¹. It was noteworthy to specify that at 5.0 mg mL⁻¹, the PL intensity of CISe nanotubes under 1100 nm filter was as pronounced as intensity showed under 950 nm filter, whereas a decline tendency could be seen at lower concentrations. Such exciting data suggested that at high concentrations of CISe nanotubes, corresponding to a compact arrangement, NIR-II signal at 1130 nm played a dominating contribution in PL intensity. Interestingly, concentration-dependent bimodal emission and switch only appeared in CISe nanotubes rather than PEG-CIS nanorods (FIGS. 2.24A-2.24C).

For preliminary evaluation of biocompatibility of CISe nanotubes, the in vitro cell viability assays (FIGS. 2.25A-2.25C) were performed. Cell viabilities remained at high levels and no obvious suppression can be monitored. For example, at a high concentration of 800 μg mL⁻¹, both the cell viabilities after 24 h and 48 h of incubation were approximately 100%, thus demonstrating minimal cytotoxicity associated with CISe nanotubes.

To assess the influence of nanotubes on the development and growth of mice, body weight was continuously recorded. Each mouse was injected with 100 μL of buffered saline or buffered CISe nanotubes (dosage: 100 mg kg⁻¹) by tail vein. Following these injections, the mice were weighed at different time points from 0 to 30 days. As shown in FIG. 2.26, there was no significant difference in average body weights after injection of CISe nanotubes over the 30-day period, accompanying with the weight gain of about 0.23 g day⁻¹. More importantly, the behaviors and appearance of mice after intravenous injection of CISe nanotubes were also monitored continuously. No significant differences in eating, drinking, hair color and glossiness can be found between the CISe nanotubes-treated mice and the control groups. Meanwhile, no aggression or unusual responses were observed in the treated mice compared with the control group. All these preliminary results indicated that the CISe nanotubes will not cause overall side effects on the mice.

As clearly shown in FIGS. 2.27A-2.27B, NIR-II phosphorescence could make CISe nanotubes very promising for the through-skin and in vivo real-time tumor vascular imaging. Serial high-magnification NIR-II intensity images vividly revealed that CISe nanotubes as an intravenous probe gradually leaked from chaotic, tortuous vascular systems (typical aberrant characteristics of tumor vasculature) and subsequently diffused into the surrounding tumor tissues, which can be attributed to the structural abnormalities and permeability increase of tumor vasculatures.³⁰ Generally, CISe nanotubes that were initially restricted into tumor vessels remained “PL silent”, whereas the probes after diffusing into interstitial space of tumor parenchyma and assembling to a high degree could light the tumor vasculatures up. For example, as the permeability of CISe nanotubes proceeded further toward extracellular milieu after 24 h post-injection, acidic tumor microenvironment was sufficient to nonlinearly activate PL signal of CISe nanotubes and in turn illuminated the whole tumor tissues. Based on through-skin, in vivo real-time imaging of tumor vasculatures, long-wavelength phosphorescence of CISe nanotubes with nonlinear amplification of NIR-II signal at the tumor tissue facilitated resolving tumor vessels with a spatial resolution of about 7.6 μm (signal-to-noise ratio ˜16.3) at above 1 mm (skin thickness) of penetration depth (FIG. 1.5J in Ex. 1), suggesting a high spatial resolution of NIR-II phosphorescence imaging.

Quantitative characterizations by cross-sectional PL intensity profiles of FIGS. 1.5C and 1.5D in Ex. 1 were displayed in FIGS. 2.28A-2.28F. The quantitative analysis clearly indicated that CISe nanotubes emitting NIR-II phosphorescence allowed much better tumor imaging than PEG-CIS nanorods emitting NIR-II fluorescence based on the following three aspects. First, the NIR-II signal intensities in tumor tissues were measured by the cross-sectional intensity profiles of the same location marked by green-dashed lines. Compared with the signal intensities at 24 h post-injection, CISe nanotubes-injected tumor mice showed about 1.44-fold higher than that of PEG-CIS nanorods. Second, tumor-to-normal tissue (T/NT) ratios from CISe nanotubes-treated tumor mice proved to be significantly higher than that detected by PEG-CIS nanorods (FIGS. 2.28C and 2.28F; 190.2 vs 14.6). Third, taking a closer look at cross-sectional intensity profiles of tumor tissue, the full-width at half-maximum (FWHM) of representative NIR-II images obtained by CISe nanotubes was ˜0.8 cm, whereas this value estimated from PEG-CIS nanorods-injected mice increased to ˜2.4 cm (clearly larger than the actual sizes of tumor, see FIGS. 2.30A-2.30C). Overall, the above results indicated that CISe nanotubes with NIR-II phosphorescence could provide high-precision tumor imaging and present more accurate information on tumor tissues compared with imaging by NIR-II fluorescent nanoprobes.

To explore the pH specificity of as-prepared CISe nanotubes, we utilized two inhibitors of tumor glycolysis to establish proof of principle for acidic extracellular pHₑ-induced high tumor imaging. As depicted in FIG. 1.5F in Ex. 1, the first agent of 2-deoxy-D-glucose (2-DG) inhibits glucose uptake by cell surface glucose transporter and could simultaneously reduce phosphorylation via hexokinases. Another agent, α-cyano-4-hydroxycinnamate (CHC),³¹ is a typical monocarboxylate transporter inhibitor that avoids lactic acid secretion from tumor cells.

Tumor cells are characterized by aerobic glycolysis, a phenomenon termed as “Warburg effect”, associating with increased lactate secretion.³² Deregulated pH is thus emerging as one hallmark of cancer, as tumors show a “reversed” pH gradient with a constitutively increased intracellular pH that is higher than the extracellular pH (pHₑ, 6.5-6.8).³³ As expected, the in vitro cell medium studies indicated that both the inhibitors significantly decreased lactate secretions in two types of cancer cells (FIGS. 2.29A, 2.29B). Accordingly, we found that 2-DG or CHC treatments markedly retarded acidification of 143B cell culture medium with ΔpH of 0.41, 0.08 and 0.10 for control, 2-DG and CHC, respectively (FIG. 2.29C). Interestingly, a similar inhibition could be detected in cell line (MCF-7 cells) representing other cancer types (FIG. 2.29D).

The finding that ultrahigh specific imaging for 143B tumors prompted us to explore the utility of CISe nanotubes for in vivo imaging another different tumor-bearing mouse model. Thereby, we prepared a model bearing subcutaneous tumors created with the MCF-7 cells on the upper rear leg of nude mice (images under white light, FIGS. 2.32A-2.32C). The tumor location was clearly different from that implanted by the 143B tumor cells (the right shoulder of the nude mice, see FIGS. 2.30A-2.30C). As clearly shown in FIG. 2.32A, the PL signal from PEG-CIS nanorods-administrated nude mice presented a gradual enhancement in the tumors over time, distinctly suggesting that PEG-CIS nanorods passively distributed into the tumors via EPR effect. As expected, PL signal was mainly confined to the mononuclear phagocyte systems organs (liver, spleen) on NIR-II images immediately captured after injection of PEG-CIS nanorods. At 24 h, substantial PL signals appeared inside the tumor as well as other nontarget tissues, such as intestine and heart. Note that a similar finding that PEG-CIS nanorods could produce unspecific signals was also identified by the mouse model-bearing 143B tumors at various implantation locations (FIG. 1.5D in Ex. 1 and FIGS. 2.30A-2.30C). These strong and ubiquitous background noises definitely lowered the sensitivity and specificity for tumor detection, and even caused “false positive” results.

In a sharp contrast to PEG-CIS nanorods-treated mice, when CISe nanotubes were intravenously injected into the mice (FIG. 2.32B), we could observe that the emission activation pattern by nanoprobes only occurred in the tumor microenvironment over surrounding normal tissues and organs. The PL intensity in tumor site that represented accelerated accumulation of CISe nanotubes by the EPR effect became evidently visible as the background maintained clean over time and more so after 24 h. Likewise, pretreating mice by either metabolic inhibitor could significantly result in the near-completely in vivo quenching tumor luminescence signals from CISe nanotubes (FIG. 2.32C). Such ultrahigh tumor-specific imaging supported that (1) CISe nanotubes can facilely distinguish the very small pH differences between acidic extracellular pH, (6.5-6.8) and blood (7.4); (2) this pH, in the tumor milieu was sufficiently acidic to activate the PL of CISe nanotubes, lighting tumor up in a “on” state. Taken together, the pH specificity studies highlighted that the success of targeting tumor acidic microenvironments by CISe nanotubes could serve as a more robust and universal strategy to achieve broad tumor specificity.

The following were the detailed calculation processes on how many Cu⁺ can be replaced by In³⁺ cations:

$$n = \frac{{[ Cu ]}_{starting} - {[ Cu ]}_{final}}{{[ In ]}_{final} - {[ In ]}_{starting}}$$

where n was the number of Cu⁺ replacing by the In³⁺ cation.

Quantification of EDS spectra (Table 1) showed a [Cu]:[In]:[Se] ratio of 3.68:0:2 for the starting Cu_{R}Se nanorods and 0.72:1.01:2 for the optimized CISe nanotubes (also see FIG. 2.3F).

$$n = \frac{3.68 - 0.72}{1.01 - 0} = 2.93$$

Likewise, quantification of the ICP spectra (Table 1) can confirm this calculation result. By ICP measurements, [Cu]:[In]:[Se] ratios were 3.88:0:2 for the starting CuRSe nanorods and 0.85:1.04:2 for the optimized CISe nanotubes.

$$n = \frac{3.88 - 0.85}{1.04 - 0} = 2.91$$

According to above experiment analysis, for the optimized CISe nanotubes prepared with cation exchange time of 1.5 h, we confirmed a replacement of approximately 2.9 host Cu⁺ with 1 guest In³⁺ cation in cation exchange.

TABLE 1
The apparent atom ratios of CISe nanotubes synthesized at different
reaction times of cation exchange. EDS and ICP measurements were
both utilized to determine the atom ratios of CISe nanotubes.

| Reaction | Element composition | | | | | | | |
|---|---|---|---|---|---|---|---|---|
| time of cation | EDS | | | | ICP | | | |
| exchange | Cu | In | Se | Formula | Cu | In | Se | Formula |
| 0 | 3.68 | 0.00 | 2 | Cu_{1.8}Se | 3.88 | 0.00 | 2 | Cu_{1.9}Se |
| 0.25 | 3.66 | 0.39 | 2 | Cu_{3.7}In_{0.4}Se₂ | 3.48 | 0.39 | 2 | Cu_{3.5}In_{0.4}Se₂ |
| 0.5 | 1.32 | 0.81 | 2 | Cu_{1.3}In_{0.8}Se₂ | 1.41 | 0.81 | 2 | Cu_{1.4}In_{0.8}Se₂ |
| 1.0 | 1.05 | 0.97 | 2 | Cu_{1.1}InSe₂ | 1.07 | 0.96 | 2 | Cu_{1.1}InSe₂ |
| 1.5 | 0.72 | 1.01 | 2 | Cu_{0.7}InSe₂ | 0.85 | 1.04 | 2 | Cu_{0.9}InSe₂ |
| 3.0 | 0.68 | 1.29 | 2 | Cu_{0.7}In_{1.3}Se₂ | 0.77 | 1.22 | 2 | Cu_{0.8}In_{1.2}Se₂ |

TABLE 2
Photophysical properties of CISe nanotubes dispersed in PBS buffers
with different pH and freeze-dried into solid powder, respectively.

| CISe | λₑₓ | λₑₘ | Lifetime (μs) | | | | QY |
|---|---|---|---|---|---|---|---|
| nanotubes | (nm) ^{a)} | (nm) ^{b)} | T₁ (α₁%) | T₂ (α₂%) | T₃ (α₃%) | Tₐᵥₑ | (%) ^{c)} |
| In pH 7.4 | 715 | 1010 | 0.07 (49.4) | 0.07 (50.6) | N/A ^{d)} | 0.073 | \ ^{e)} |
| In pH 7.2 | 712 | 1003 | 0.12 (99.9) | 1.20 (<0.1) | N/A | 0.117 | 0.63 |
| | | 1125 | 58.9 (52.9) | 60.3 (47.1) | N/A | 59.6 | 0.28 |
| In pH 7.0 | 712 | 1007 | 1.63 (<0.1) | 0.10 (99.9) | N/A | 0.099 | 0.87 |
| | | 1125 | 72.6 (79.1) | 82.4 (20.9) | N/A | 74.8 | 1.31 |
| In pH 6.8 | 710 | 1032 | 0.08 (99.9) | 0.60 (<0.1) | N/A | 0.076 | 4.64 |
| | | 1131 | 20.6 (77.4) | 340.5 (22.6) | N/A | 285.3 | 8.02 |
| In pH 6.5 | 710 | 1020 | 0.15 (99.9) | 1.47 (<0.1) | N/A | 0.154 | 5.12 |
| | | 1130 | 26.1 (49.0) | 30.8 (30.0) | 391.2 (21.0) | 314.2 | 9.61 |
| In pH 5.5 | 703 | 1030 | 1.25 (<0.2) | 0.20 (99.8) | N/A | 0.212 | 2.53 |
| | | 1138 | 30.7 (65.1) | 27.8 (15.8) | 427.7 (19.1) | 336.1 | 12.4 |
| Solid | 700 | 1140 | 25.8 (15.7) | 75.8 (17.3) | 401.4 (67.0) | 381.1 | 39.8 |

^{a)} From steady-state photoluminescence spectra upon emission at λₑₘ.
^{b)} From steady-state photoluminescence spectra upon excitation at 808 nm.
^{c)} The QY values of CISe nanotubes in PBS buffers (pH = 7.4, 7.2, 7.0, 6.8, 6.5 and 5.5) were measured by using a standard fluorophore of IR-26 (QY: 0.05%) as reference, while the QY of CISe nanotubes in solid powders was estimated using an integrating sphere (see the section: Measuring NIR-II quantum yield as follows).
^{d)} N/A represented “not applicable”, corresponding to the double-exponential decay mode for photoluminescence without T₃.
^{e)} The symbol “\” represented QY cannot be precisely calculated due to the weak PL signals.

### Measuring NIR-II Quantum Yield

Using a Standard Dye of IR-26 as Reference to Calculate Solution PL Quantum Yield

To measure the quantum yield (QY) of CISe nanotubes, a typical reference fluorophore (e.g., IR-26) that also emits in a NIR-II range was selected.³⁴ While there is some debate over the true QY of IR-26, 0.05% is a widely accepted value. The QY was calculated in the following equation:

$$QY = Q {Y}_{ref} \times \frac{{n}^{2}}{{n}_{ref}^{2}} \times \frac{s l o p {e}_{sample}}{s l o p {e}_{ref}}$$

Where the subscript ref denotes the reference fluorophore, slope is calculated from the plot of integrated fluorescence intensity versus UV absorbance and n represents the refractive index of the solvents (e.g., dichloroethane for IR-26 and water for CISe nanotubes). In order to decrease random error, five different concentrations of fluorophores are measured.

Using an Absolute Method to Measure Solid PL Quantum Yield

The QY of solid CISe nanotubes was estimated on the spectrofluorometer attached with a JASCO ILF-53 integrating sphere unit, according to a published protocol.³⁵

The QY of CISe nanotubes was measured in a similar method as reported previously.³⁶ Briefly, 1 mg mL⁻¹ IR-26 in 1,2-dichloroethane was diluted into a series of solutions with UV absorbance values at 808 nm of about 0.10, 0.08, 0.06, 0.04 and 0.02 (from top to bottom), respectively (FIG. 2.12A). A total of five IR-26 solutions with linearly spaced concentrations were placed into a 1-cm quartz cuvette one at a time. The PL spectra were also collected (FIG. 2.12C) and integrated. Then, area under curve was plotted against the UV absorbance value at 808 nm (FIG. 2.12E) and a linear fit was applied to indicate the slope. CISe nanotubes in PBS buffers at pH 5.5 with increasing concentrations were also measured at 808 nm to obtain UV absorbance values (FIG. 2.12B) and the PL emission spectrum was monitored following the manner specified above (FIG. 2.12D). Likewise, area under curve of PL emission spectrum for each CISe nanotubes was plotted against their 808 nm UV absorbance and fitted into a linear function, as slope was displayed (FIG. 2.12F). By comparing the slope of the linear fit between reference IR-26 and CISe nanotubes, the QY was calculated based on the above equation:

$${QY}_{CISe nanotubes i n p H 5.5} = 0.0 \; 5 \% \times \frac{1.3 {3}^{2}}{1.44 {8}^{2}} \times \frac{1953192648}{6664620} = 12.4 \%$$

For the calculation of QY of CISe nanotubes dispersed in other PBS solutions with pH 7.0 and 6.5, a similar protocol described above was followed, but the mixed PL spectra should be firstly separated (FIG. 2.13A-B). Accordingly, QY values of CISe nanotubes in different PBS buffers were listed in Table 2. We found that the QY values of as-prepared CISe nanotubes, especially in acidic solutions, were comparable or even higher than the majority of QY values of currently reported nanoprobes (Table 3).

TABLE 3
Comparison of lifetimes including their Stokes shift, emission types and quantum yield
(QY) of CISe nanotubes with other currently reported probes. Notably, the lifetimes
originating from phosphorescence were indicated by boldface, italicized text.

| | λₑₓ | λₑₘ | Stokes | Lifetime | QY | Emission | |
|---|---|---|---|---|---|---|---|
| Materials | (nm) ^{a)} | (nm) ^{b)} | shift | (μs) | (%) | type | Reference |
| [symbol] | [symbol] | [symbol] | [symbol] | [symbol] | [symbol] | [symbol] | [symbol] |
| [symbol] | | | | | | | |
| [symbol] | | | | | | | |
| [symbol] | [symbol] | [symbol] | [symbol] | [symbol] | [symbol] | [symbol] | [symbol] |
| [symbol] | | | | | | | |
| Donor-acceptor- | 780 | 1100 | 320 | — | 6.0 | F ^{d}) | Adv. Mater., 2020, |
| donor probes | | | | | | | 1907365 |
| Ag₂S | 808 | 1094 | 286 | — | — | F | Angew. Chem. Int. Ed., |
| nanocrystals | | | | | | | 2020, 59, 247 |
| Peptidoglycan- | 745 | 1050 | 305 | — | — | F | Angew. Chem., 2020, 132, |
| based | | | | | | | 2650 |
| fluorophores | | | | | | | |
| Organic small | 770 | 1050 | 280 | — | 0.104 | F | Adv. Funct. Mater., 2020, |
| molecule CQ-T ^{e)} | | | | | | | 30, 1906343 |
| Narrow-band- | 800 | 1000 | 200 | — | 0.22 | F | Biomaterials, 2020, 231, |
| gap | | | | | | | 119671 |
| fluorophores ^{f)} | | | | | | | |
| Thiophene- | 750 | 1040 | 290 | — | 0.6 | F | Chem. Mater., 2020, DOI. |
| based | | | | | | | org/10.1021/9b05159 |
| fluorophores | | | | | | | |
| Cell membrane- | 800 | 1060 | 260 | — | 0.71 | F | Chem. Eng. J., 2020, 385, |
| coated rare | | | | | | | 123959 |
| earth | | | | | | | |
| IR820 Dye- | 808 | >1000 | — | — | — | F | Adv. Optical Mater., 2020, |
| Protein | | | | | | | 8, 1901471 |
| Complex | | | | | | | |
| Thiadiazole- | 808 | 1050 | 242 | — | 0.05 | F | Chem. Commun., 2020, |
| based | | | | | | | DOI: 10.1039/C9CC09865H |
| fluorophores | | | | | | | |
| Rhomboidal | 800 | 1100 | 300 | — | 0.03 | F | Proc. Natl. Acad. Sci. USA, |
| Pt(II) | | | | | | | 2019, 116, 1968 |
| metallacycle | | | | | | | |
| Pentamethine | 1015 | 1065 | 50 | — | 0.09 | F | Nat. Commun., 2019, 10, |
| cyanine dyes | | | | | | | 1058 |
| Cu-doped Au | 808 | 1050 | 242 | — | 0.67 | F | Adv. Mater., 2019, 31, |
| nanoclusters | | | | | | | 1901015 |
| Semiconducting | 750 | 1120 | 370 | — | 0.23 | F | Adv. Mater., 2019, 31, |
| polymer brush | | | | | | | 1901187 |
| PBT conjugated | 980 | 1156 | 176 | — | 0.1 | F | Adv. Mater., 2019, 31, |
| polymer ^{g)} | | | | | | | 1902504 |
| Tm³⁺-sensitized | 808 | 1180 | 372 | 70-260 | 0.05-0.2 | F | Angew. Chem. Int. Ed., |
| lanthanide | | | | | | | 2019, 58, 10153 |
| Graphene- | 980 | 1525 | 545 | 290-750 | — | F | Angew. Chem. Int. Ed., |
| Oxide-doped | | | | | | | 2019, 58, 18981 |
| Lanthanide | | | | | | | |
| Rare earth | 808 | 1064 | 256 | — ^{f)} | 0.82 | F | Nano Lett., 2019, 79, 5, |
| doped | | | | | | | 2985 |
| nanoparticles | | | | | | | |
| Fluorophore- | 725 | 1028 | 303 | — | 1.8 | F | Chem. Sci., 2019, 10, 326 |
| peptide | | | | | | | |
| conjugates | | | | | | | |
| Lanthanide- | 808 | 1060 | 252 | 5.8 | 0.009 | F | Nat. Nanotech., 2018, 73, |
| doped | | | | | | | 941 |
| nanocrystals | | | | | | | |
| Organic | 760 | 1050 | 290 | — | 16.5 | F | Nat. Commun., 2018, 9, |
| nanofluorophore ^{h)} | | | | | | | 1171 |
| Rare-earth | 980 | 1550 | 570 | — | 0.27-2.73 | F | Nat. Commun., 2017, 8, |
| nanocrystals | | | | | | | 737 |
| NIR-II organic | 738 | 1055 | 317 | — | 0.2-1.1 | F | Nat. Commun., 2017, 8, |
| molecule ⁱ⁾ | | | | | | | 15269 |
| PbS | — | 1600 | — | — | 2.2-22 | F | Proc. Natl. Acad. Sci. USA, |
| nanocrystals | | | | | | | 2018, 115, 6590 |
| Small molecule | 630 | 810 | 180 | — | 13.9 | F | Adv. Mater., 2018, |
| probe: TQ-BPN ^{j)} | | | | | | | 1706856 |
| Small molecule | 740 | 975 | 235 | — | 0.62 | F | Adv. Mater., 2018, |
| probe: TB1 ^{k)} | | | | | | | 1800766 |
| N/S-doped | 405 | 510 | 105 | <0.008 | 41.2 | F | Angew. Chem. Int. Ed., |
| carbon QDs ^{l)} | | | | | | | 2018, 57, 2377 |
| NIR-II molecule- | 850 | 1120 | 270 | — | 0.1 | F | Adv. Healthcare Mater., |
| protein complex ⁱ⁾ | | | | | | | 2018, 7, 1800589 |
| [symbol] | [symbol] | [symbol] | [symbol] | [symbol] | — | [symbol] | [symbol] |
| [symbol] | | | | | | | [symbol] |
| [symbol] | | | | | | | |
| Cu clusters | 415 | 615 | 200 | 9.7 | 3.4-8.3 | F | ACS Omega, 2018, 3, |
| assembles | | | | | | | 14755 |
| Cu nanoclusters | 385 | 498 | 113 | <0.003 | 6.6 | F | Nanoscale, 2018, 10, 6467 |
| Au nanoclusters | 380 | 630 | 250 | 0.5 | 10 | F | Anal. Chem., 2018, 90, |
| | | | | | | | 7283 |
| Pt nanoclusters | 365 | 500 | 135 | <0.002 | 5.5 | F | Materials, 2018, 11, 191 |
| Cu nanoclusters | 371 | 489 | 118 | about | 6.2 | F | J. Phys. Chem. C, 2018, |
| | | | | 0.001 | | | 722, 13354 |
| Cu clusters | 375 | 450 | 75 | 0.007 | — | F | J. Phys. Chem. C, 2018, |
| emulsion | | | | | | | 722, 5742 |
| N-doped carbon | about | about | — | 0.021 | 20 | F | Nanoscale, 2018, 10, 5342 |
| QDs | 400 | 450 | | | | | |
| Au clusters | 410 | 510 | 100 | 12.3 | 0.4-4.6 | F | ACS Appl. Mater. Interfaces, |
| aggregates | | | | | | | 2018, 10, 19459 |
| [symbol] | [symbol] | [symbol] | [symbol] | [symbol] | [symbol] | [symbol] | [symbol] |
| [symbol] | | | | | | | [symbol] |
| CuInS₂@ZnS | 700 | 1050 | 350 | 0.6 | 6 | F | Chem. Mater., 2017, 29, |
| QDs | | | | | | | 4940 |
| CuInS₂@ZnS | — | 800 | — | 0.51 | 26-38 | F | RSC Adv., 2017, 7, 10675 |
| QDs | | | | | | | |
| Ag₂S | 500 | 1100 | 600 | 0.01 | 13 | F | Anal. Chem., 2017, 89, |
| nanocrystals | | | | | | | 6616 |
| [symbol] | [symbol] | [symbol] | [symbol] | [symbol] | [symbol] | [symbol] | [symbol] |
| [symbol] | | | | | | | [symbol] |
| CdS@ZnS QDs | 404 | 580 | 176 | 0.008 | 12 | F | J. Am. Chem. Soc., 2017, |
| | | | | | | | 139, 8878 |
| Graphitic C₃N₄ | 410 | 525 | 115 | 0.008 | — | F | RSC Adv., 2016, 6, 92839 |
| nanosheets | | | | | | | |
| CuInS₂ QDs | 700 | 980 | 280 | 0.5 | <6.6 | F | J. Phys. Chem. Lett., 2016, |
| | | | | | | | 7, 572 |
| Au in layered | 360 | 560 | 200 | 14.7 | 14.1 | F | Adv. Funct. Mater., 2015, |
| double | | | | | | | 25, 5006 |
| hydroxides | | | | | | | |
| CuInS₂ QDs | 500 | 900 | 400 | <0.5 | 5-10 | F | Chem. Mater., 2015, 27, |
| | | | | | | | 621 |
| Ag₂S QDs | 785 | 1175 | 390 | 0.18 | — | F | J. Phys. Chem. C, 2014, |
| | | | | | | | 118, 4918 |
| Au@Ag | 520 | 667 | 147 | 1.45 | 15 | F | Nanoscale, 2014, 6, 157 |
| clusters | | | | | | | |
| aggregates | | | | | | | |
| Cu clusters | 345 | 640 | 295 | 151 | 16.6 | F | Chem. Commun., 2014, |
| aggregates | | | | | | | 50, 237 |
| Cu clusters | 345 | 600 | 255 | 0.011 | 6.6 | F | Small, 2013, 9, 3873 |
| aggregates | | | | | | | |
| CuInS₂/ZnS | 490 | 530 | 40 | 0.69 | 24 | F | ACS Appl. Mater. Interfaces, |
| QDs | | | | | | | 2013, 5, 8210 |
| Au clusters | 330 | 565 | 235 | 2.93 | 15 | F | J. Am. Chem. Soc., 2012, |
| aggregates | | | | | | | 134, 16662 |
| Pt(II) | 365 | 560 | 195 | 0.16, 0.43 | — | F | J. Mater. Chem., 2012, 22, |
| complexes | | | | | | | 22167 |

^{a)} From steady-state photoluminescence spectra upon emission at λₑₘ.
^{b)} From steady-state photoluminescence spectra upon excitation at the characteristic absorption band or at 808 nm.
^{c)} The character “P” represented that the probes emit phosphorescence.
^{d)} The character “F” represented that the probes emit fluorescence.
^{e)} CQ-T: Donor-acceptor-donor scaffold with 3,4-ethylenedioxythiophene and dialkylfluorene.
^{f)} The fluorophores was synthesized by indacenobis(dithieno[3,2-b:2′,3′-d]pyrrol) core with 2-butyl-1-octyl side chains and chlorinated (dicyanomethylidene)-indan-1-one.
^{g)} PBT: poly(benzodithiophene-alt-thiadiazolobenzotriazole).
^{h)} Nanofluorophore was composed of benzobisthiadiazole as an acceptor, 3,4-ethylenedioxy thiophene as a donor, and dialkyl fluorene as a shield unit.
ⁱ⁾ NIR-II molecule was composed of O-benzo-triazole-N,N,N′,N′-tetramethyl-uronium-hexafluorophosphate and N,N-diisopropylethylamine.
^{j)} TQ-BPN: N,N-diphenylnaphthalen-1-amine (BPN), thiadiazolo[3,4-g]quinoxaline (TQ).
^{k)} TB1: N-phenyl-N-(4-(1,2,2-triphenylvinyl)phenyl)aniline derivate.
^{l)} “QDs” represented “quantum dots”.

Taking a closer look at Table 3, it was found that despite substantial achievements of the NI R-II-related theory and applications, examples of probes that were capable of emitting phosphorescence in a NIR-II window were still in their infancy, not to mention tumor hallmarks such as irregular pH or redox activatable NIR-II phosphorescent probes.

### References for Example 2

1. Xu, J., et al. Large-scale synthesis of long crystalline Cu₂₋ₓSe nanowire bundles by water-evaporation-induced self assembly and their application in gas sensing. Adv. Funct.
Mater. 19, 1759-1766 (2009).
2. Lu, X. T., Zhuang, Z. B., Peng, Q. & Li, Y. D. Controlled synthesis of wurtzite CuInS₂ nanocrystals and their side-by-side nanorod assemblies. Crystengcomm 13, 4039-4045 (2011).
3. Wang, D., et al. How and why nanoparticle's curvature regulates the apparent pKₐ of the coating ligands. J. Am. Chem. Soc. 133, 2192-2197 (2011).
4. De Trizio, L. & Manna, L. Forging colloidal nanostructures via cation exchange reactions. Chem. Rev. 116, 10852-10887 (2016).
5. Liu, H., et al. Morphological growth and phase formation of CuInSe₂ nanocrystals by an ambient pressure polylol-based solution synthesis. CrystEngComm 14, 8186-8192 (2012).
6. Lox, J. F. L., et al. Near-infrared Cu—In—Se-based colloidal nanocrystals via cation exchange. Chem. Mater. 30, 2607-2617 (2018).
7. Chen, Y.-S., Zhao, Y., Yoon, S. J., Gambhir, S. S. & Emelianov, S. Miniature gold nanorods for photoacoustic molecular imaging in the second near-infrared optical window. Nat. Nanotechnol. 14, 465-472 (2019).
8. Huang, P., Jiang, Q., Yu, P., Yang, L. & Mao, L. Alkaline post-treatment of Cd(II)-glutathione coordination polymers: Toward green synthesis of water-soluble and cytocompatible CdS quantum dots with tunable optical properties. ACS Appl. Mater. Interfaces 5, 5239-5246 (2013).
9. Jana, A., et al. Investigating the control by quantum confinement and surface ligand coating of photocatalytic efficiency in chalcopyrite copper indium diselenide nanocrystals. Chem. Mater. 28, 1107-1120 (2016).
10. Robinson, R. D., et al. Spontaneous superlattice formation in nanorods through partial cation exchange. Science 317, 355-358 (2007).
11. Leach, A. D. P. & Macdonald, J. E. Optoelectronic properties of CuInS₂ nanocrystals and their origin. J. Phys. Chem. Led. 7, 572-583 (2016).
12. Jin, R. C., Zeng, C. J., Zhou, M. & Chen, Y. X. Atomically precise colloidal metal nanoclusters and nanoparticles: Fundamentals and opportunities. Chem. Rev. 116, 10346-10413 (2016).
13. Zhang, H., et al. Clusterization-triggered emission: Uncommon luminescence from common materials. Mater. Today 32, 275-292 (2019).
14. Charron, G., et al. On the use of pH titration to quantitatively characterize colloidal nanoparticles. Langmuir 28, 15141-15149 (2012).
15. Luo, Z. T., et al. From aggregation-induced emission of Au(I)-thiolate complexes to ultrabright Au(0)©Au(I)-thiolate core-shell nanoclusters. J. Am. Chem. Soc. 134, 16662-16670 (2012).
16. Ni, W. H., Mosquera, R. A., Perez-Juste, J. & Liz-Marzan, L. M. Evidence for hydrogen-bonding-directed assembly of gold nanorods in aqueous solution. J. Phys. Chem. Lett. 1, 1181-1185 (2010).
17. Ji, C., et al. Temperature-Dependent Photoluminescence of Ag₂Se Quantum Dots. J. Phys. Chem. C 119, 13841-13846 (2015).
18. Jakob, M., et al. Reappraising the Luminescence Lifetime Distributions in Silicon Nanocrystals. Nanoscale Res. Led. 13, 383 (2018).
19. Kim, Y. K., Ahn, S. H., Chung, K., Cho, Y. S. & Choi, C. J. The photoluminescence of CuInS₂ nanocrystals: Effect of non-stoichiometry and surface modification. J. Mater. Chem. 22, 1516-1520 (2012).
20. Zhao, W. J., et al. Boosting the efficiency of organic persistent room-temperature phosphorescence by intramolecular triplet-triplet energy transfer. Nat. Commun. 10, 1595 (2019).
21. Zhou, P., et al. Temperature-dependent photoluminescence properties of Mn:ZnCuInS nanocrystals. Opt. Mater. Express 5, 2069-2080 (2015).
22. Seo, J., et al. Time-resolved and temperature-dependent photoluminescence of ternary and quaternary nanocrystals of CuInS₂ with ZnS capping and cation exchange. J. Appl. Phys. 114, 094310 (2013).
23. Wang, J., Zhang, M., Zhang, Q., Ding, W. & Su, Q. The photoluminescence and thermoluminescence properties of novel green long-lasting phosphorescence materials Ca8Mg(SiO4)(4)Cl-2: Eu2+, Nd3+. Appl Phys B-Lasers O 87, 249-254 (2007).
24. Tian, B. N., et al. Excitation pathway and temperature dependent luminescence in color tunable Ba₅Gd₈Zn₄O₂₁:Eu³⁺ phosphors. J. Mater. Chem. C 1, 2338-2344 (2013).
25. Green, T. D., et al. Temperature-dependent photoluminescence of structurally-precise quantum-confined Au₂₅(SC₈H₉)₁₈ and Au₃₈(SC₁₂H₂₅)₂₄ Metal Nanoparticles. J. Phys. Chem. A 118, 10611-10621 (2014).
26. Ding, Q., et al. Temperature dependent photoluminescence of composition tunable ZnxAglnSe quantum dots and temperature sensor application. Opt. Express 25, 19065-19076 (2017).
27. Li, C. & Wang, Q. Challenges and opportunities for intravital near-infrared fluorescence imaging technology in the second transparency window. ACS Nano 12, 9654-9659 (2018).
28. Liu, Y., Yao, D. & Zhang, H. Self-assembly driven aggregation-induced emission of copper nanoclusters: A novel technology for lighting. ACS Appl. Mater. Interfaces 10, 12071-12080 (2018).
29. Berends, A. C., Mangnus, M. J. J., Xia, C. H., Rabouw, F. T. & Donega, C. D. Optoelectronic properties of ternary I-III-VI₂ semiconductor nanocrystals: Bright prospects with elusive origins. J. Phys. Chem. Lett. 10, 1600-1616 (2019).
30. Zhang, M., et al. Bright quantum dots emitting at −1,600 nm in the NIR-Ilb window for deep tissue fluorescence imaging. P. Natl. Acad. Sci. USA 115, 6590-6595 (2018).
31. Wang, Y. G., et al. A nanoparticle-based strategy for the imaging of a broad range of tumours by nonlinear amplification of microenvironment signals. Nat. Mater. 13, 204-212 (2014).
32. Faubert, B., et al. Lactate metabolism in human lung tumors. Cell 171, 358-371 (2017).
33. Schwartz, L., Supuran, C. T. & Alfarouk, K. O. The Warburg effect and the hallmarks of cancer. Anti-Cancer Agents Med. Chem. 17, 164-170 (2017).
34. Fan, Y., et al. Lifetime-engineered NIR-II nanoparticles unlock multiplexed in vivo imaging. Nat. Nanotechnol. 13, 941-946 (2018).
35. van der Stam, W., et al. Luminescent CuInS₂ quantum dots by partial cation exchange in Cu₂₋ₓS nanocrystals. Chem. Mater. 27, 621-628 (2015).
36. Antaris, A. L., et al. A high quantum yield molecule-protein complex fluorophore for near-infrared II imaging. Nat. Commun. 8, 15269 (2017).

1. An imaging probe comprising:
a CuInX₂ nanotube, wherein X is a chalcogen selected from S, Se, and Te, the CuInX₂ nanotube comprising an outer diameter and an inner diameter defining a hollow center, wherein the CuInX₂ nanotube emits a weak florescence at a pH of about 7.0 or higher and is configured to emit detectable NIR-II phosphorescence upon aggregation with a plurality of other CuInSe₂ nanotubes at a pH of about 6.8 or lower.

2. The imaging probe of claim 1, wherein the CuInX₂ nanotube comprises a capping/ligand moiety selected from glutathione (GSH) and cystine.

3. The imaging probe of claim 1, wherein the nanotube has an outer diameter of about 5 nm to about 20 nm and an inner diameter of about 2 nm to about 10 nm.

4. The imaging probe of claim 1, wherein the nanotube has a thickness, between the inner diameter and outer diameter, of about 2 nm to about 8 nm.

5. The imaging probe of claim 1, wherein the nanotube comprises an outer portion near the outer diameter and an inner portion near the hollow center, wherein the outer portion has a composition of predominately CuInSe₂ and the inner portion has a composition having a greater amount of In₂Se₃ nanoparticles than the outer portion.

6. The imaging probe of claim 1, wherein X is Selenium and the CuInX₂ nanotube is a CuInSe₂ nanotube.

7. The imaging probe of claim 6, wherein the CuInSe₂ nanotube emits phosphorescence at about 1130 nm at a pH of about 6.5 to about 6.8.

8. The imaging probe of claim 6, wherein the CuInSe₂ nanotube experiences a Stokes shift in emission intensity of about 424 nm over a change in pH of about 0.4.

9. A pharmaceutically acceptable imaging composition comprising a plurality of imaging probes of claim 1 and a pharmaceutically acceptable carrier.

10. A method of generating an image of a tissue in an animal or human subject, the method comprising
administering to an animal or human subject a pharmaceutically acceptable composition comprising a plurality of CuInX₂ nanotubes, wherein X is a chalcogen selected from S, Se, and Te, wherein the CuInX₂ nanotubes emit weak fluorescence at an environmental pH of about 7.0 or higher and wherein the CuInX₂ nanotubes form nanoaggregates and emit NIR-II phosphorescence in a second near-infrared range of about 1000-1700 nm at an environmental pH of about 6.8 or lower; and
obtaining an image of the location of nanoaggregates of the CuInX₂ nanotubes in a tissue of the animal or human subject by detecting and imaging the phosphorescence.

11. The method of claim 10, wherein X is Selenium and the CuInX₂ nanotube is a CuInSe₂ nanotube.

12. The method of claim 11, wherein the CuInSe₂ nanotubes aggregate and emit phosphorescence at about 1130 nm at a pH of about 6.5 to about 6.8.

13. The method of claim 11 wherein the CuInSe₂ nanotubes produce a Stokes shift in emission intensity of about 424 nm over a change in pH of about 0.4.

14. The method of claim 11, wherein the image of the location of nanoaggregates of the CuInSe₂ nanotubes is obtained with an imaging system configured to detect phosphorescence in a second near infrared range of about 1000-1700 nm.

15. The method of claim 11, further comprising imaging cancer in the animal or human subject by obtaining the image of the location of nanoaggregates of the CuInSe₂ nanotubes in the tissue of the animal or human subject by detecting and imaging the phosphorescence, wherein the location of nanoaggregates of the CuInSe₂ nanotubes indicates the location of cancer in the subject.

16. The method of claim 15, wherein a tumor-to-normal-tissue (T/NT) signal ratio for CuInSe₂ nanotubes is above about 5.

17. The method of claim 15, wherein a tumor-to-normal-tissue (T/NT) signal ratio for CuInSe₂ nanotubes is from about 180 to about 200 at about 24 hours post administration.

18. The method of claim 15, wherein a tumor-to-liver (T/L) phosphorescent signal ratio for CuInSe₂ nanotubes is about 170 to about 150.

19. A system for generating an image of a tissue in an animal or human subject, the system comprising:
a pharmaceutically acceptable imaging composition comprising a plurality of imaging probes of claim 1 and a pharmaceutically acceptable carrier; and
an imaging system configured to detect phosphorescence in a second near infrared range of about 1000-1700 nm.

20. A method of making a CuInSe₂ nanotube, the method comprising the steps of:
a) synthesizing Cu₂₋ₓSe solid nanorods by water-evaporation-induced self-assembly; and
b) reduction of the Cu₂₋ₓSe nanorods with NaBH₄ to form hollow CuInSe₂ nanotubes.
